(19) United States
(12) Patent Application Publication
(10) Pub. No.: US 2010/0184758 A1
(43) Pub. Date: Jul. 22, 2010

(54) BETA CARBOLINE DERIVATIVES AS ANTIDIABETIC COMPOUNDS

(76) Inventors: Peter H. Dobbelaar, Morris Plains, NJ (US); Wu Du, Monroe Township, NJ (US); Liangqin Guo, Edison, NJ (US); William K. Hagmann, Westfield, NJ (US); Shuwen He, Edison, NJ (US); Tianying Jian, Westfield, NJ (US); Jian Liu, Edison, NJ (US); Ravi P. Nargund, East Brunswick, NJ (US); Alexander Pasternak, Princeton, NJ (US); Shrenik K. Shah, Metuchen, NJ (US); Quang T. Truong, Morganville, NJ (US); Zhixiong Ye, Princeton, NJ (US); James Deliureficio, Millington, NJ (US); Raman Bakshi, Edison, NJ (US)

Correspondence Address: MERCK, P O BOX 2000, RAHWAY, NJ, 07065-0907, US

(21) Appl. No.: 12/668,822

(22) PCT Filed: Jul. 15, 2008
(86) PCT No.: PCT/US08/08611

§ 371 Date: Jan. 12, 2010

Related U.S. Application Data
(60) Provisional application No. 60/961,194, filed on Jul. 19, 2007.

(51) Int. Cl. A61K 31/5377; C07D 471/14; A61K 31/437; C07D 413/14; C07D 403/14; A61K 31/501; A61K 31/4995; A61K 31/506; A61P 3/10; A61P 3/04; A61P 3/00; A61P 3/06; A61P 9/12

(52) U.S. Cl. 514/232.8; 546/84; 514/292; 544/126; 544/238; 514/252.04; 544/361; 514/253.03; 544/333; 514/256; 544/315; 514/274

(57) ABSTRACT

Beta-carboline derivatives of structural formula I are selective antagonists of the somatostatin subtype receptor 3 (SSTR3) and are useful for the treatment of Type 2 diabetes mellitus and of conditions that are often associated with this disease, including hyperglycemia, insulin resistance, obesity, lipid disorders, and hypertension. The compounds are also useful for the treatment of depression and anxiety.

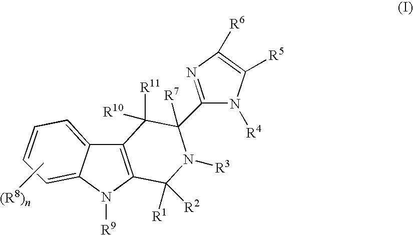

### FIELD OF THE INVENTION

The instant invention is concerned with substituted beta-carboline derivatives, which are selective antagonists of the somatostatin subtype receptor 3 (SSTR3) which are useful for the treatment of Type 2 diabetes mellitus and of conditions that are often associated with this disease, including hyperglycemia, insulin resistance, obesity, lipid disorders, and hypertension.

The compounds are also useful for the treatment of depression and anxiety.

### BACKGROUND OF THE INVENTION

Diabetes is a disease derived from multiple causative factors and characterized by elevated levels of plasma glucose (hyperglycemia) in the fasting state or after administration of glucose during an oral glucose tolerance test. There are two generally recognized forms of diabetes. In type 1 diabetes, or insulin-dependent diabetes mellitus (IDDM), patients produce little or no insulin, the hormone which regulates glucose utilization. In Type 2 diabetes, or noninsulin-dependent diabetes mellitus (NIDDM), insulin is still produced by islet cells in the pancreas. Patients having Type 2 diabetes have a resistance to the effects of insulin in stimulating glucose and lipid metabolism in the main insulin-sensitive tissues, including muscle, liver and adipose tissues. These patients often have normal levels of insulin, and may have hyperinsulinemia (elevated plasma insulin levels), as they compensate for the reduced effectiveness of insulin by secreting increased amounts of insulin (Polonsky, Int. J. Obes. Relat. Metab. Disord. 24 Suppl 2:S29-31, 2000). The beta cells within the pancreatic islets initially compensate for insulin resistance by increasing insulin output. Insulin resistance is not primarily caused by a diminished number of insulin receptors but rather by a post-insulin receptor binding defect that is not yet completely understood. This lack of responsiveness to insulin results in insufficient insulin-mediated activation of uptake, oxidation and storage of glucose in muscle, and inadequate insulin-mediated repression of lipolysis in adipose tissue and of glucose production and secretion in the liver. Eventually, a patient may be become diabetic due to the inability to properly compensate for insulin resistance. In humans, the onset of Type 2 diabetes due to insufficient increases (or actual declines) in beta cell mass is apparently due to increased beta cell apoptosis relative to non-diabetic insulin resistant individuals (Butler et al., Diabetes 52:102-110, 2003).

Persistent or uncontrolled hyperglycemia that occurs with diabetes is associated with increased and premature morbidity and mortality. Often abnormal glucose homeostasis is associated both directly and indirectly with obesity, hypertension, and alterations of the lipid, lipoprotein and apolipoprotein metabolism, as well as other metabolic and hemodynamic disease. Patients with Type 2 diabetes mellitus have a significantly increased risk of macrovascular and microvascular complications, including atherosclerosis, coronary heart disease, stroke, peripheral vascular disease, hypertension, nephropathy, neuropathy, and retinopathy. Therefore, effective therapeutic control of glucose homeostasis, lipid metabolism, obesity, and hypertension are critically important in the clinical management and treatment of diabetes mellitus.

Patients who have insulin resistance often exhibit several symptoms that together are referred to as syndrome X or Metabolic Syndrome. According to one widely used definition, a patient having Metabolic Syndrome is characterized as having three or more symptoms selected from the following group of five symptoms: (1) abdominal obesity, (2) hypertriglyceridemia, (3) low levels of high-density lipoprotein cholesterol (HDL), (4) high blood pressure, and (5) elevated fasting glucose, which may be in the range characteristic of Type 2 diabetes if the patient is also diabetic. Each of these symptoms is defined clinically in the Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with Metabolic Syndrome, whether they have or develop overt diabetes mellitus, have an increased risk of developing the macrovascular and microvascular complications that occur with Type 2 diabetes, such as atherosclerosis and coronary heart disease.

There are several available treatments for Type 2 diabetes, each of which has its own limitations and potential risks. Physical exercise and a reduction in dietary intake of calories often dramatically improves the diabetic condition and are the usual recommended first-line treatment of Type 2 diabetes and of pre-diabetic conditions associated with insulin resistance. Compliance with this treatment is generally very poor because of well-entrenched sedentary lifestyles and excess food consumption, especially of foods containing high amounts of fat and carbohydrates. Pharmacologic treatments have largely focused on three areas of pathophysiology: (1) hepatic glucose production (biguanides), (2) insulin resistance (PPAR agonists), (3) insulin secretion (sulfonylureas); (4) incretin hormone mimetics (GLP-1 derivatives and analogs, such as exenatide and luraglitide); and (5) inhibitors of incretin hormone degradation (DPP-4 inhibitors).

The biguanides belong to a class of drugs that are widely used to treat Type 2 diabetes. Phenformin and metformin are the two best known biguanides and do cause some correction of hyperglycemia. The biguanides act primarily by inhibiting hepatic glucose production, and they also are believed to modestly improve insulin sensitivity. The biguanides can be used as monotherapy or in combination with other anti-diabetic drugs, such as insulin or insulin secretagogues, without increasing the risk of hypoglycemia. However, phenformin and metformin can induce lactic acidosis, nausea/vomiting, and diarrhea. Metformin has a lower risk of side effects than phenformin and is widely prescribed for the treatment of Type 2 diabetes.

The glitazones (e.g., 5-benzylthiazolidine-2,4-diones) are a class of compounds that can ameliorate hyperglycemia and other symptoms of Type 2 diabetes. The glitazones that are currently marketed (rosiglitazone and pioglitazone) are agonists of the peroxisome proliferator activated receptor (PPAR) gamma subtype. The PPAR-gamma agonists substantially increase insulin sensitivity in muscle, liver and adipose tissue in several animal models of Type 2 diabetes, resulting in partial or complete correction of elevated plasma glucose levels without the occurrence of hypoglycemia. PPAR-gamma agonism is believed to be responsible for the improved insulin sensititization that is observed in human patients who are treated with the glitazones. New PPAR agonists are currently being developed. Many of the newer PPAR compounds are agonists of one or more of the PPAR alpha, gamma and delta subtypes. The currently marketed PPAR gamma agonists are modestly effective in reducing plasma glucose and hemoglobinA1C. The currently marketed compounds do not greatly improve lipid metabolism and may actually have a negative effect on the lipid profile. Thus, the PPAR compounds represent an important advance in diabetic therapy.

Another widely used drug treatment involves the administration of insulin secretagogues, such as the sulfonylureas (e.g., tolbutamide, glipizide, and glimepiride). These drugs increase the plasma level of insulin by stimulating the pancreatic β-cells to secrete more insulin. Insulin secretion in the pancreatic β-cell is under strict regulation by glucose and an array of metabolic, neural and hormonal signals. Glucose stimulates insulin production and secretion through its metabolism to generate ATP and other signaling molecules, whereas other extracellular signals act as potentiators or inhibitors of insulin secretion through GPCR's present on the plasma membrane. Sulfonylureas and related insulin secretagogues act by blocking the ATP-dependent K⁺ channel in β-cells, which causes depolarization of the cell and the opening of the voltage-dependent Ca2+ channels with stimulation of insulin release. This mechanism is non-glucose dependent, and hence insulin secretion can occur regardless of the ambient glucose levels. This can cause insulin secretion even if the glucose level is low, resulting in hypoglycemia, which can be fatal in severe cases. The administration of insulin secretagogues must therefore be carefully controlled. The insulin secretagogues are often used as a first-line drug treatment for Type 2 diabetes.

Dipeptidyl peptidase-IV (DPP-4) inhibitors (e.g., sitagliptin, vildagliptin, saxagliptin, and alogliptin) provide a new route to increase insulin secretion in response to food consumption. Glucagon-like peptide-1 (GLP-1) levels increase in response to the increases in glucose present after eating and glucagon stimulates the production of insulin. The serine proteinase enzyme DPP-4 which is present on many cell surfaces degrades GLP-1. DPP-4 inhibitors reduce degradation of GLP-1, thus potentiating its action and allowing for greater insulin production in response to increases in glucose through eating.

There has been a renewed focus on pancreatic islet-based insulin secretion that is controlled by glucose-dependent insulin secretion. This approach has the potential for stabilization and restoration of β-cell function. In this regard, the present application claims compounds that are antagonists of the somatostatin subtype receptor 3 (SSTR3) as a means to increase insulin secretion in response to rises in glucose resulting from eating a meal. These compounds may also be used as ligands for imaging (e.g., PET, SPECT) for assessment of beta cell mass and islet function. A decrease in β-cell mass can be determined with respect to a particular patient over the course of time.

### SUMMARY OF THE INVENTION

The present invention is directed to compounds of structural formula I, and pharmaceutically acceptable salts thereof:

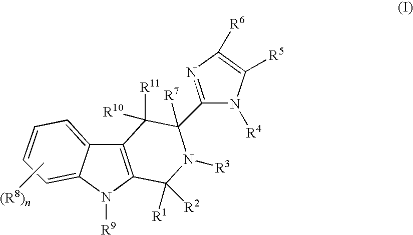

These bicyclic beta-carboline derivatives are effective as antagonists of SSTR3. They are therefore useful for the treatment, control or prevention of disorders responsive to antagonism of SSTR3, such as Type 2 diabetes, insulin resistance, lipid disorders, obesity, atherosclerosis, Metabolic Syndrome, depression, and anxiety.

The present invention also relates to pharmaceutical compositions comprising the compounds of the present invention and a pharmaceutically acceptable carrier.

The present invention also relates to methods for the treatment, control, or prevention of disorders, diseases, or conditions responsive to antagonism of SSTR3 in a subject in need thereof by administering the compounds and pharmaceutical compositions of the present invention.

The present invention also relates to methods for the treatment, control, or prevention of Type 2 diabetes, hyperglycemia, insulin resistance, obesity, lipid disorders, atherosclerosis, and Metabolic Syndrome by administering the compounds and pharmaceutical compositions of the present invention.

The present invention also relates to methods for the treatment, control, or prevention of depression and anxiety by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating Metabolic Syndrome by administering the compounds of the present invention in combination with a therapeutically effective amount of another agent known to be useful to treat the condition.

The present invention also relates to methods for the treatment, control, or prevention of depression and anxiety by administering the compounds of the present invention in combination with a therapeutically effective amount of another agent known to be useful to treat the condition.

Another aspect of the present invention relates to methods for the treatment of Type 2 diabetes, hyperglycemia, insulin resistance, and obesity with a therapeutically effective amount of an SSTR3 antagonist in combination with a therapeutically effective amount of a dipeptidyl peptidase-IV (DPP-4) inhibitor.

Another aspect of the present invention relates to the use of an SSTR3 antagonist in combination with a DPP-4 inhibitor for the manufacture of a medicament for treating Type 2 diabetes, hyperglycemia, insulin resistance, and obesity.

### DETAILED DESCRIPTION OF THE INVENTION

The present invention is concerned with beta-carboline derivatives useful as antagonists of SSTR3. Compounds of the present invention are described by structural formula I

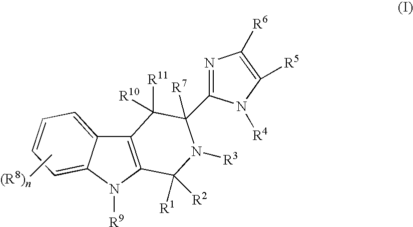

and pharmaceutically acceptable salts thereof, wherein:
n is an integer from 1 to 4;
R¹ is selected from the group consisting of:

(1) —C(O)OR^{e},

(2) —C(O)NR^{c}R^{d},

(3) cycloheteroalkyl,

(4) cycloheteroalkyl-C₁₋₁₀ alkyl-,

(5) heteroaryl, and

(6) heteroaryl-C₁₋₁₀ alkyl-;

wherein alkyl and cycloheteroalkyl are optionally substituted with one to three substituents independently selected from R^{a}; and heteroaryl is optionally substituted with one to three substituents independently selected from R^{b};
with the proviso that heteroaryl is not pyridinyl, pyrrolyl, thienyl, 1,3-benzodioxolyl, or furanyl;
R² is selected from the group consisting of

hydrogen,

C₁₋₁₀ alkyl,

C₂₋₁₀ alkenyl,

C₂₋₁₀ alkynyl,

C₃₋₁₀ cycloalkyl,

C₃₋₁₀ cycloalkyl-C₁₋₁₀ alkyl-,

C₁₋₆ alkyl-X—C₁₋₆ alkyl-,

aryl-C₁₋₄ alkyl-X—C₁₋₄ alkyl-,

heteroaryl-C₁₋₄ alkyl-X—C₁₋₄ alkyl-,

C₃₋₁₀ cycloalkyl-X—C₁₋₆ alkyl-,

aryl,

cycloheteroalkyl, and

heteroaryl;

wherein X is selected from the group consisting of O, S, S(O), S(O)₂, and NR⁴ and wherein alkyl, alkenyl, alkynyl, cycloalkyl, and cycloheteroalkyl are optionally substituted with one to three substituents independently selected from R^{a}; and aryl and heteroaryl are optionally substituted with one to three substituents independently selected from R^{b};
R³ is selected from the group consisting of

hydrogen,

C₁₋₁₀ alkyl,

C₃₋₁₀ cycloalkyl,

cycloheteroalkyl,

cycloheteroalkyl-C₁₋₆ alkyl-, and

heteroaryl-C₁₋₆ alkyl-;

wherein alkyl, cycloalkyl, and cycloheteroalkyl are optionally substituted with one to three substituents independently selected from R^{a}; and heteroaryl is optionally substituted with one to three substituents independently selected from R^{b};
R⁴ is hydrogen or C₁₋₈ alkyl, optionally substituted with one to five fluorines;
R⁵ and R⁶ are each independently selected from the group consisting of

hydrogen,

C₁₋₁₀ alkyl,

C₂₋₁₀ alkenyl,

C₂₋₁₀ alkynyl,

C₃₋₁₀ cycloalkyl,

cycloheteroalkyl,

aryl, and

heteroaryl;

wherein alkyl, cycloalkyl, and cycloheteroalkyl are optionally substituted with one to three substituents independently selected from R^{a}, and aryl and heteroaryl are optionally substituted with one to three substituents independently selected from R^{b};
R⁷ is selected from the group consisting of:

hydrogen,

C₁₋₁₀ alkyl, optionally substituted with one to five fluorines,

C₂₋₁₀ alkenyl,

C₃₋₁₀ cycloalkyl, and

C₁₋₄ alkyl-O—C₁₋₄ alkyl-;

each R⁸ is independently selected from the group consisting of:

(1) hydrogen,

(2) —OR^{e},

(3) —NR^{c}S(O)ₘR^{e},

(4) halogen,

(5) —S(O)ₘR^{e},

(6) —S(O)ₘNR^{c}R^{d},

(7) —NR^{c}R^{d},

(8) —C(O)R^{e},

(9) —OC(O)R^{e},

(10) —CO₂R^{e},

(11) —CN,

(12) —C(O)NR^{c}R^{d},

(13) —NR^{c}C(O)R^{e},

(14) —NR^{c}C(O)OR^{e},

(15) —NR^{c}C(O)NR^{c}R^{d};

(16) —OCF₃,

(17) —OCHF₂,

(18) cycloheteroalkyl,

(19) C₁₋₁₀ alkyl, optionally substituted with one to five fluorines,

(20) C₃₋₆ cycloalkyl,

(21) aryl, and

(22) heteroaryl;

wherein aryl and heteroaryl are optionally substituted with one to three substituents independently selected from R^{b};
R⁹ is selected from the group consisting of

hydrogen,

C₁₋₁₀ alkyl,

C₂₋₁₀ alkenyl, and

C₃₋₁₀ cycloalkyl;

wherein alkyl, alkenyl, and cycloalkyl are optionally substituted with one to three substituents independently selected from R^{a};
R¹⁰ and R¹¹ are each independently hydrogen or C₁₋₄ alkyl, optionally substituted with one to five fluorines;
each R^{a} is independently selected from the group consisting of:

(1) —OR^{e},

(2) —NR^{c}S(O)ₘR^{e},

(3) halogen,

(4) —S(O)ₘR^{e},

(5) —S(O)ₘNR^{c}R^{d},

(6) —NR^{c}R^{d},

(7) —C(O)R^{e};

(8) —OC(O)R^{e};

(9) oxo,

(10) —CO₂R^{e},

(11) —CN,

(12) —C(O)NR^{c}R^{d};

(13) —NR^{c}C(O)R^{e},

(14) —NR^{c}C(O)OR^{e},

(15) —NR^{c}C(O)NR^{c}R^{d},

(16) —CF₃,

(17) —OCF₃,

(18) —OCHF₂ and

(19) cycloheteroalkyl;

(20) C₃₋₆ cycloalkyl-C₁₋₆ alkyl; and

(21) C₁₋₆ alkyl-X—C₁₋₆ alkyl-;

wherein X is selected from the group consisting of O, S, S(O), S(O)₂, and NR⁴;
each R^{b} is independently selected from the group consisting of:

(1) R^{a},

(2) C₁₋₁₀ alkyl, and

(3) C₃₋₆ cycloalkyl;

wherein alkyl and cycloalkyl are optionally substituted with one to three hydroxyls and one to six fluorines;
R^{c} and R^{d} are each independently selected from the group consisting of:

(1) hydrogen,

(2) C₁₋₁₀

(3) C₂₋₁₀ alkenyl,

(4) C₃₋₆ cycloalkyl,

(5) C₃₋₆ cycloalkyl-C₁₋₁₀ alkyl-,

(6) cycloheteroalkyl,

(7) cycloheteroalkyl-C₁₋₁₀ alkyl-,

(8) aryl,

(9) heteroaryl,

(10) aryl-C₁₋₁₀ alkyl-, and

(11) heteroaryl-C₁₋₁₀ alkyl-; or

R^{c} and R^{d} together with the atom(s) to which they are attached form a heterocyclic ring of 4 to 7 members containing 0-2 additional heteroatoms independently selected from oxygen, sulfur and N—R^{g};
and, when R^{c} and R^{d} are other than hydrogen, each R^{c} and R^{d} is optionally substituted with one to three substituents independently selected from R^{h};
each R^{e} is independently selected from the group consisting of:

(1) hydrogen,

(2) C₁₋₁₀ alkyl,

(3) C₂₋₁₀ alkenyl,

(4) C₃₋₆ cycloalkyl,

(5) C₃₋₆ cycloalkyl-C₁₋₁₀ alkyl-,

(6) cycloheteroalkyl,

(7) cycloheteroalkyl-C₁₋₁₀ alkyl-,

(8) aryl,

(9) heteroaryl,

(10) aryl-C₁₋₁₀ alkyl-, and

(11) heteroaryl-C₁₋₁₀ alkyl-;

wherein, when R^{e} is not hydrogen, each R^{e} is optionally substituted with one to three substituents selected from R^{h};
each R^{g} is independently —C(O)R^{e} or C₁₋₁₀ alkyl, optionally substituted with one to five fluorines;
each R^{h} is independently selected from the group consisting of:

(1) halogen,

(2) C₁₋₁₀ alkyl,

(3) —O—C₁₋₄ alkyl,

(4) —S(O)ₘ—C₁₋₄ alkyl,

(5) —CN,

(6) —CF₃,

(7) —OCHF₂, and

(8) —OCF₃; and

each m is independently 0, 1 or 2.

The invention has numerous embodiments, which are summarized below. The invention includes compounds of Formula I. The invention also includes pharmaceutically acceptable salts of the compounds and pharmaceutical compositions comprising the compounds and a pharmaceutically acceptable carrier. The compounds are useful for the treatment of Type 2 diabetes, hyperglycemia, obesity; and lipid disorders that are associated with Type 2 diabetes.

In one embodiment of the compounds of the present invention, R³, R⁴, R⁵, R⁹, R¹⁰, and R¹¹ are each hydrogen. In a class of this embodiment, R⁷ is hydrogen or methyl.

In a second embodiment of the compounds of the present invention, R⁴ and R⁵ are hydrogen, and R⁶ is phenyl or heteroaryl each of which is optionally substituted with one to three substituents independently selected from R^{b}. In a class of this embodiment, heteroaryl is pyridinyl optionally substituted with one to two substituents independently selected from R^{b}. In another class of this embodiment, R⁶ is phenyl or pyridin-2-yl optionally substituted with one to two substituents independently selected from the group consisting of halogen, methyl, and methoxy. In a subclass of this class, R⁶ is phenyl, 4-fluorophenyl, pyridin-2-yl, or 5-fluoro-pyridin-2-yl.

In a third embodiment of the compounds of the present invention, n is 1. In a class of this third embodiment R⁸ is hydrogen, halogen, or cyano. In a subclass of this class, R⁸ is hydrogen, chloro, or fluoro. In a subclass of this subclass, R⁸ is hydrogen.

In a fourth embodiment of the compounds of the present invention, R² is selected from the group consisting of:

hydrogen,

heteroaryl, optionally substituted with one to three substituents independently selected from R^{b},

C₁₋₃ alkyl-O—C₁₋₃ alkyl-, and

C₁₋₆ alkyl, wherein alkyl is optionally substituted with one to two substituents independently selected from R^{a}.

In a fifth embodiment of the compounds of the present invention, R¹ is cycloheteroalkyl or heteroaryl wherein cycloheteroalkyl is optionally substituted with one to three substituents independently selected from R^{a}, and heteroaryl is optionally substituted with one to three substituents independently selected from R^{b}. In a class of this fifth embodiment, R¹ is heteroaryl optionally substituted with one to two substituents independently selected from R^{b}. In a subclass of this class, R¹ is heteroaryl selected from the group consisting of 1,2,4-oxadiazol-3-yl, 1,3,4-oxadiazol-2-yl, 1,2,4-thiadiazol-3-yl, pyrazol-3-yl, pyrazol-4-yl, 1,2,3-triazol-4-yl, 1,2,4-triazol-3-yl, 1,3-thiazol-4-yl, 1,3-thiazol-5-yl, and 1,3-oxazol-4-yl, each of which is optionally substituted with C₁₋₄ alkyl wherein alkyl is optionally substituted with one to three fluorines.

In a sixth embodiment of the compounds of the present invention, R¹ is heteroaryl optionally substituted with one to three substituents independently selected from R^{b}, and R² is selected from the group consisting of:

hydrogen,

heteroaryl, optionally substituted with one to three substituents independently selected from R^{b},

C₁₋₃ alkyl-O—C₁₋₃ alkyl-, and

C₁₋₆ alkyl, wherein alkyl is optionally substituted with one to two substituents independently selected from R^{a}.

In a class of this sixth embodiment, R¹ or R² is hydrogen.

In another class of this sixth embodiment, R² is heteroaryl optionally substituted with one to three substituents independently selected from R^{b}.

In a seventh embodiment of the present invention, there are provided compounds of structural formula II having the indicated R stereochemical configuration at the stereogenic carbon atom marked with an *:

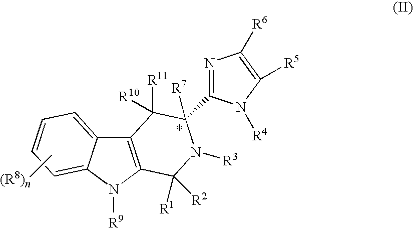

wherein R¹-R¹¹ and n are as defined above. In a class of this seventh embodiment, R³, R⁴, R⁵, R⁹, R¹⁰, and R¹¹ are each hydrogen; R⁷ is hydrogen or methyl; and n is 1. In a subclass of this class, R⁸ is hydrogen, halogen, or cyano.

In a second class of this seventh embodiment, R¹ is heteroaryl optionally substituted with one to three substituents independently selected from R^{b}, and R² is selected from the group consisting of:

hydrogen,

heteroaryl, optionally substituted with one to three substituents independently selected from R^{b},

C₁₋₃ alkyl-O—C₁₋₃ alkyl-, and

C₁₋₆ alkyl, wherein alkyl is optionally substituted with one to two substituents independently selected from R^{a}.

In a subclass of this class, R¹ or R² is hydrogen.

In a second subclass of this class, R² is heteroaryl optionally substituted with one to two substituents independently selected from R^{b}. In a subclass of this subclass, R¹ and R² are each independently heteroaryl selected from the group consisting of 1,2,4-oxadiazol-3-yl, 1,3,4-oxadiazol-2-yl, 1,2,4-thiadiazol-3-yl, pyrazol-3-yl, pyrazol-4-yl, 1,2,3-triazol-4-yl, 1,2,4-triazol-3-yl, 1,3-thiazol-4-yl, 1,3-thiazol-5-yl, and 1,3-oxazol-4-yl, each of which is optionally substituted with C₁₋₄ alkyl wherein alkyl is optionally substituted with one to five fluorines.

Illustrative, but nonlimiting examples, of the compounds of the present invention that are useful as antagonists of SSTR3 are the following beta-carbolines. Binding affinities for the SSTR3 receptor expressed as Kᵢ values are given below each structure.

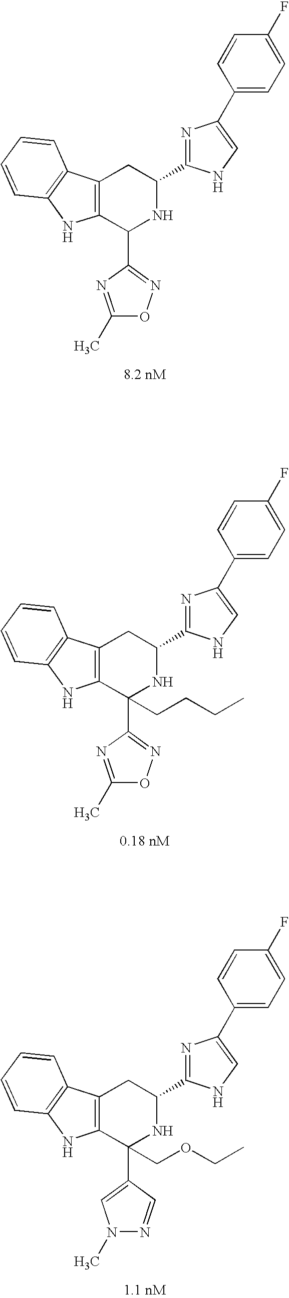

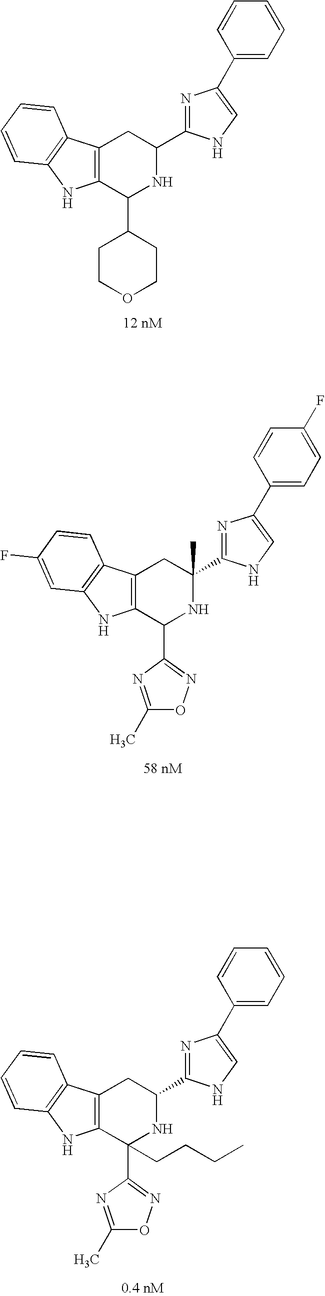

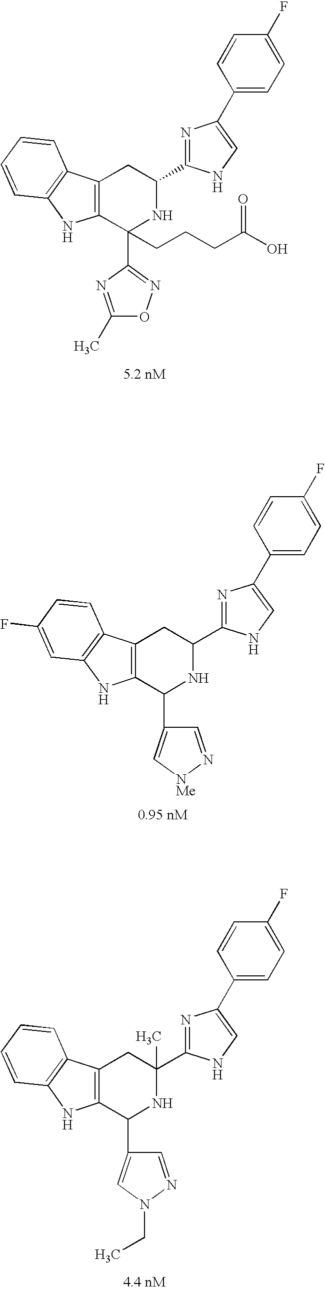

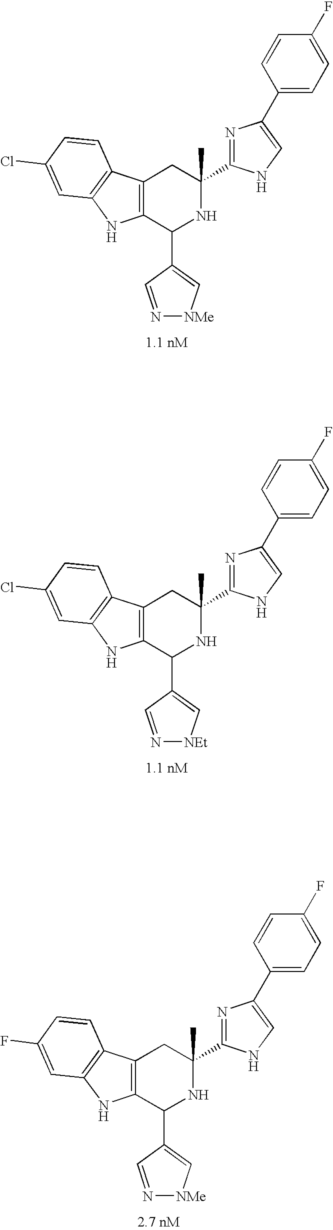

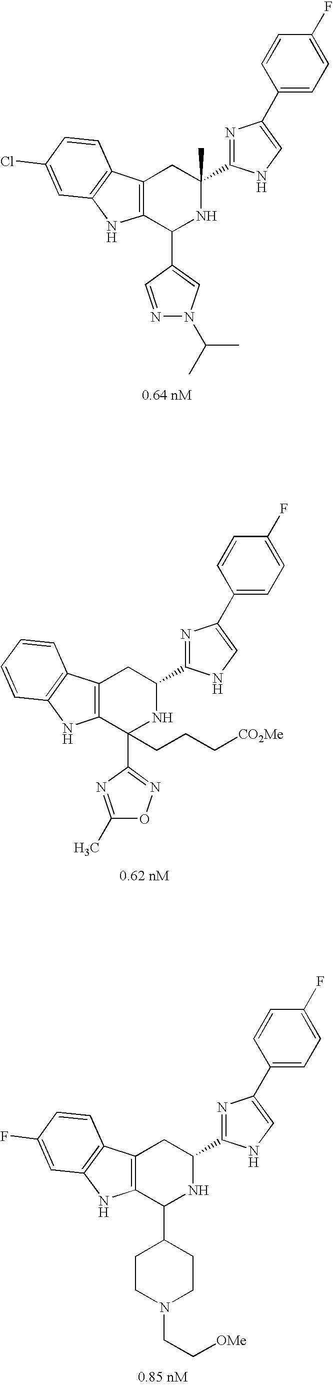

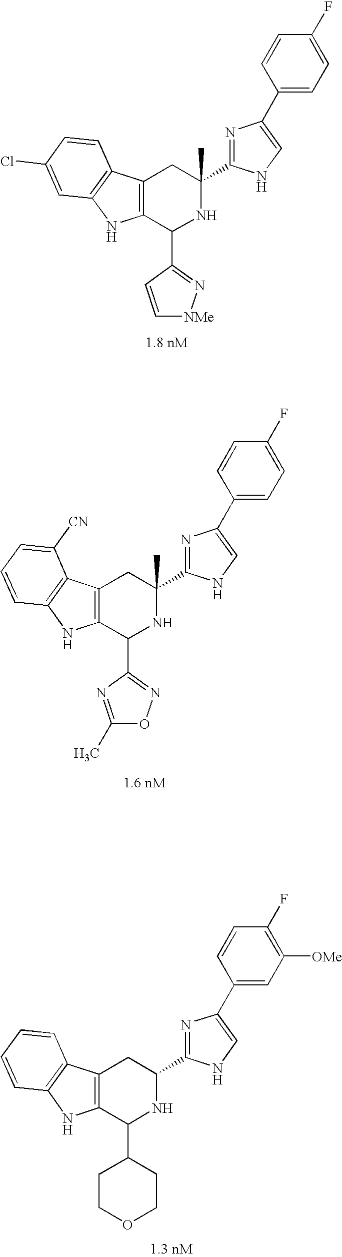

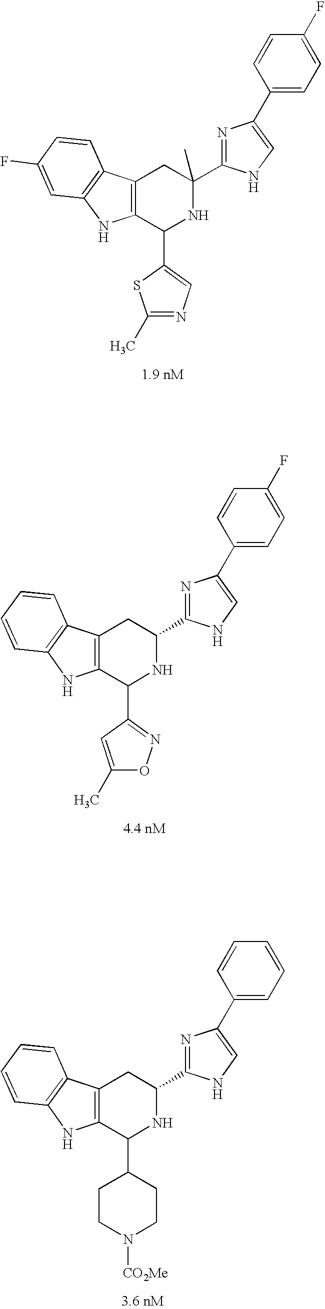

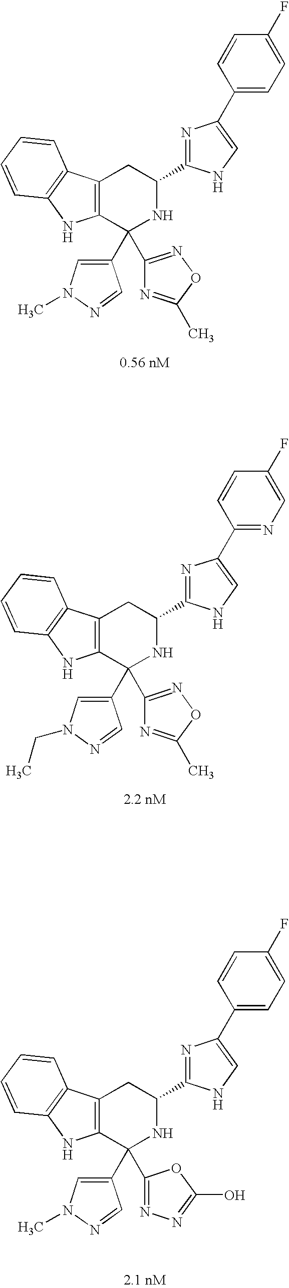

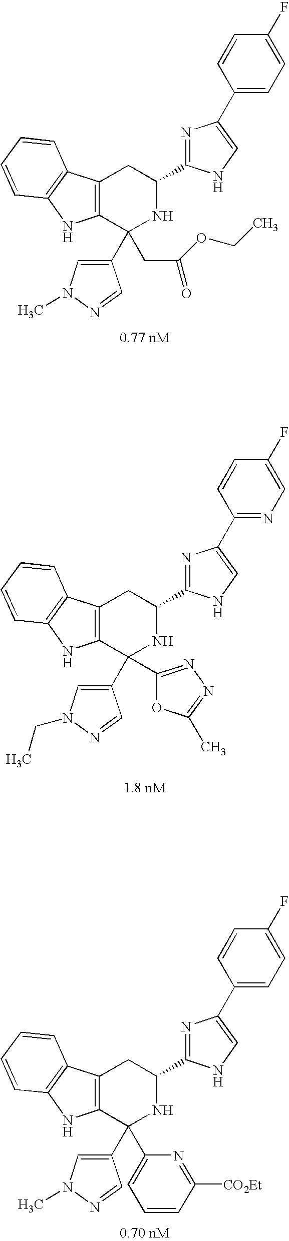

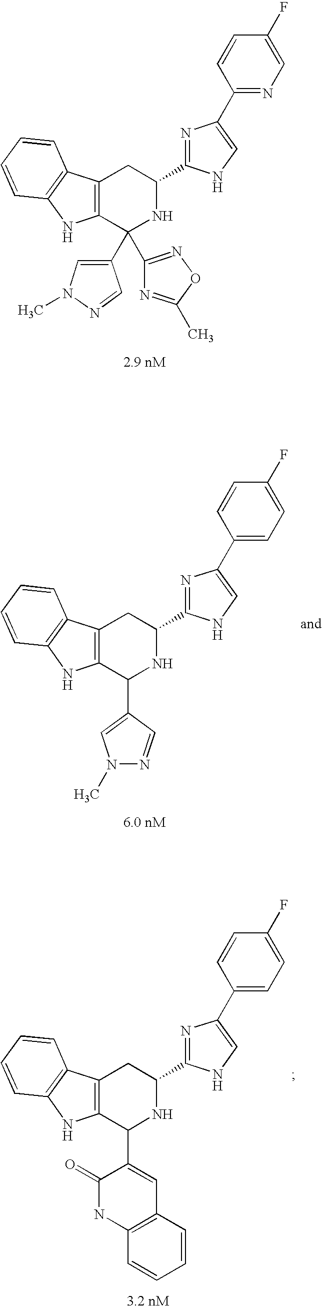

and pharmaceutically acceptable salts thereof.

Further illustrative of the compounds of the present invention that are useful as inhibitors of SSTR3 are the following:

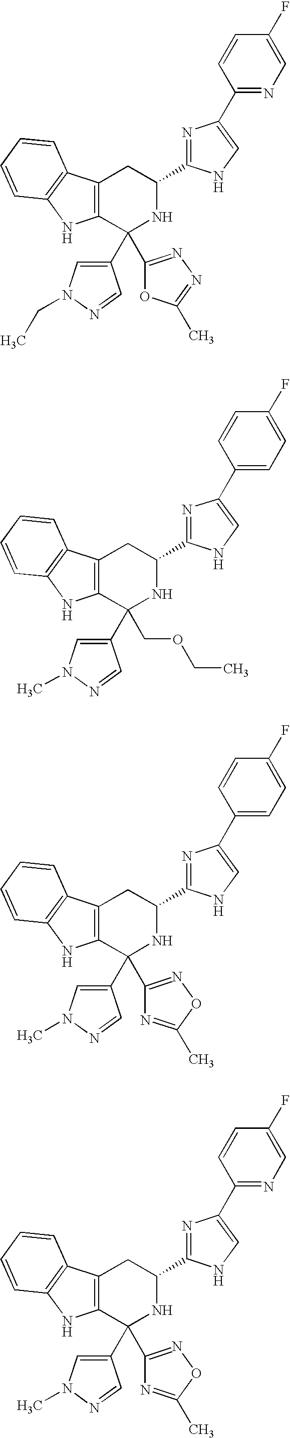

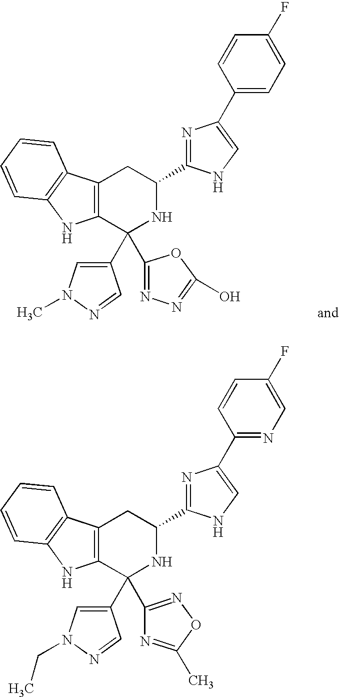

and pharmaceutically acceptable salts thereof.

The SSTR3 as identified herein is a target for affecting insulin secretion and assessing beta-cell mass. Glucose stimulated insulin secretion was found to be stimulated by abrogating the expression of SSTR3 and through the use of an SSTR3 selective antagonist. An important physiological action of insulin is to decrease blood glucose levels. As disclosed in the present application, targeting the SSTR3 has different uses including therapeutic applications, diagnostic applications, and evaluation of potential therapeutics.

Somatostatin is a hormone that exerts a wide spectrum of biological effects mediated by a family of seven transmembrane (TM) domain G-protein-coupled receptors [Lahlou et al., Ann. N.Y. Acad. Sci. 1014:121-131, 2004, Reisine et al., Endocrine Review 16:427-442, 1995]. The predominant active forms of somatostatin are somatostatin-14 and somatostatin-28. Somatostatin-14 is a cyclic tetradecapeptide. Somatostatin-28 is an extended form of somatostatin-14.

Somatostatin subtype receptor 3 (SSTR3) is the third, of five, related G-protein receptor subtypes responding to somatostatin. The other receptors are the somatostatin subtype receptor 1 (SSTR1), somatostatin subtype receptor 2 (SSTR2), somatostatin subtype receptor 4 (SSTR4) and somatostatin subtype receptor 5 (SSTR5). The five distinct subtypes are encoded by separate genes segregated on different chromosomes. (Patel et al., Neuroendocrinol. 20:157-198, 1999.) All five receptor subtypes bind somatostatin-14 and somatostatin-28, with low nanomolar affinity. The ligand binding domain for somatostatin is made up of residues in TMs III-VII with a potential contribution by the second extracellular loop. Somatostatin receptors are widely expressed in many tissues, frequently as multiple subtypes that coexist in the same cell.

The five different somatostatin receptors all functionally couple to inhibition of adenylate cyclase by a pertussin-toxin sensitive protein (Gₐᵢ₁₋₃) [Lahlou et al., Ann. N.Y. Acad. Sci. 1014:121-131, 2004]. Somatostatin-induced inhibition of peptide secretion results mainly from a decrease in intracellular Ca²⁺.

Among the wide spectrum of somatostatin effects, several biological responses have been identified with different receptor subtypes selectivity. These include growth hormone (GH) secretion mediated by SSTR2 and SSTR5, insulin secretion mediated by SSTR1 and SSTR5, glucagon secretion mediated by SSTR2, and immune responses mediated by SSTR2 [Patel et al., Neuroendocrinol. 20:157-198, 1999; Crider et al., Expert Opin. Ther. Patents 13:1427-1441, 2003].

Different somatostatin receptor sequences from different organisms are well known in the art. (See for example, Reisine et al., Endocrine Review 16:427-442, 1995.) Human, rat, and murine SSTR3 sequences and encoding nucleic acid sequences are provided in SEQ ID NO: 3 (human SSTR3 cDNA gi|44890055|ref|NM_{—}001051.2| CDS 526..1782); SEQ ID NO: 4 (human SSTR3AA gi|4557861|ref|NP_{—}001042.1|); SEQ ID NO: 5 (mouse SSTR3 cDNA gi|6678040|ref|NM_{—}009218.1| CDS 1..1287); SEQ ID NO: 6 (mouse SSTR3AA gi|6678041|ref|NP_{—}033244.1|); SEQ ID NO: 7 (rat SSTR3 cDNA gi|19424167|ref|NM_{—}133522.1| CDS 656..1942); SEQ ID NO: 8 (rat SSTR3A gi|19424168|ref|NP_{—}598206.1|).

SSTR3 antagonists can be identified using SSTR3 and nucleic acid encoding for SSTR3. Suitable assays include detecting compounds competing with a SSTR3 agonist for binding to SSTR3 and determining the functional effect of compounds on a SSTR3 cellular or physiologically relevant activity. SSTR3 cellular activities include cAMP inhibition, phospholipase C increase, tyrosine phsophatases increase, endothelial nitric oxide synthase (eNOS) decrease, K⁺ channel increase, Na⁺/H⁺ exchange decrease, and ERK decrease [Lahlou et al., Ann. N.Y. Acad. Sci. 1014:121-131, 2004]. Functional activity can be determined using cell lines expressing SSTR3 and determining the effect of a compound on one or more SSTR3 activities (e.g., Poitout et al., J. Med. Chem. 44: 2990-3000, 2001; Hocart et al., J. Med. Chem. 41:1146-1154, 1998).

SSTR3 binding assays can be performed by labeling somatostatin and determining the ability of a compound to inhibit somatostatin binding. (Poitout et al., J. Med. Chem. 44: 2990-3000, 2001; Hocart et al., J. Med. Chem. 41:1146-1154, 1998.) Additional formats for measuring binding of a compound to a receptor are well-known in the art.

A physiologically relevant activity for SSTR3 inhibition is stimulating insulin secretion. Stimulation of insulin secretion can be evaluated in vitro or in vivo.

SSTR3 antagonists can be identified experimentally or based on available information. A variety of different SSTR3 antagonists are well known in the art. Examples of such antagonists include peptide antagonists, β-carboline derivatives, and a decahydroisoquinoline derivative. [Poitout et al., J. Med. Chem. 44: 2990-3000 (2001), Hocart et al., J. Med. Chem. 41: 1146-1154 (1998), Reubi et al., PNAS 97:13973-13978 (2000), Banziger et al., Tetrahedron: Asymmetry 14: 3469-3477 (2003), Crider et al., Expert Opin. Ther. Patents 13:1427-1441 (2003), Troxler et al., International Publication No. WO 02/081471, International Publication Date Oct. 17, 2002].

Antagonists can be characterized based on their ability to bind to SSTR3 (Ki) and effect SSTR3 activity (IC₅₀), and to selectively bind to SSTR3 and selectively affect SSTR3 activity. Preferred antagonists strongly and selectively bind to SSTR3 and inhibit SSTR3 activity.

In different embodiments concerning SSTR3 binding, the antagonist has a Ki (nM) less than 100, preferably less than 50, more preferably less than 25 or more preferably less than 10. Ki can be measured as described by Poitout et al., J. Med. Chem. 44: 2990-3000 (2001) and described herein.

A selective SSTR3 antagonist binds SSTR3 at least 10 times stronger than it binds SSTR1, SSTR2, SSTR4, and SSTR5. In different embodiments concerning selective SSTR3 binding, the antagonist binds to each of SSTR1, SSTR2, SSTR4, and SSTR5 with a Ki greater than 1000, or preferably greater than 2000 nM and/or binds SSTR3 at least 40 times, more preferably at least 100 times, or more preferably at least 500 times, greater than it binds to SSTR1, SSTR2, SSTR4, and SSTR5.

In different embodiments concerning SSTR3 activity, the antagonist has an IC₅₀ (nM) less than 500, preferably less than 100, more preferably less than 50, or more preferably less than 10 nM. IC₅₀ can be determined by measuring inhibition of somatostatin-14 induced reduction of cAMP accumulation due to forskolin (1 μM) in CHO—K1 cells expressing SSTR3, as described by Poitout et al., J. Med. Chem. 44: 2990-3000, 2001.

Preferred antagonists have a preferred or more preferred Ki, a preferred or more preferred IC₅₀, and a preferred or more preferred selectivity. More preferred antagonists have a Ki (nM) less than 25; are at least 100 times selective for SSTR3 compared to SSTR1, SSTR2, SSTR4 and SSTR5; and have a IC₅₀ (nM) less than 50.

U.S. Pat. No. 6,586,445 discloses β-carboline derivatives as somatostatin receptor antagonists and sodium channel blockers denoted as being useful for the treatment of numerous diseases.

U.S. Pat. No. 6,861,430 also discloses β-carboline derivatives as SSTR3 antagonists for the treatment of depression, anxiety, and bipolar disorders.

Another set of examples are imidazolyl tetrahydro-β-carboline derivatives based on the compounds provided in Poitout et al., J. Med. Chem. 44:2990-3000, 2001.

Decahydroisoquinoline derivatives that are selective SSTR3 antagonists are disclosed in Banziger et al., Tetrahedron: Asymmetry 14:3469-3477, 2003.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic or bridged saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic cycloheteroalkyl group in which the point of attachment is on the aromatic portion. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means an aromatic or partially aromatic heterocycle that contains at least one ring heteroatom selected from O, S and N. “Heteroaryl” thus includes heteroaryls fused to other kinds of rings, such as aryls, cycloalkyls and heterocycles that are not aromatic. Examples of heteroaryl groups include pyrrolyl, isoxazolyl, isothiazolyl, pyrazolyl, pyridyl (pyridinyl), oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, 1,3-benzodioxolyl, benzo-1,4-dioxanyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Cycloheteroalkyl” means mono- or bicyclic or bridged saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “cycloheteroalkyl” include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. The cycloheteroalkyl ring may be substituted on the ring carbons and/or the ring nitrogens.

“Halogen” includes fluorine, chlorine, bromine and iodine.

By “oxo” is meant the functional group “═O”, such as, for example, (1) “C═(O)”, that is a carbonyl group; (2) “S═(O)”, that is, a sulfoxide group; and (3) “N═(O)”, that is, an N-oxide group, such as pyridyl-N-oxide.

When any variable (e.g., R¹, R^{a}, etc.) occurs more than one time in any constituent or in formula I, its definition on each occurrence is independent of its definition at every other occurrence. Also, combinations of substituents and/or variables are permissible only if such combinations result in stable compounds.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

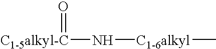

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

### Optical Isomers—Diastereoisomers—Geometric Isomers—Tautomers:

Compounds of structural formula I may contain one or more asymmetric centers and can thus occur as racemates and racemic mixtures, single enantiomers, diastereoisomeric mixtures and individual diastereoisomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention. Examples of tautomers which are intended to be encompassed within the compounds of the present invention are illustrated below:

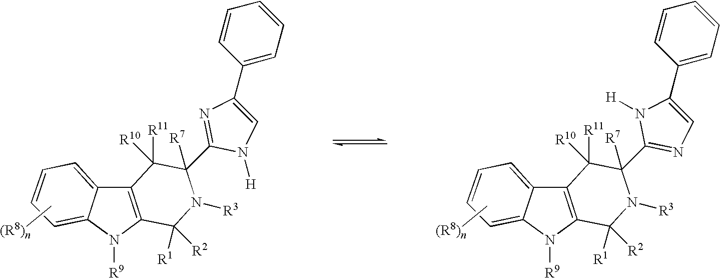

### Salts:

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Solvates, and in particular, the hydrates of the compounds of structural formula I are included in the present invention as well.

Exemplifying the invention is the use of the compounds disclosed in the Examples and herein.

### Utilities:

The compounds described herein are potent and selective antagonists of the somatostatin subtype receptor 3 (SSTR3). The compounds are efficacious in the treatment of diseases that are modulated by SSTR3 ligands, which are generally antagonists. Many of these diseases are summarized below.

One or more of the following diseases may be treated by the administration of a therapeutically effective amount of a compound of Formula I, or a pharmaceutically acceptable salt thereof, to a patient in need of treatment. Also, the compounds of Formula I may be used for the manufacture of a medicament for treating one or more of these diseases:

(1) non-insulin dependent diabetes mellitus (Type 2 diabetes);

(2) hyperglycemia;

(3) insulin resistance;

(4) Metabolic Syndrome;

(5) obesity;

(6) hypercholesterolemia;

(7) hypertriglyceridemia (elevated levels of triglyceride-rich-lipoproteins);

(8) mixed or diabetic dyslipidemia;

(9) low HDL cholesterol;

(10) high LDL cholesterol;

(11) hyper-apo-B lipoproteinemia; and

(12) atherosclerosis.

One embodiment of the uses of the compounds is directed to the treatment of one or more of the following diseases by administering a therapeutically effective amount to a patient, particularly a human, in need of treatment. The compounds may be used for manufacturing a medicament for use in the treatment of one or more of these diseases:

(1) Type 2 diabetes;

(2) hyperglycemia;

(3) insulin resistance;

(4) Metabolic Syndrome;

(5) obesity; and

(6) hypercholesterolemia.

The compounds are expected to be effective in lowering glucose and lipids in diabetic patients and in non-diabetic patients who have impaired glucose tolerance and/or are in a pre-diabetic condition. The compounds may ameliorate hyperinsulinemia, which often occurs in diabetic or pre-diabetic patients, by modulating the swings in the level of serum glucose that often occurs in these patients. The compounds may also be effective in treating or reducing insulin resistance. The compounds may be effective in treating or preventing gestational diabetes.

The compounds, compositions, and medicaments as described herein may also be effective in reducing the risks of adverse sequelae associated with Metabolic Syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

By keeping hyperglycemia under control, the compounds may also be effective in delaying or preventing vascular restenosis and diabetic retinopathy, neuropathy, and nephropathy.

The compounds of this invention may also have utility in improving or restoring β-cell function, so that they may be useful in treating type 1 diabetes or in delaying or preventing a patient with Type 2 diabetes from needing insulin therapy.

The compounds generally may be efficacious in treating one or more of the following diseases: (1) Type 2 diabetes (also known as non-insulin dependent diabetes mellitus, or NIDDM), (2) hyperglycemia, (3) impaired glucose tolerance, (4) insulin resistance, (5) obesity, (6) lipid disorders, (7) dyslipidemia, (8) hyperlipidemia, (9) hypertriglyceridemia, (10) hypercholesterolemia, (11) low HDL levels, (12) high LDL levels, (13) atherosclerosis and its sequelae, (14) vascular restenosis, (15) abdominal obesity, (16) retinopathy, (17) Metabolic Syndrome, (18) high blood pressure (hypertension), and (19) insulin resistance.

One aspect of the invention provides a method for the treatment and control of mixed or diabetic dyslipidemia, hypercholesterolemia, atherosclerosis, low HDL levels, high LDL levels, hyperlipidemia, and/or hypertriglyceridemia, which comprises administering to a patient in need of such treatment a therapeutically effective amount of a compound having formula I. The compound may be used alone or advantageously may be administered with a cholesterol biosynthesis inhibitor, particularly an HMG-CoA reductase inhibitor such as lovastatin, simvastatin, rosuvastatin, pravastatin, fluvastatin, atorvastatin, rivastatin, itavastatin, or ZD-4522. The compound may also be used advantageously in combination with other lipid lowering drugs such as cholesterol absorption inhibitors (for example stanol esters, sterol glycosides such as tiqueside, and azetidinones, such as ezetimibe), ACAT inhibitors (such as avasimibe), CETP inhibitors (such as torcetrapib and those described in published applications WO2005/100298, WO2006/014413, and WO2006/014357), niacin and niacin receptor agonists, bile acid sequestrants, microsomal triglyceride transport inhibitors, and bile acid reuptake inhibitors. These combination treatments may be effective for the treatment or control of one or more related conditions selected from the group consisting of hypercholesterolemia, atherosclerosis, hyperlipidemia, hypertriglyceridemia, dyslipidemia, high LDL, and low HDL.

### Administration and Dose Ranges:

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus and/or hyperglycemia or hypertriglyceridemia or other diseases for which compounds of Formula I are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as one gm. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 25 mg, 50 mg, 100 mg, 200 mg, 300 mg, 400 mg, 500 mg, and 750 mg. Other oral forms may also have the same or similar dosages.

### Pharmaceutical Compositions:

Another aspect of the present invention provides pharmaceutical compositions which comprise a compound of Formula I and a pharmaceutically acceptable carrier. The pharmaceutical compositions of the present invention comprise a compound of Formula I or a pharmaceutically acceptable salt as an active ingredient, as well as a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids. A pharmaceutical composition may also comprise a prodrug, or a pharmaceutically acceptable salt thereof, if a prodrug is administered.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

In some instances, depending on the solubility of the compound or salt being administered, it may be advantageous to formulate the compound or salt as a solution in an oil such as a triglyceride of one or more medium chain fatty acids, a lipophilic solvent such as triacetin, a hydrophilic solvent (e.g. propylene glycol), or a mixture of two or more of these, also optionally including one or more ionic or nonionic surfactants, such as sodium lauryl sulfate, polysorbate 80, polyethoxylated triglycerides, and mono and/or diglycerides of one or more medium chain fatty acids. Solutions containing surfactants (especially 2 or more surfactants) will form emulsions or microemulsions on contact with water. The compound may also be formulated in a water soluble polymer in which it has been dispersed as an amorphous phase by such methods as hot melt extrusion and spray drying, such polymers including hydroxylpropylmethylcellulose acetate (HPMCAS), hydroxylpropylmethyl cellulose (HPMCS), and polyvinylpyrrolidinones, including the homopolymer and copolymers.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### Combination Therapy:

Compounds of Formula I may be used in combination with other drugs that may also be useful in the treatment or amelioration of the diseases or conditions for which compounds of Formula I are useful. Such other drugs may be administered, by a route and in an amount commonly used therefor, contemporaneously or sequentially with a compound of Formula I. In the treatment of patients who have Type 2 diabetes, insulin resistance, obesity, Metabolic Syndrome, and co-morbidities that accompany these diseases, more than one drug is commonly administered. The compounds of this invention may generally be administered to a patient who is already taking one or more other drugs for these conditions. Often the compounds will be administered to a patient who is already being treated with one or more antidiabetic compound, such as metformin, sulfonylureas, and/or PPAR gamma agonists, when the patient's glycemic levels are not adequately responding to treatment.

When a compound of Formula I is used contemporaneously with one or more other drugs, a pharmaceutical composition in unit dosage form containing such other drugs and the compound of Formula I is preferred. However, the combination therapy also includes therapies in which the compound of Formula I and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of Formula I, and either administered separately or in the same pharmaceutical composition, include, but are not limited to:

(a) PPAR gamma agonists and partial agonists, including both glitazones and non-glitazones (e.g., troglitazone, pioglitazone, englitazone, MCC-555, rosiglitazone, balaglitazone, netoglitazone, T-131, LY-300512, LY-818, and compounds disclosed in WO02/08188, WO2004/020408, and WO2004/020409.

(b) biguanides, such as metformin and pharmaceutically acceptable salts thereof;

(c) protein tyrosine phosphatase-1B (PTP-1B) inhibitors;

(d) dipeptidyl peptidase-IV (DPP-4) inhibitors;

(e) insulin or insulin mimetics;

(f) oral hypoglycemic sulfonylurea drugs, such as tolbutamide, glyburide, glimepiride, glipizide, and related materials;

(g) α-glucosidase inhibitors (such as acarbose);

(h) agents which improve a patient's lipid profile, such as (i) HMG-CoA reductase inhibitors (lovastatin, simvastatin, rosuvastatin, pravastatin, fluvastatin, atorvastatin, rivastatin, itavastatin, ZD-4522 and other statins), (ii) bile acid sequestrants (cholestyramine, colestipol, and dialkylaminoalkyl derivatives of a cross-linked dextran), (iii) niacin receptor agonists, nicotinyl alcohol, nicotinic acid, or a salt thereof, (iv) PPARα agonists, such as fenofibric acid derivatives (gemfibrozil, clofibrate, fenofibrate and bezafibrate), (v) cholesterol absorption inhibitors, such as ezetimibe, (vi) acyl CoA:cholesterol acyltransferase (ACAT) inhibitors, such as avasimibe, (vii) CETP inhibitors, such as torcetrapib, and (viii) phenolic antioxidants, such as probucol;

(i) PPARα/γ dual agonists, such as muraglitazar, tesaglitazar, farglitazar, and JT-501;

(j) PPARδ agonists, such as those disclosed in WO97/28149;

(k) anti-obesity compounds, such as fenfluramine, dexfenfluramine, phentiramine, subitramine, orlistat, neuropeptide Y Y5 inhibitors, MC4R agonists, cannabinoid receptor 1 (CB-1) antagonists/inverse agonists (e.g., rimonabant and taranabant), and β₃ adrenergic receptor agonists;

(l) ileal bile acid transporter inhibitors;

(m) agents intended for use in inflammatory conditions, such as aspirin, non-steroidal anti-inflammatory drugs, glucocorticoids, azulfidine, and cyclooxygenase-2 (Cox-2) selective inhibitors;

(n) glucagon receptor antagonists;

(o) GLP-1 analogs and derivatives, such as exendins (e.g., exenatide and liruglatide);

(p) inhibitors of 11β-hydroxysteroid dehydrogenase type 1, such as those disclosed in U.S. Pat. No. 6,730,690; WO 03/104207; and WO 04/058741;

(q) stearoyl-coenzyme A delta 9 desaturase (SCD) inhibitors;

(r) glucagon receptor antagonists;

(s) glucokinase activators (GKAs), such as those disclosed in WO 03/015774; WO 04/076420; and WO 04/081001;

(t) AMPK activators;

(u) antihypertensive agents, such as ACE inhibitors (enalapril, lisinopril, captopril, quinapril, tandolapril), A-H receptor blockers (losartan, candesartan, irbesartan, valsartan, telmisartan, and eprosartan), beta blockers and calcium channel blockers;

(v) G-protein coupled receptor-40 agonists, such as those disclosed in WO 2008/054674 and WO 2008/054675; and

(w) G-protein coupled receptor-119 antogonists.

The above combinations include combinations of a compound of the present invention not only with one other active compound, but also with two or more other active compounds. Non-limiting examples include combinations of compounds having Formula I with two or more active compounds selected from metformin, sulfonylureas, HMG-CoA reductase inhibitors, PPAR gamma agonists, DPP-4 inhibitors, and cannabinoid receptor 1 (CB1) inverse agonists/antagonists.

The preferred pharmaceutically aceptable salt of metformin is the hydrochloride salt. The metformin compoent in the combination may be either formulated for either immediate release, such as Glucophage™, or for extended-release, such as Glucophage XR™, Glumetza™ and Fortamet™.

Dipeptidyl peptidase-IV (DPP-4) inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-IV inhibitor compounds include sitagliptin (JANUVIA™); vildagliptin (GALVUS™); denagliptin; P93/01; saxagliptin (BMS 477118); RO0730699; MP513; alogliptin (SYR-322); ABT-279; PHX1149; GRC-8200; TS021; and pharmaceutically acceptable salts thereof.

Antiobesity compounds that can be combined with compounds of structural formula I include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474, and WO 03/104761.

Another aspect of the present invention relates to methods for the treatment of Type 2 diabetes, hyperglycemia, insulin resistance, and obesity with a therapeutically effective amount of an SSTR3 antagonist in combination with a therapeutically effective amount of a dipeptidyl peptidase-IV (DPP-4) inhibitor. In one embodiment of this aspect of the present invention the DPP-4 inhibitor is selected from the group consisting of sitagliptin, vildagliptin, saxagliptin, alogliptin, denagliptin, and melogliptin, and pharmaceutically acceptable salts thereof.

A particular pharmaceutically acceptable salt of sitagliptin is sitagliptin phosphate having structural formula I below which is the dihydrogenphosphate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

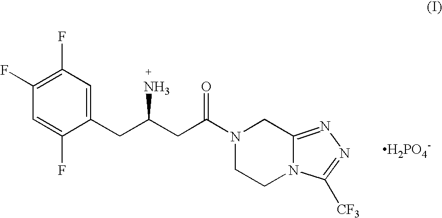

In one embodiment sitagliptin phosphate is in the form of a crystalline anhydrate or monohydrate. In a class of this embodiment, sitagliptin phosphate is in the form of a crystalline monohydrate. Sitagliptin free base and pharmaceutically acceptable salts thereof are disclosed in U.S. Pat. No. 6,699,871, the contents of which are hereby incorporated by reference in their entirety. Sitagliptin phosphate and a crystalline monohydrate form is disclosed in U.S. Pat. No. 7,326,708, the contents of which are hereby incorporated by reference in their entirety.

Vildagliptin is the generic name for (S)-1-[(3-hydroxy-1-adamantypamino]acetyl-2-cyano-pyrrolidine having structural formula II. Vildagliptin is specifically disclosed in U.S. Pat. No. 6,166,063, the contents of which are hereby incorporated by reference in their entirety.

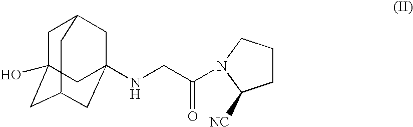

Saxagliptin is a methanoprolinenitrile of structural formula III below. Saxagliptin is specifically disclosed in U.S. Pat. No. 6,395,767, the contents of which are hereby incorporated by reference in their entirety.

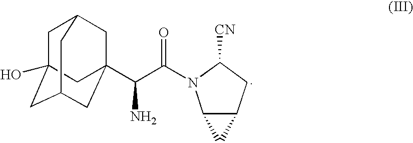

Alogliptin is 2-[[6-[(3R)-3-amino-1-piperidinyl]3,4-dihydro-3-methyl-2,4-dioxo-1(2H)-pyrimidinyl]methyl]benzonitrile of structural formula (IV) which is disclosed in US 2005/0261271. A particular pharmaceutically acceptable salt of alogliptin is alogliptin benzoate.

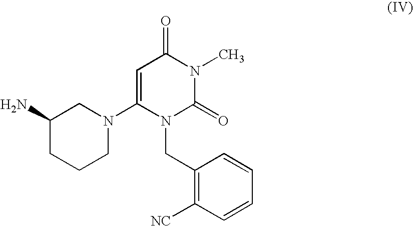

Yet a another aspect of the present invention is a combination of an SSTR3 antagonist and a DPP-4 inhibitor. In one embodiment the DPP-4 inhibitor is selected from the group consisting of sitagliptin, vildagliptin, saxagliptin, alogliptin, denagliptin, and melogliptin, and pharmaceutically acceptable salts thereof. In a class of this embodiment the DPP-4 inhibitor is sitagliptin or a pharmaceutically acceptable salt thereof. This combination is useful for the treatment of Type diabetes, hyperglycemia, insulin resistance, and obesity.

### Biological Assays

### Somatostatin Subtype Receptor 3 Production

SSTR3 can be produced using techniques well known in the art including those involving chemical synthesis and those involving recombinant production [See e.g., Vincent, Peptide and Protein Drug Delivery, New York, N.Y., Decker, 1990; Current Protocols in Molecular Biology, John Wiley, 1987-2002, and Sambrook et al., Molecular Cloning, A Laboratory Manual, 2^{nd} Edition, Cold Spring Harbor Laboratory Press, 1989].

Recombinant nucleic acid techniques for producing a protein involve introducing, or producing, a recombinant gene encoding the protein in a cell and expressing the protein. A purified protein can be obtained from cell. Alternatively, the activity of the protein in a cell or cell extract can be evaluated.

A recombinant gene contains nucleic acid encoding a protein along with regulatory elements for protein expression. The recombinant gene can be present in a cellular genome or can be part of an expression vector.

The regulatory elements that may be present as part of a recombinant gene include those naturally associated with the protein encoding sequence and exogenous regulatory elements not naturally associated with the protein encoding sequence. Exogenous regulatory elements such as an exogenous promoter can be useful for expressing a recombinant gene in a particular host or increasing the level of expression. Generally, the regulatory elements that are present in a recombinant gene include a transcriptional promoter, a ribosome binding site, a terminator, and an optionally present operator. A preferred element for processing in eukaryotic cells is a polyadenylation signal.

Expression of a recombinant gene in a cell is facilitated through the use of an expression vector. Preferably, an expression vector in addition to a recombinant gene also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

If desired, expression in a particular host can be enhanced through codon optimization. Codon optimization includes use of more preferred codons. Techniques for codon optimization in different hosts are well known in the art.

### Enhancement of Glucose Dependent Insulin Secretion (GDIS) by SSTR3 Antagonists in Isolated Mouse Islet Cells:

Pancreatic islets of Langerhans were isolated from the pancreas of normal C57BL/6J mice (Jackson Laboratory, Maine) by collagenase digestion and discontinuous Ficoll gradient separation, a modification of the original method of Lacy and Kostianovsky (Lacy et al., Diabetes 16:35-39, 1967). The islets were cultured overnight in RPMI 1640 medium (11 mM glucose) before GDIS assay.

To measure GDIS, islets were first preincubated for 30 minutes in the Krebs-Ringer bicarbonate (KRB) buffer with 2 mM glucose (in petri dishes). The KRB medium contains 143.5 mM Na⁺, 5.8 mM K⁺, 2.5 mM Ca²⁺, 1.2 mM Mg²⁺, 124.1 mM Cl⁻, 1.2 mM PO₄³⁻, 1.2 mM SO₄²⁺, 25 mM CO₃²⁻, 2 mg/mL bovine serum albumin (pH 7.4). The islets were then transferred to a 96-well plate (one islet/well) and incubated at 37° C. for 60 min in 200 μL of KRB buffer with 2 or 16 mM glucose, and other agents to be tested such as octreotide and a SSTR3 antagonist. (Zhou et al., J. Biol. Chem. 278:51316-51323, 2003.) Insulin was measured in aliquots of the incubation buffer by ELISA with a commercial kit (ALPCO Diagnostics, Windham, N.H.).

### SSTR Binding Assays:

The receptor-ligand binding assays of all 5 subtype of SSTRs were performed with membranes isolated from Chinese hamster ovary (CHO)—K1 cells stably expressing the cloned human somatostatin receptors in 96-well format as previous reported. (Yang et al. PNAS 95:10836-10841, 1998, Birzin et al. Anal. Biochem. 307:159-166, 2002.)

The stable cell lines for SSTR1-SSTR5 were developed by stably transfecting with DNA for all five SSTR's using Lipofectamine. Neomycin-resistant clones were selected and maintained in medium containing 400 μg/mL G418 (Rohrer et al. Science 282:737-740, 1998). Binding assays were performed using (3-¹²⁵I-Tyr11)-SRIF-14 as the radioligand (used at 0.1 nM) and The Packard Unifilter assay plate. The assay buffer consisted of 50 mM TrisHCl (pH 7.8) with 1 mM EGTA, 5 mM MgCl₂, leupeptin (10 μg/mL), pepstatin (10 μg/mL), bacitracin (200 μg/mL), and aprotinin (0.5 μg/mL). CHO—K1 cell membranes, radiolabeled somatostatin, and unlabeled test compounds were resuspended or diluted in this assay buffer. Unlabeled test compounds were examined over a range of concentrations from 0.01 nM to 10,000 nM. The Kᵢ values for compounds were determined as described by Cheng and Prusoff, Biochem Pharmacol. 22:3099-3108 (1973).

Compounds of the present invention, particularly the compounds of Examples 1-19 and the Examples listed in Tables 2-5, exhibited Kᵢ values in the range of 100 nM to 0.1 nM against SSTR3 and exhibited Kᵢ values greater than 100 nM against SSTR1, SSTR2, SSTR4, and SSTR5 receptors.

### Functional Assay to Assess the Inhibition of SSTR3 Mediated Cyclic AMP Production:

The effects of compounds that bind to human and murine SSTR3 with various affinities on the functional activity of the receptor were assessed by measuring cAMP production in the presence of Forskolin (FSK) along or FSK plus SS-14 in SSTR3 expressing CHO cells. FSK acts to induce cAMP production in these cells by activating adenylate cyclases, whereas SS-14 suppresses cAMP production in the SSTR3 stable cells by binding to SSTR3 and the subsequent inhibition of adenylate cyclases via an alpha subunit of GTP-binding protein (Gαi).

To measure the agonism activity of the compounds, we pre-incubated the human or mouse SSTR3 stable CHO cells with the compounds for 15 min, followed by a one-hour incubation of the cells with 3.5 μM FSK (in the continuous presence of the compounds). The amount of cAMP produced during the incubation was quantified with the Lance cAMP assay kit (PerkinElmer, CA) according to the manufacturer's instruction. Majority of the compounds described in this application show no or little agonism activity. Therefore we used % Activation to reflect the agonism activity of each compound. The % Activation which was calculated with the following formula:

% Activation=[(FSK−Unknown)/(FSK−SS-14]×100

To measure the antagonism activity of the compounds, we pre-incubated the human or mouse SSTR3 stable CHO cells with the compounds for 15 min, followed by a one-hour incubation of the cells with a mixture of 3.5 μM FSK+100 nM SS-14 (in the continuous presence of the compounds). The amount of cAMP produced during the incubation was also quantified with the Lance cAMP assay. The antagonism activity of each compound was reflected both by % Inhibition (its maximum ability to block the action of SS-14) and an IC₅₀ value obtained by a eight-point titration. The % Inhibition of each compound was calculated using the following formula:

% Inhibition=[1−(unknown cAMP/FSK+SS-14 cAMP)]×100

In some case, 20% of human serum was included in the incubation buffer during the antagonism mode of the function assay to estimate the serum shift of the potency.

### Glucose Tolerance Test in Mice:

Male C57BL/6N mice (7-12 weeks of age) are housed 10 per cage and given access to normal diet rodent chow and water ad libitum. Mice are randomly assigned to treatment groups and fasted 4 to 6 h. Baseline blood glucose concentrations are determined by glucometer from tail nick blood. Animals are then treated orally with vehicle (0.25% methylcellulose) or test compound. Blood glucose concentration is measured at a set time point after treatment (t=0 min) and mice are then challenged with dextrose intraperitoneally- (2-3 g/kg) or orally (3-5 g/kg). One group of vehicle-treated mice is challenged with saline as a negative control. Blood glucose levels are determined from tail bleeds taken at 20, 40, 60 minutes after dextrose challenge. The blood glucose excursion profile from t=0 to t=60 min is used to integrate an area under the curve (AUC) for each treatment. Percent inhibition values for each treatment are generated from the AUC data normalized to the saline-challenged controls. A similar assay may be performed in rats. Compounds of the present invention are active after an oral dose in the range of 0.1 to 100 mg/kg.

### Glucose Tolerance Test in SSTR3 Gene Knockout Mice:

In order to assess the selectivity of blockade of SSTR3, compounds were evaluated in the oral glucose tolerance test (oGTT) described above in mice lacking the gene for a functional SSTR3. Whereas Examples 17, 20, and 21 inhibit glucose excursion in wild type mice containing intact, functional SSTR3, they failed to significantly inhibit glucose excursion in the SSTR3 knock out mice after an oral dose in the range of 1 to 30 mg/kg po.

### Abbreviations Used in the Following Schemes and Examples:

AcOH: acetic acid
Ac₂O: acetic anhydride
aq.: aqueous
API-ES: atmospheric pressure ionization-electrospray (mass spectrum term)
AcCN: acetonitrile
Boc: tert-butyloxycarbonyl
d: day(s)
DCM: dichloromethane
DEAD: diethyl azodicarboxylate
MAL: di-isobutylaluminum hydride
DIPEA: N,N-diisopropylethylamine (Hunig's base)
DMAP: 4-dimethylaminopyridine

### DMF: N,N-dimethylformamide

DMSO: dimethylsulfoxide
EDC: 1-ethyl-3-(3-dimethylaminopropyl)-carbodiimide hydrochloride
EPA: ethylene polyacrylamide (a plastic)
EtOAc: ethyl acetate
Et: ethyl
g or gm: gram
h or hr: hour(s)
Hex: hexane
HOBt: 1-hydroxybenzotriazole
HPLC: high pressure liquid chromatography
HPLC/MS: high pressure liquid chromatography/mass spectrum
in vacuo: rotary evaporation under diminished pressure
IPA: isopropyl alcohol
IPAC or IPAc: isopropyl acetate
KHMDS: potassium hexamethyldisilazide
L: liter
LC: Liquid chromatography
LC-MS: liquid chromatography-mass spectrum
LDA: lithium diisopropylamide
M: molar
Me: methyl
MeOH: methanol
MHz: megahertz
mg: milligram
min: minute(s)
mL: milliliter
mmol: millimole
MPLC: medium-pressure liquid chromatography
MS or ms: mass spectrum
MTBE: methyl tert-butyl ether
N: normal
NaHMDS: sodium hexamethyldisilazide
nOe: nuclear Overhauser effect
nm: nanometer
nM: nanomolar
NMR: nuclear magnetic resonance

### NMM: N-methylmorpholine

OD: octadecyl (C₁₈)
PrepTLC: preparative thin layer chromatography
PyBOP: (benzotriazol-1-yloxy)tripyrrolidinophosphonium hexafluorophosphate
Rₜ: retention time
rt or RT: room temperature
SFC: supercritical fluid chromatography
TEA: triethylamine
TFA: trifluoroacetic acid
TFAA: trifluoroacetic acid anhydride
THF: tetrahydrofuran
TLC or tlc: thin layer chromatography

Several methods for preparing the compounds of this invention are illustrated in the following Schemes and Examples. Starting materials are either commercially available or made by known procedures in the literature or as illustrated. The present invention further provides processes for the preparation of compounds of structural formula I as defined above. In some cases the order of carrying out the foregoing reaction schemes may be varied to facilitate the reaction or to avoid unwanted reaction products. The following examples are provided for the purpose of illustration only and are not to be construed as limitations on the disclosed invention. All temperatures are degrees Celsius unless otherwise noted. The assignment of stereochemistry at the stereogenic carbon center indicated by an ** in Structure G of Scheme 3 from the Pictet-Spengler cyclization reaction to elaborate the β-carboline nucleus was determined using the aid of nuclear Overhauser effect (NOE) NMR spectroscopy. For a thorough discussion of the theory and application of NOE NMR spectroscopy, reference is made to Ernst, R. R.; Bodenhausen, B.; Wokaun, A., “Principles of Nuclear Magnetic Resonances in One or Two Dimensions”, Oxford University Press, 1992; Neuhaus, D.; Williamson, M. P., “The Nuclear Overhauser Effect in Structural and Conformational Analysis, 2^{nd} Edition”, in “Methods in Stereochemical Analysis”, Marchand, A. P. (series editor), John A. Wiley and Sons, New York 2000.

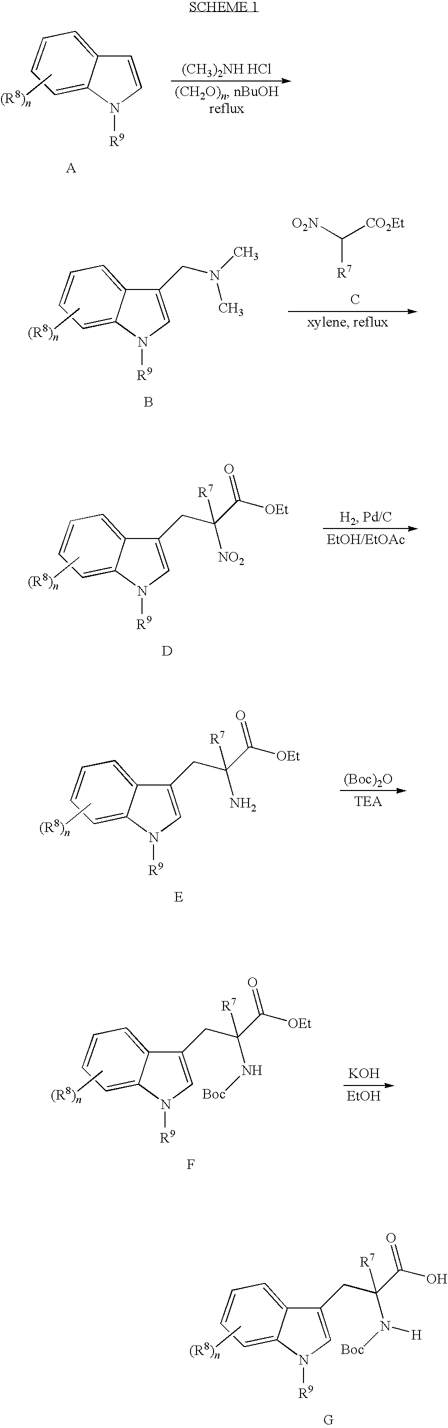

In Scheme 1, substituted indoles A are treated with dimethylamine and paraformaldehyde in a Mannich reaction to form 3-(dimethylamino)methyl-indole B. Reaction of B with nitro ester C affords the 3-(indol-3-yl)-2-nitro-propionic acid, ethyl ester D which is reduced to tryptophan derivative E. Acylation of the amine in E and hydrolysis of the ester F affords the appropriately protected tryptophan derivative G. Separation of the isomers of F or G by chiral column chromatography yields the individual enantiomers.

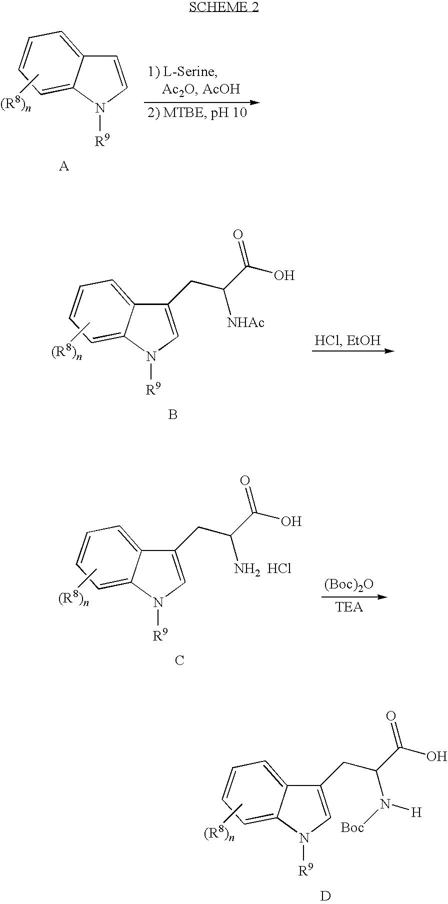

In Scheme 2, substituted indole A is reacted with L-serine in the presence of acetic anhydride and acetic acid to form tryptophan B. Hydrolysis of the amide followed by amine protection affords the desired substituted tryptophan intermediate D.

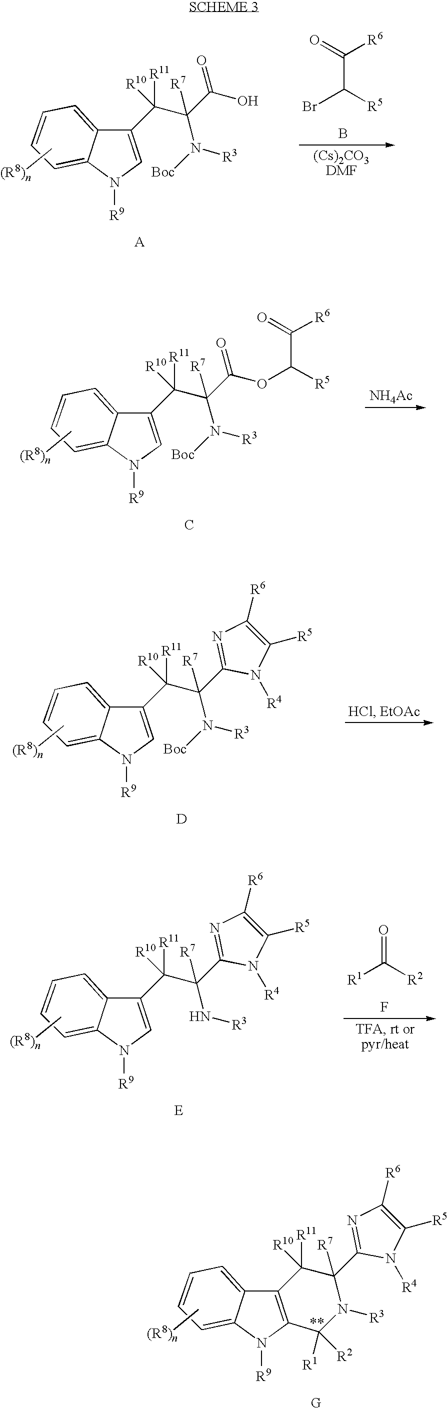

In Scheme 3, substituted tryptophan derivative A is reacted with α-bromo-ketone B to afford ester C. Reaction with ammonium acetate effects cyclization to form substituted imidazole D. Removal of the N-Boc protecting group with acid yields indole imidazole E which is reacted with aldehydes or ketones F in a Pictet-Spengler cyclization to afford the desired product G.

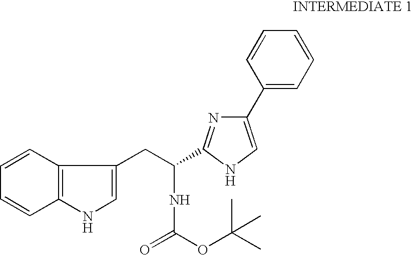

### tert-Butyl (1R)-2-(1H-indol-3-yl)-1-(4-phenyl-1H-imidazol-2-yl)-1-ethylcarbamate

The title compound was prepared from N-Boc-D-tryptophan and 2-bromoacetophenone by methods described in the literature (Gordon, T. et al., Bioorg. Med. Chem. Lett. 1993, 3, 915; Gordon, T. et al., Tetrahedron Lett. 1993, 34, 1901; Poitout, L. et al., J. Med. Chem. 2001, 44, 2990).

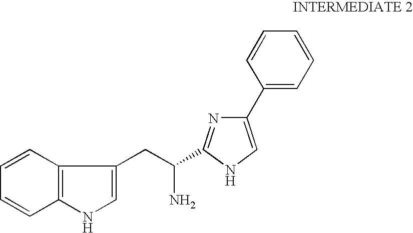

### (1R)-2(1H-Indol-3-yl)-1-(4-phenyl-1H-imidazol-2-yl)-1-ethanamine

The title compound was prepared from tert-butyl (1R)-2(1H-indol-3-yl)-1-(4-phenyl-1H-imidazol-2-yl)-1-ethylcarbamate by treatment with hydrochloric acid or trifluoroacetic acid according to the methods described in the literature (Gordon, T. et al., Bioorg. Med. Chem. Lett. 1993, 3, 915; Gordon, T. et al., Tetrahedron Lett. 1993, 34, 1901; Poitout, L. et al., J. Med. Chem. 2001, 44, 2990).

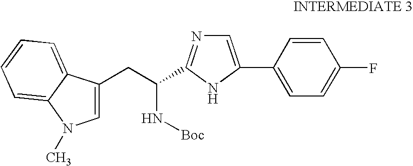

### tert-Butyl (1R)-2-(1-methyl-1H-indol-3-yl)-1-(4-(4-fluorophenyl)-1H-imidazol-2-yl)ethylcarbamate

### Step A: N^{α}-tert-Butyloxycarbonyl-1-methyl-D-tryptophan

A 100 mL one-neck round bottom flask was charged with 1-methyl-D-tryptophan (3.4 g, 15.58 mmol), methanol (50 mL), and DIPEA (4.03 g, 31.2 mmol). The mixture was stirred while di-tert-butyl dicarbonate (4.08 g, 18.69 mmol) was added and until all the solid was dissolved. The mixture was then stirred for 30 min. The solvent was removed by rotary evaporation and the residue was partitioned between ethyl acetate (30 mL) and 1N HCl (15 mL). The aqueous layer was adjusted to pH=4. The organic layer was separated and the aqueous layer was extracted three times with ethyl acetate. The combined organic phases were washed with brine, dried over MgSO₄, filtered and concentrated to afford crude N^{α}-tert-butyloxycarbonyl-1-methyl-D-tryptophan which was used directly in the next step without further purification. LC-MS: m/z 319 (M+H)' (3.0 min).

### Step B: N-(tert-Butoxycarbonyl)-1-methyl-D-tryptophan, 2-(4-fluorophenyl)-2-oxoethyl ester

A 100 mL one-neck round bottom flask was charged N^{α}-tert-butyloxycarbonyl-1-methyl-D-tryptophan (4.96 g, 15.58 mmol), cesium carbonate (2.69 g, 8.26 mmol) and ethanol (40 mL). The mixture was stirred at rt for 30 min and the solvent was removed by rotary evaporation. To the resulting salt in DMF (40 mL) was added 2-bromo-4′-fluoroacetophenone (3.45 g, 15.89 mmol). The mixture was stirred at rt under nitrogen for 18 h. The solvent was removed by rotary evaporation and the residue was diluted with ethyl acetate (100 mL). The CsBr was filtered and washed with ethyl acetate (50 mL). The filtrate was concentrated to afford N-(tert-butoxycarbonyl)-1-methyl-D-tryptophan, 2-(4-fluorophenyl)-2-oxoethyl ester which was used directly in the next step without further purification. LC-MS: m/z 455 (M+H)⁺ (1.25 min).

### Step C: tert-Butyl (1R)-2-(1-methyl-1H-indol-3-yl)-1-(4-(4-fluorophenyl)-1H-imidazol-2-yl)ethylcarbamate

A 200 mL one-neck round bottom flask was charged with N-(tert-butoxycarbonyl)-1-methyl-D-tryptophan, 2-(4-fluorophenyl)-2-oxoethyl ester (7.08 g, 15.58 mmol), ammonium acetate (4.80 g, 62.3 mmol) and xylene (40 mL). The mixture was then heated at reflux temperature for 3 h. After cooling to rt, the mixture was diluted with ethyl acetate (100 mL) and then washed with water, saturated aqueous NaHCO₃, brine, dried over MgSO₄, filtered and concentrated. The crude product was purified by MPLC (120 g silica gel, 0 to 40% ethyl acetate in hexanes as the mobile phase) to afford tert-butyl 1(R)-2-(1-methyl-1H-indol-3-yl)-1-(4-(4-fluorophenyl)-1H-imidazol-2-yl)ethylcarbamate as a solid. LC-MS: m/z 435 (M+H)⁺. ¹H NMR (CDCl₃, 500 MHz) δ (ppm): 7.63 (1H, br), 7.61 (1H, br), 7.28 (1H, d, J=8.5 Hz), 7.21 (t, J=7 Hz), 7.07 (5H, m), 6.83 (1H, s), 5.58 (1H, br), 5.03 (1H, q, J=7.5 Hz), 3.7 (3H, s, 3.54 (1H, br), 3.41 (1H, dd, J=14.5, 7 Hz), 2.23 (1H, br), 1.41 (9H, s).

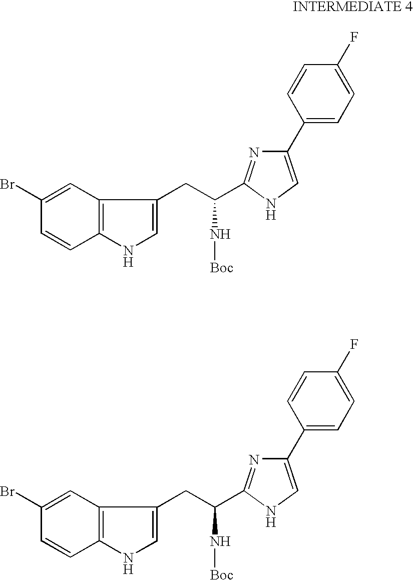

### tert-Butyl (1R)- and (1S)-2-(5-bromo-1H-indol-3-yl)-1-(4-(4-fluorophenyl)-1H-imidazol-2-yl)-1-ethylcarbamate

### Step A: N^{α}-tert-Butoxycarbonyl-5-bromo-tryptophan

A 100 mL one-neck round bottom flask was charged with D,L-5-bromo-tryptophan (2.06 g, 7.28 mmol), methanol (20 mL) and DIPEA (1.81 g, 14.55 mmol). The mixture was stirred while di-tert-butyl dicarbonate (1.91 g, 8.72 mmol) was added. The mixture was stirred for 30 min. The solvent was then removed by rotary evaporation and the residue was partitioned between ethyl acetate (30 mL) and 1N HCl (15 mL, pH=4). The organic layer was separated and the aqueous layer was extracted three times with ethyl acetate. The combined organic phases were washed with brine, dried over MgSO₄, filtered and concentrated to afford the title compound. LC-MS: m/z 383 (M+H)⁺.

### Step B: N^{α}-tert-Butoxycarbonyl-5-bromo-tryptophan, 2-(4-fluorophenyl)-2-oxoethyl ester

A 100 mL one-neck round bottom flask was charged with N^{α}-tert-butoxycarbonyl-5-bromo-tryptophan (2.78 g, 7.25 mmol), cesium carbonate (1.25 g, 3.84 mmol), and ethanol (20 mL). The mixture was stirred at rt for 30 min and the solvent was removed by rotary evaporation. To the resulting salt in DMF (20 mL) was added 2-bromo-4′-fluoroacetophenone (1.61 g, 7.40 mmol). The mixture was stirred at rt under nitrogen for 18 h. The solvent was removed by rotary evaporation and the residue was diluted with ethyl acetate (100 mL). The CsBr was filtered and washed with ethyl acetate. The filtrate was concentrated to afford N^{α}-tert-butoxycarbonyl-5-bromo-tryptophan, 2-(4-fluorophenyl)-2-oxoethyl ester as a solid. LC-MS: m/z 519 (M+

### Step C: tert-Butyl (1R,S)-2-(5-bromo-1H-indol-3-yl)-1-(4-(4-fluorophenyl)-1H-imidazol-2-yl)-1-ethylcarbamate

To a 100 mL one-neck round bottom flask was charged with N^{α}-tert-butoxycarbonyl-5-bromo-tryptophan, 2-(4-fluorophenyl)-2-oxoethyl ester (3.77 g, 7.26 mmol), ammonium acetate (2.34 g, 29 mmol) and xylene (40 mL). The mixture was then heated at reflux temperature for 3 h. After cooling to rt, the mixture was diluted with ethyl acetate (100 mL) and then washed with water, saturated aqueous NaHCO₃, brine, dried over MgSO₄, filtered and concentrated. The crude product was purified by MPLC (120 g silica gel, eluting with 0 to 40% ethyl acetate in hexanes) to afford tert-butyl (1R,S)-2-(5-bromo-1H-indol-3-yl)-1-(4-(4-fluorophenyl)-1H-imidazol-2-yl)-1-ethylcarbamate as a solid. LC-MS: m/z 599 (M+

### Step D: Resolution of the enantiomers of tert-butyl (1R,S)-2-(5-bromo-1H-indol-3-yl)-1-(4-(4-fluorophenyl)-1H-imidazol-2-yl)-1-ethylcarbamate

A solution of tert-butyl (1R,S)-2-(5-bromo-1H-indol-3-yl)-1-(4-(4-fluorophenyl)-1H-imidazol-2-yl)-1-ethylcarbamate (2.48 g, 4.97 mmol) in isopropanol (40 mL) was resolved on a ChiralCel® OD® column (2×25 cm) eluting with 12% isopropanol in heptane. The retention time of the faster-eluting enantiomer was 14.1 min, and the retention time of the slower-eluting enantiomer was 21.6 min. LC-MS: m/z 501 (M+H)⁺ (2 min).

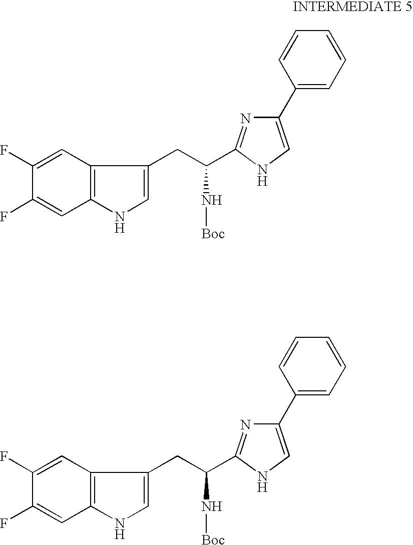

### tert-Butyl 1(R)- and 1(S)-2-(5,6-difluoro-1H-indol-3-yl)-1-(4-phenyl-1H-imidazol-2-yl)-1-ethylcarbamate

### Step A: 1-Nitro-3,4-difluoro-6-methylbenzene

To a stirred solution of 3,4-difluorotoluene (25.6 g, 0.2 mol) in H₂SO₄ (100 mL) was added KNO₃ (20.2 g, 0.2 mol) at 0° C. The resulting mixture was stirred overnight at rt. The reaction mixture was poured into ice/water (200 g) and extracted three times with EtOAc (300 mL). The combined organic layers were washed with brine (200 mL), dried and concentrated to give the title compound as a pale yellow solid. ¹H NMR (300 MHz, CDCl₃): δ 7.90˜7.96 (m, 1H), 7.13˜7.19 (m, 1H), 2.60 (s, 3H).

### Step B: 1-Diethylamino-2-(4,5-difluoro-2-nitrophenyl)-ethylene

A mixture of N,N-dimethylformamide diisopropyl acetal (11.2 g, 64 mmol) and 1-nitro-3,4-difluoro-6-methylbenzene (5 g, 32 mmol) in dry DMF was heated at 120° C. for 10 h. The resulting dark red solution was concentrated under reduced pressure and partitioned between ethyl acetate and water. The organic layer was washed with brine, dried over anhydrous sodium sulfate, filtered and concentrated under reduced pressure to give crude 1-diethylamino-2-(4,5-difluoro-2-nitrophenyl)-ethylene as a black solid which was used in the next step without further purification.

### Step C: 5,6-Difluoro-1H-indole

Zinc powder was added in portions to a solution of 1-diethylamino-2-(4,5-difluoro-2-nitrophenyl)-ethylene (17.3 g, 76 mmol) in 80% AcOH over 4 h at 75° C. The reaction mixture was cooled and filtered. The solid was dissolved in EtOAc, washed with water and brine, dried over MgSO₄, evaporated in vacuo to afford 5,6-difluoro-1H-indole which was purified by flash column chromatography on silica gel eluting with 50:1 petroleum ether/ether. ¹H NMR (300 MHz, CDCl₃): δ 8.143 (s, 1H), 7.09˜7.40 (m, 3H), 6.44˜6.51 (m, 1H).

### Step D: N^{α}-Acetyl-5,6-difluoro-tryptophan

L-Serine was dissolved in a solution of 5,6-difluoro-1H-indole (3.83 g, 25 mmol) in AcOH and Ac₂O, and the mixture was stirred at 73° C. for 2 h under N₂. After cooling, the reaction mixture was diluted with MTBE and adjusted to pH=10 with 30% aq. NaOH. Further MTBE was added to the water phase and separated. The organic layer was further extracted with 1N NaOH and a small amount of Na₂S₂O₄ was added to the combined alkali solution which was concentrated to one-half the volume, acidified with HCl to pH=3, and extracted with EtOAc. The combined organic layers were dried over anhydrous Na₂SO₄ and evaporated. The crude product was purified by flash column chromatography on silica gel (eluting with CH₂Cl₂: MeOH=15:1) to give N^{α}-acetyl-5,6-difluoro-tryptophan as a black oil. ¹H NMR (300 MHz, DMSO-d₆): δ 11.00 (s, 1H), 8.06˜8.91 (m, 1H), 7.43˜7.50 (m, 1H), 7.27˜7.33 (m, 1H), 7.18 (s, 1H), 4.36˜4.43 (m, 1H), 3.06˜3.13 (m, 1H), 2.87˜2.97 (m, 1H), 1.77 (s, 3H). LC-MS: m/z 283 (M+H)⁺.

### Step E: 5,6-Difluoro-tryptophan

A mixture of N^{α}-acetyl-5,6-difluoro-tryptophan (1.8 g, 6.38 mmol) and HCl/H₂O (10 mL/10 mL) was heated at 100° C. for 16 h. The solvent was removed under reduced pressure to afford 5,6-difluoro-tryptophan as a crude product that was used in the next step without further purification. LC-MS: m/z 241 (M+H)⁺ (6 min).

### Step F: N^{α}-tert-Butyloxycarbonyl-5,6-difluoro-tryptophan

A mixture of 5,6-difluoro-tryptophan (1.53 g, 6.38 mmol), triethylamine (2.23 mL, 15.9 mmol), and di-tert-butyl dicarbonate (1.67 g, 7.66 mmol) in dry anhydrous dichloromethane (20 mL) was stirred at rt for 1 h. The solvent was removed under reduced pressure and the residue was partitioned between ethyl acetate and water (50 mL/20 mL). The organic layer was washed with brine, dried over anhydrous magnesium sulfate, filtered and concentrated under reduced pressure to give N^{α}-tert-butyloxycarbonyl-5,6-difluoro-tryptophan which was used in the next step without further purification. LC-MS: m/z 363 (M+Na)⁺ (2 min).

### Step G: N^{α}-tert-Butyloxycarbonyl-5,6-difluoro-tryptophan, 2-(4-fluorophenyl)-2-oxoethyl ester

To a solution of N^{α}-tert-butyloxycarbonyl-5,6-difluoro-tryptophan (1.2 g, 3.53 mmol) in anhydrous DMF (15 mL) was added cesium carbonate (0.574 g, 1.76 mmol). After stirring at rt for 30 min, 2-bromoacetophenone (0.737 g, 3.7 mmol) was added to the mixture. The resulting mixture was stirred at rt for 1 h. After quenching with ethyl acetate and water (50 mL/20 mL), the aqueous layer was extracted twice with ethyl acetate (50 mL). The combined ethyl acetate layers were washed with brine, dried over anhydrous magnesium sulfate, filtered and concentrated to dryness. The residue was purified by flash column chromatography on silica gel eluting with 40% ethyl acetate in hexane to give N^{α}-tert-butyloxycarbonyl-5,6-difluoro-tryptophan, 2-(4-fluorophenyl)-2-oxoethyl ester. LC-MS: m/z 481 (M+Na)⁺ (2 min).

### Step H: tert-Butyl 1 (R,S)-2-(5,6-difluoro-1H-indol-3-yl)-1-(4-phenyl-1H-imidazol-2-yl)-1-ethylcarbamate

A mixture of N^{α}-tert-butyloxycarbonyl-5,6-difluoro-tryptophan, 2-(4-fluorophenyl)-2-oxoethyl ester (1.6 g, 3.53 mmol) and ammonium acetate (0.81 g, 10.6 mmol) in xylene (10 mL) was heated to 145° C. for 2 h. The solvent was removed under reduced pressure and the residue was partitioned between ethyl acetate and saturated NaHCO₃ solution (60 mL/40 mL). The aqueous layer was extracted twice with ethyl acetate (50 mL). The combined ethyl acetate was washed with brine, dried over anhydrous magnesium sulfate, filtered and concentrated to dryness. The residue was purified by flash column chromatography on silica gel eluting with 5% MeOH in dichloromethane to give tert-butyl 1(R,S)-2-(5,6-difluoro-1H-indol-3-yl)-1-(4-phenyl-1H-imidazol-2-yl)-1-ethylcarbamate. LC-MS: m/z 439 (M+H)⁺ (2 min).

### Step I: Resolution of enantiomers of tert-butyl 1 (RS)-2-(5,6-difluoro-1H-indol-3-yl)-1-(4-phenyl-1H-imidazol-2-yl)-1-ethylcarbamate

A solution of tert-butyl 1(R,S)-2-(5,6-difluoro-1H-indol-3-yl)-1-(4-phenyl-1H-imidazol-2-yl)-1-ethylcarbamate (0.92 g, 2.09 mmol) in isopropanol (20 mL) was resolved on an OD column eluting with 12% isopropanol in heptane. The retention time of the faster-eluting enantiomer was 13.5 min, and the retention time of the slower-eluting enantiomer was 22.5 min. Both enantiomers gave the same LC-MS: m/z 439 (M+H)⁺ (2 min).

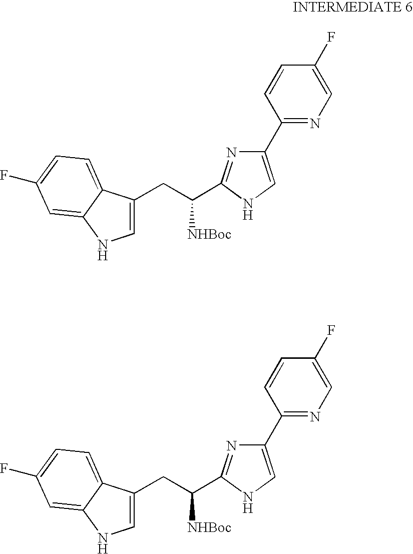

### tert-Butyl 1(R)- and 1(S)-2-(6-fluoro-1H-indol-3-yl)-1-(4-(4-fluoropyridin-2-yl)-1H-imidazol-2-yl)-1-ethylcarbamate

### Step A: Ethyl 5-fluoropyridine-2-carboxylate

To a stirred solution of 2-bromo-5-fluoropyridine (5 g, 28.4 mmol), dry ethanol (20 mL, 343 mmol), triethylamine (7.92 mL, 56.8 mmol), triphenylphosphine (2.98 g, 11.36 mmol) and palladium acetate (1.276 g, 5.68 mmol) in DMF was purged with carbon monoxide (CO) gas for about 30 min. The reaction flask was then equipped with a CO balloon and the mixture was stirred at 60° C. for 5 d in the presence of CO. After cooling to rt, the reaction mixture was poured onto cold water (100 mL), and the product was extracted three times with ether (150 mL). The combined organic extracts were dried over anhydrous sodium sulfate, filtered and concentrated in vacuo. The residue was purified by MPLC eluting with 0% EtOAc-20% EtOAc in hexane to give ethyl 5-fluoropyridine-2-carboxylate. ¹H NMR (500 MHz, CDCl₃): δ 8.64-8.62 (m, 1H), 8.24-8.20 (m, 1H), 7.58-7.54 (m, 1H), 4.52-4.50 (m, 2H), 1.50-1.45 (m, 3H). LC-MS found for C₈H₈FNO₂: m/z 170.07 (M+H)⁺.

### Step B: 5-Fluoropyridine-2-carboxylic acid

To a stirred solution of ethyl 5-fluoropyridine-2-carboxylate (3.5 g, 20.69 mmol) in THF (20 mL) was added lithium hydroxide monohydrate (4.34 g, 103 mmol) in water (20 mL). The mixture was stirred at rt overnight, the pH adjusted to about 7 using 1N HCl in water, and evaporated to dryness to give 5-fluoropyridine-2-carboxylic acid along with lithium chloride. LC-MS found for C₆H₄FNO₂: m/z 142.15 (M+H)⁺ (0.6 min).

### Step C: 2-Bromo-1-(5-fluoropyridin-2-yl)ethanone

To a stirred suspension of 5-fluoropyridine-2-carboxylic acid (2.95 mmol with consideration of lithium chloride contamination) in methylene chloride (20 mL) at it was added oxalyl chloride (2.0 M in DCM, 4.43 mL, 8.85 mmol) dropwise, followed by addition of DMF (0.1 mL). The mixture was stirred at it for 30 min, the solid was then filtered off and washed with DCM. The filtrate was concentrated to one-third of the original volume and anhydrous THF (20 mL) was added. To this solution was added trimethylsilyldiazomethane (2.0 in ether, 5.90 mL, 11.80 mmol) dropwise at 0° C. The mixture was stirred at it for an additional 30 min, then cooled to 0° C. again, followed by dropwise addition of concentrated HBr (48% in water, 1 mL, 8.85 mmol). After bubbling ceased, the mixture was allowed to stir at it for 30 min and was then concentrated in vacuo to give crude 2-bromo-1-(5-fluoropyridin-2-yl)ethanone which was used in the subsequent reaction. LC-MS found for C₇H₅BrFNO: m/z 218.02 (M+H)⁺ (2.18 min).

### Step D: N^{α}-tert-Butyloxycarbonyl-6-fluoro-tryptophan

To the stirred suspention of 6-fluoro-D,L-tryptophan (5.82 g, 26.0 mmol) in dioxane (80 mL) was added 1N NaOH (30 mL) and di-tert-butyl dicarbonate (6.286 g, 2.85 mmol). The mixture was stirred at it overnight, and the pH adjusted to about 6-7 with 1N HCl. The product was extracted three times with EtOAc. The combined organic extracts were dried over anhydrous sodium sulfate, filtered and evaporated to dryness to give the title compound. LC-MS found for C₆H₁₉FN₂O₄: m/z 345.2 (M+Na)⁺ (2.83 min).

### Step E: N^{α}-tert-Butyloxycarbonyl-6-fluoro-tryptophan, 2-(5-fluoropyridin-2-yl)-2-oxoethyl ester

To a stirred solution of N^{α}-tert-butyloxycarbonyl-6-fluoro-tryptophan (3.0 g, 9.31 mmol) in anhydrous ethanol (21 mL) was added cesium carbonate (3.03 g, 9.31 mmol). The suspension was stirred at it for 30 min and then evaporated to dryness followed by addition of dry DMF (36 mL). To this stirred suspension was added 2-bromo-1-(5-fluoropyridin-2-yl)ethanone (3.34 g, 11.17 mmol). The mixture was stirred at it overnight and then evaporated. To the residue was added EtOAc, and then the solid was filtered off and washed with EtOAc. The combined filtrates were concentrated and the crude product purified by MPLC using 50% EtOAc in hexane as the eluting solvent to give N^{α}-tert-butyloxycarbonyl-6-fluoro-tryptophan, 2-(5-fluoropyridin-2-yl)-2-oxoethyl ester. LC-MS found for C₂₃H₂₃F₂N₃O₅: m/z 482.26 (M+Na)⁺ (1.19 min).

### Step F: tert-Butyl 1(R,S)-2-(6-fluoro-1H-indol-3-yl)-1-(4-(4-fluoropyridin-2-yl)-1H-imidazol-2-yl)-1-ethylcarbamate

A mixture of N^{α}-tert-butyloxycarbonyl-6-fluoro-tryptophan, 2-(5-fluoropyridin-2-yl)-2-oxoethyl ester (583 mg, 1.26 mmol) and ammonium acetate (978 mg, 12.69 mmol) in anhydrous xylene (30 mL) was heated at reflux temperature for 4 h. After cooling to rt, the reaction mixture was concentrated, and the residue was partitioned between EtOAc (50 mL) and saturated aq. NaHCO₃ (50 mL). The product was extracted three times with EtOAc (50 mL), and the combined organic extracts were combined, dried over sodium sulfate and evaporated. The crude product was purified by MPLC using EtOAc as the eluting solvent to give tert-butyl 1(R,S)-2-(6-fluoro-1H-indol-3-yl)-1-(4-(4-fluoropyridin-2-yl)-1H-imidazol-2-yl)-1-ethylcarbamate. ¹H NMR (500 MHz, CDCl₃): δ 8.41-8.39 (1H), 8.0-7.9 (1H), 7.7-7.4 (3H), 7.1-6.9 (2H), 6.8-6.7 (1H), 5.18-4.95 (1H), 3.20-3.40 (2H), 1.40-30 (9H). LC-MS found for C₂₃H₂₃F₂N₅O₂: m/z 440.14 (M+H)⁺ (1.03 min).

### Step G: Resolution of the enantiomers of ten-butyl 1(R,S)-2-(6-fluoro-1H-indol-3-yl)-1-(4-(4-fluoropyridin-2-yl)-1H-imidazol-2-yl)-1-ethylcarbamate

tert-Butyl 1(R,S)-2-(6-fluoro-1H-indol-3-yl)-1-(4-(4-fluoropyridin-2-yl)-1H-imidazol-2-yl)-1-ethylcarbamate (650 mg) was resolved on a Gilson system using ChiralCel® OD column (2 cm×25 cm), 15% IPA in heptane as mobile phase, flow rate of 9 mL/min, wavelength of 220 nm, and about 50 mg per run and run time of 60 min to give each individual enantiomer of tert-butyl 2-(6-fluoro-1H-indol-3-yl)-1-(4-(4-fluoropyridin-2-yl)-1H-imidazol-2-yl)-1-ethylcarbamate. LC-MS found for both isomers with C₂₃H₂₃F₂N₅O₂: m/z 440.14 (M+H)⁺ (1.03 min).

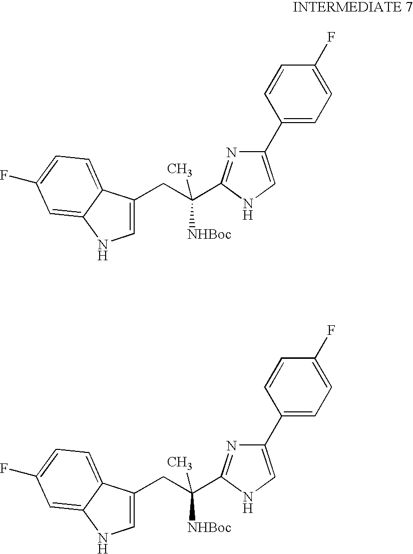

### tert-Butyl 1(R)- and 1(S)-2-(6-fluoro-1H-indol-3-yl)-1-(4-(4-fluoropyridin-2-yl)-1H-imidazol-2-yl)-1-methyl-1-ethylcarbamate

### Step A: 1-(6-Fluoro-1H-indol-3-yl)-N,N-dimethylmethanamine

A 500 mL one-neck round bottom flask was charged with 6-fluoroindole (5 g, 37.0 mmol), dimethylamine hydrochloride (9.05 g, 111 mmol), paraformaldehyde (1.33 g, 44.4 mmol) and 1-butanol (100 mL). The resulting mixture was stirred and heated at reflux temperature for 1 h. After cooling to rt, the mixture was diluted with ethyl acetate (100 mL) and washed with 1N NaOH (120 mL). The organic layer was separated and the aqueous layer was extracted three times with ethyl acetate (100 mL). The combined organic phases were washed with water, brine, dried over MgSO₄, filtered and concentrated to afford 1-(6-fluoro-1H-indol-3-yl)-N,N-dimethylmethanamine as a light-colored solid. LC-MS: m/z 193 (M+H)⁺.

### Step B: Ethyl 3-(6-fluoro-1H-indol-3-yl)-2-methyl-2-nitropropanoate

A 100 mL three-neck round bottom flask was charged with 1-(6-fluoro-1H-indol-3-yl)-N,N-dimethylmethanamine (7.11 g, 37.0 mmol), ethyl 2-nitropropionate (5.99 g, 40.7 mmol), and xylene (100 mL). The flask was equipped with a condenser, a nitrogen inlet and septum. The mixture was stirred and heated at reflux temperature with a steady nitrogen flow for 8 h. The mixture was then concentrated by rotary evaporation and the residue was purified by MPLC (120 g silica gel, eluting with 0 to 30% ethyl acetate in hexanes) to afford ethyl 3-(6-fluoro-1H-indol-3-yl)-2-methyl-2-nitropropanoate. LC-MS: m/z 295 (M+H)⁺ (3.23 min). ¹H NMR (CDCl₃, 500 MHz) δ (ppm): 8.15 (1H, s), 7.45 (1H, dd, J=8.5, 5 Hz), 7.04 (1H, dd, J=9.5, 2 Hz), 6.99 (1H, d, J=2 Hz), 6.91 (1H, td, J=5, 2 Hz), 4.27 (2H, m), 3.78 (1H, d, J=15 Hz), 3.60 (1H, d, J=15 Hz), 1.73 (3H, s), 1.27 (3H, m).

### Step C: 6-Fluoro-α-methyltryptophan, ethyl ester

To a 500 mL one-neck round bottom flask was charged with ethyl 3-(6-fluoro-1H-indol-3-yl)-2-methyl-2-nitropropanoate (7.02 g, 23.85 mmol), zinc (9.36 g, 143 mmol) and acetic acid (100 mL). The mixture was then stirred and heated at 70° C. for 1 h. After cooling to rt, the solid was removed by filtration and washed with ethyl acetate. The filtrate was concentrated by rotary evaporation and the residue was then partitioned between ethyl acetate (100 mL) and saturated aqueous sodium hydrogencarbonate solution (100 mL). The organic layer was separated and the aqueous layer was extracted three times with ethyl acetate. The combined organic phases were washed with brine, dried over magnesium sulfate, filtered and concentrated to afford 6-fluoro-α-methyltryptophan, ethyl ester as a white solid. LC-MS: m/z 265 (M+H)⁺ (0.90 min).

### Step D: N^{α}-tert-Butyloxycarbonyl-6-fluoro-α-methyltryptophan, ethyl ester

To a 250 mL one-neck round bottom flask was charged with 6-fluoro-α-methyltryptophan, ethyl ester (5.76 g, 21.79 mmol), THF (100 mL) and triethylamine (6.62 g, 65.4 mmol). The mixture was stirred while di-tert-butyl dicarbonate (7.13 g, 32.7 mmol) was added in one portion and the reaction mixture was stirred for 20 h. The reaction was then quenched with water (30 mL). The organic layer was separated and the aqueous layer was extracted twice with ethyl acetate. The combined organic phases were washed with water, brine, dried over MgSO₄, filtered and concentrated. The residue was purified by MPLC (120 g silica gel, eluting with 10 to 100% ethyl acetate in hexanes) to afford N^{α}-tert-butyloxycarbonyl-6-fluoro-α-methyltryptophan, ethyl ester. LC-MS: m/z 365 (M+H)⁺ (1.18 min). ¹H NMR (CDCl₃, 500 MHz) δ (ppm): 8.08 (1H, s), 7.49 (1H, dd, J=9, 5.5 Hz), 7.01 (1H, dd, 6.95 (1H, s), 6.86 (1H, td), 5.18 (1H, br), 4.22 (2H, m), 3.46 (1H, br), 3.35 (1H, d, J=14.5 Hz), 1.56 (3H, s), 1.44 (9H, s), 1.24 (3H, m).

### Step E: N^{α}-tert-Butyloxycarbonyl-6-fluoro-α-methyltryptophan

A 250 mL one-neck round bottom flask was charged with N^{α}-tert-butyloxycarbonyl-6-fluoro-α-methyltryptophan, ethyl ester (5.29 g, 14.52 mmol) and methanol (40 mL). The mixture was stirred while a solution of 5N NaOH (20 mL) was added and the resulting reaction mixture was heated at 60° C. for 1 h. The mixture was concentrated to one-third the volume and then partitioned between water (10 mL) and ethyl acetate (40 mL). The pH of the aqueous layer was adjusted to 2 with concentrated HCl (about 6 mL). The organic layer was separated and the aqueous layer was extracted twice with ethyl acetate (40 mL). The combined organic phases were washed with water, dried over MgSO₄, filtered and concentrated to afford N^{α}-tert-butyloxycarbonyl-6-fluoro-α-methyltryptophan. LC-MS: m/z 337 (M+H)⁺.

### Step F: N^{α}-tert-Butyloxycarbonyl-6-fluoro-α-methyltryptophan, 2-(4-fluorophenyl)-2-oxoethyl ester

A 250 mL one-neck round bottom flask was charged with N^{α}-tert-butyloxycarbonyl-6-fluoro-α-methyltryptophan (4.88 g, 14.51 mmol), cesium carbonate (4.73 g, 14.51 mmol) and DMF (40 mL). The mixture was stirred while 2-bromo-4′-fluoroacetophenone (3.46 g, 15.96 mmol) was added. The mixture was stirred at rt under nitrogen for 2 h. The solvent was removed by rotary evaporation and the residue was diluted with ethyl acetate (100 mL). The CsBr₂ solid was filtered and washed with ethyl acetate. The filtrate was concentrated to afford N^{α}-tent-butyloxycarbonyl-6-fluoro-α-methyltryptophan, 2-(4-fluorophenyl)-2-oxoethyl ester. LC-MS: m/z 473 (M+H)⁺.

### Step G: tert-butyl (1R,S)-2-(6-fluoro-1H-indol-3-yl)-1-[4-(4-fluorophenyl)-1H-imidazol-2-yl]-1-methyl-1-ethylcarbamate

A 500 mL one-neck round bottom flask was charged with 1\1″-tert-butyloxycarbonyl-6-fluoro-α-methyltryptophan, 2-(4-fluorophenyl)-2-oxoethyl ester (6.85 g, 14.51 mmol), ammonium acetate (6.71 g, 87 mmol) and xylene (40 mL). The mixture was then heated to reflux for 3 h. After cooling to rt, the mixture was diluted with ethyl acetate (200 mL) and then washed with saturated aqueous sodium hydrogencarbonate solution, water, brine, dried over MgSO₄, filtered and concentrated. The crude product was purified by MPLC (120 g silica gel, eluting with 10 to 60% ethyl acetate in hexanes) to afford tert-butyl (1R,S)-2-(6-fluoro-1H-indol-3-yl)-1-[4-(4-fluorophenyl)-1H-imidazol-2-yl]-1-methyl-1-ethylcarbamate. LC-MS: m/z 453 (M+H)⁺. ¹H NMR (CDCl₃, 500 MHz) δ (ppm): 7.65 (2H, br), 7.17 (2H, m), 7.08 (2H, t, J=8.5 Hz), 6.96 (1H, dd), 7.79 (1H, s), 6.64 (1H, t), 3.44 (2H, br), 1.65 (3H, s), 1.42 (9H, br).

### Step H: Resolution of the enantiomers of tert-butyl (1R,S)-2-(6-fluoro-1H-indol-3-yl)-1-[4-(4-fluorophenyl)-1H-imidazol-2-yl]-1-methyl-1-ethylcarbamate

tert-Butyl (1R,S)-2-(6-fluoro-1H-indol-3-yl)-1-[4-(4-fluorophenyl)-1H-imidazol-2-yl]-1-methyl-1-ethylcarbamate was resolved on a ChiralPak® AD® column eluting with 20% IPA/heptane to provide each individual enantiomer: (Rₜ=10.4 min on chiral AD by 20% IPA in heptane) and (Rₜ=17.2 min on chiral AD column by 20% IPA in heptane).

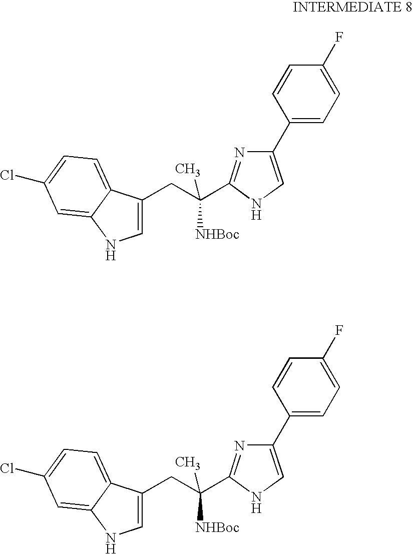

### tert-Butyl (1R)- and (1S)-2-(6-chloro-1H-indol-3-yl)-1-(4-(4-fluorophenyl)-1H-imidazol-2-yl)-1-ethylcarbamate

### Step A: 1-(6-Chloro-1H-indol-3-yl)-N,N-dimethylmethanamine

A 500 mL one-neck round bottom flask was charged with 6-chloroindole (17.39 g, 115 mmol), dimethylamine hydrochloride (28.1 g, 344 mmol), paraformaldehyde (4.13 g, 138 mmol) and 1-butanol (200 mL). The resulting reaction mixture was then stirred and heated at reflux temperature for 1 h. After cooling to rt, the mixture was diluted with ethyl acetate (100 mL) and washed with 1N NaOH (120 mL). The organic layer was separated and the aqueous layer was extracted three times with ethyl acetate (100 mL). The combined organic phases were washed with water, brine, dried over MgSO₄, filtered and concentrated to afford 1-(6-chloro-1H-indol-3-yl)-N,N-dimethylmethanamine as a light-colored solid. LC-MS: m/z 209 (M+H)⁺.

### Step B: Ethyl 3-(6-chloro-1H-indol-3-yl)-2-methyl-2-nitropropanoate

A 1000 mL three-neck round bottom flask was charged with 1-(6-chloro-1H-indol-3-yl)-N,N-dimethylmethanamine (23.95 g, 115 mmol), ethyl 2-nitropropionate (18.57 g, 126 mmol), and xylene (200 mL). The flask was equipped with a condenser, a nitrogen inlet and septum. The mixture was stirred and heated to reflux with a steady nitrogen flow for 8 h. The mixture was then concentrated by rotary evaporation and the residue was purified by MPLC (330 g silica gel, eluting with 0 to 30% ethyl acetate in hexanes) to afford ethyl 3-(6-fluoro-1H-indol-3-yl)-2-methyl-2-nitropropanoate as a sticky oil. LC-MS: m/z 311 (M+H)⁺. NMR (CDCl₃, 500 MHz) δ (ppm): 8.16 (1H, s), 7.45 (1H, dd), 7.35 (1H, d), 7.06 (1H, dd), 7.00 (1H, d), 4.27 (2H, m), 3.78 (1H, d, J=15 Hz), 3.60 (1H, d, J=15 Hz), 1.73 (3H, s), 1.27 (3H, m).

### Step C: 6-Chloro-α-methyltryptophan, ethyl ester

A 500 mL one-neck round bottom flask was charged with ethyl 3-(6-chloro-1H-indol-3-yl)-2-methyl-2-nitropropanoate (26.3 g, 85 mmol), zinc (33.2 g, 508 mmol) and acetic acid (200 mL). The mixture was then stirred and heated at 70° C. for 1 h. After cooling to rt, the solid was removed by filtration and washed with ethyl acetate. The filtrate was concentrated by rotary evaporation and the residue was then partitioned between ethyl acetate (200 mL) and saturated aqueous sodium hydrogencarbonate solution (200 mL). The organic layer was separated and the aqueous layer was extracted three times with ethyl acetate. The combined organic phases were washed with brine, dried over magnesium sulfate, filtered and concentrated to afford 6-chloro-α-methyltryptophan, ethyl ester as a white solid. LC-MS: m/z 281 (M+H)⁺ (1.20 min).

### Step D: N^{α}-tert-Butoxycarbonyl-6-chloro-α-methyltryptophan, ethyl ester

To a 250 mL one-neck round bottom flask was charged with 6-chloro-α-methyltryptophan, ethyl ester (23.76 g, 85 mmol), THF (300 mL) and triethylamine (25.7 g, 254 mmol). The mixture was stirred while di-tert-butyl dicarbonate (27.7 g, 127 mmol) was added in one portion and the reaction mixture was stirred for 20 h. The reaction mixture was concentrated and the residue was purified by MPLC (330 g silica gel, eluting with 10 to 100% ethyl acetate in hexanes) to afford N^{α}-tert-butoxycarbonyl-6-chloro-α-methyltryptophan, ethyl ester. LC-MS: m/z 381 (M+H)⁺ (1.18 min). ¹H NMR (CDCl₃, 500 MHz) δ (ppm): 8.45 (1H, s), 7.46 (1H, dd, J=9 Hz), 7.29 (1H, s) 7.03 (1H, d), 6.92 (1H, s), 5.20 (1H, br), 4.22 (2H, m), 3.40 (1H, br), 3.35 (1H, d, J=14 Hz), 1.59 (3H, s), 1.44 (9H, s), 1.24 (3H, m).

### Step E: N^{α}-tert-Butoxycarbonyl-6-chloro-α-methyltryptophan

A mixture of N^{α}-tent-butoxycarbonyl-6-chloro-α-methyltryptophan, ethyl ester (4.28 g, 11.24 mmol), sodium hydroxide (2.7 g, 67.4 mmol) and MeOH/H₂O (38 mL/19 mL) was heated at 55° C. for 4 h. The solvent was removed under reduced pressure and the residue was partitioned between ethyl acetate and H₂O (50 mL/50 mL). The pH was adjusted to about 6 with concentrated HCl, and the aqueous layer was extracted twice with ethyl acetate (100 mL). The combined extracts was washed with brine, dried over anhydrous magnesium sulfate, filtered and concentrated to dryness to afford N^{α}-tert-butoxycarbonyl-6-chloro-α-methyltryptophan which was used to the next step without further purification. LC-MS: m/z 352 (M+H)⁺ (2 min).

### Step F: N^{α}-tert-Butoxycarbonyl-6-chloro-α-methyltryptophan, 2-(4-fluorophenyl)-2-oxoethyl ester

A mixture of N^{α}-tert-butoxycarbonyl-6-chloro-α-methyltryptophan (3.8 g, 10.77 mmol) in anhydrous DMF (30 mL) was added cesium carbonate (3.5 g, 10.7 mmol). After stirring at rt for 30 min, 2-bromo-4-fluoroacetophenone (2.45 g, 11.3 mmol) was added to the mixture. The resulting mixture was stirred at rt for 16 h. The reaction was quenched with ethyl acetate and water (100 mL/50 mL). The aqueous layer was extracted twice with ethyl acetate (100 mL). The combined ethyl acetate extracts were washed with brine, dried over anhydrous magnesium sulfate, filtered and concentrated to dryness. The residue was purified by flash column chromatography on silica gel eluting with 20% ethyl acetate in hexane to give N^{α}-tert-butoxycarbonyl-6-chloro-α-methyltryptophan, 2-(4-fluorophenyl)-2-oxoethyl ester. LC-MS: m/z 489 (M+H)⁺ (2 min).

### Step G: tert-Butyl (1R,S)-2-(6-chloro-1H-indol-3-yl)-1-(4-(4-fluorophenyl)-1H-imidazol-2-yl)-1-methyl-1-ethylcarbamate

A mixture of N^{α}-tert-butoxycarbonyl-6-chloro-α-methyltryptophan, 2-(4-fluorophenyl)-2-oxoethyl ester (3.35 g, 6.85 mmol) and ammonium acetate (2.11 g, 27.4 mmol) in xylene (20 mL) was heated at 145° C. for 2 h. The solvent was removed under reduced pressure and the residue was partitioned between ethyl acetate and saturated aq. NaHCO₃ solution (100 mL/50 mL). The aqueous layer was extracted twice with ethyl acetate (100 mL). The combined ethyl acetate extracts were washed with brine, dried over anhydrous magnesium sulfate, filtered and concentrated to dryness. The residue was purified by flash column chromatography eluting with 60% ethyl acetate in hexane to give the title compound. LC-MS: m/z 469 (M+H)⁺ (2 min).

### Step H: Resolution of the enantiomers of tert-butyl (1R,S)-2-(6-chloro-1H-indol-3-yl)-1-(4-(4-fluorophenyl)-1H-imidazol-2-yl)-1-methyl-1-ethylcarbamate

A solution of tert-butyl (1R,S)-2-(6-chloro-1H-indol-3-yl)-1-(4-(4-fluorophenyl)-1H-imidazol-2-yl)-1-methyl-1-ethylcarbamate (1.0 g, 2.13 mmol) in isopropanol (20 mL) was resolved using a ChiralPak AD® column with 15% isopropanol in heptane as the mobile phase. The retention time of the faster-eluting enantiomer was 23.6 min, and the retention time of the slower-eluting enantiomer was 33.6 min. LC-MS: m/z 469 (M+H)⁺ (2 min).

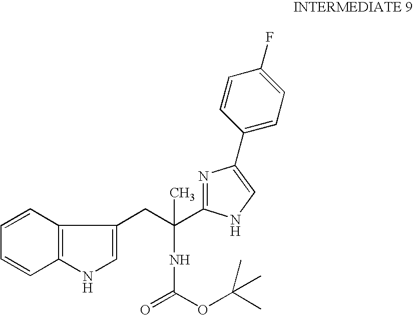

### tert-Butyl (1R,S)-2-(1H-indol-3-yl)-1-(4-(4-fluorophenyl)-1H-imidazol-2-yl)-1-methyl-1-ethylcarbamate

### Step A: tert-Butyl (1R,S)-2-(1H-indol-3-yl)-1-(4-(4-fluorophenyl)-1H-imidazol-2-yl)-1-methyl-1-ethylcarbamate

The title compound was prepared from N-Boc-α-methyl-tryptophan and 2-bromo-4′-fluoro-acetophenone by methods described in the literature (Gordon, T. et al., Bioorg. Me Chem. Lett. 1993, 3, 915; Gordon, T. et al., Tetrahedron Lett. 1993, 34, 1901; Poitout, L. et al., J. Med. Chem. 2001, 44, 2990).

### Step B: Resolution of the enantiomers of tert-butyl (1R,S)-2-(1H-indol-3-yl)-1-(4-(4-fluorophenyl)-1H-imidazol-2-yl)-1-methyl-1-ethylcarbamate

Chiral HPLC resolution of tert-butyl (1R,S)-2-(1H-indol-3-yl)-1-(4-(4-fluorophenyl)-1H-imidazol-2-yl)-1-methyl-1-ethylcarbamate (500 mg, 1.15 mmol) was carried out with a ChiralPak AD® 4.6×250 mm column, flow rate at 0.5 mL/min of 20% isopropanol in heptane, and IJV detection at 254 nm. The retention times of the faster-eluting enantiomer and the slower-eluting enantiomer were 16.2 min and 24.7 min, respectively. ¹H NMR of the faster-eluting enantiomer (500 MHz, CD₃OD): δ 7.61 (m, 2H), 7.31 (m, 2H), 7.20 (m, 1H), 7.14 (t, 2H), 7.04 (t, 1H), 6.90 (m, 2H), 3.46 (m, 2H), 1.73 (s, 3H), 1.44 (s, 9H). LC-MS: m/z 435.08 (M+H)⁺ (2.67 min). ¹H NMR and LC-MS of the slower-eluting enantiomer were identical to those of the faster-moving enantiomer.

The Intermediates shown in Table 1 were prepared from the appropriately substituted D- or D,L-tryptophan derivative and a halomethyl aryl ketone according to the methods described in the references cited in Intermediate 1 or the other Intermediates.

TABLE 1

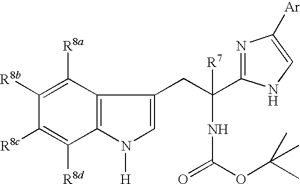

| | | | | | | | LC-MS: |
|---|---|---|---|---|---|---|---|
| | | | | | | | m/z (M + 1) |
| Intermediate | R^{8a} | R^{8b} | R^{8c} | R^{8d} | R⁷ | Ar | (ret time: min) |
| 10 | H | H | H | H | H | 4-F-Ph | 421.2 (2.75) |
| 11 | H | H | H | H | CH₃ | Ph | 417.3 (2.66) |
| 12 | F | H | H | H | H | Ph | 421.3 (1.02) |
| 13 | H | F | H | H | CH₃ | 4-F-Ph | 453.1 (1.06) |
| 14 | H | H | F | H | H | 4-F-Ph | 439.2 (2.75) |
| 15 | H | H | Br | H | H | 4-F-Ph | 499.3 (1.10) |
| 16 | H | H | H | F | CH₃ | 4-F-Ph | 453.1 (1.06) |

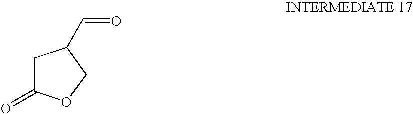

### Tetrahydrofuran-2-one-4-carboxaldehyde

### Step A: 4-Hydroxymethyl-tetrahydrofuran-2-one

The title compound was prepared from tetrahydrofuran-2-one-4-carboxylic acid according to the methods described in the literature (Mori et al., Tetrahedron. 38:2919-2911, 1982). ¹H NMR (500 MHz, CDCl₃): δ 5.02 (s, 1H), 4.42 (dd, 1H), 4.23 (dd, 1H), 3.67 (m, 2H), 2.78 (m, 1H), 2.62, (dd, 1H), 2.40, (dd, 1H).

### Step B: Tetrahydrofuran-2-one-4-carboxaldehyde

To a solution of 4-hydroxymethyl-tetrahydrofuran-2-one (200 mg, 1.722 mmol) in CH₂Cl₂ (15 mL) was added Dess-Martin periodinane (804 mg, 1.895 mmol). The reaction was stirred at rt for 2.5 h. Sodium bicarbonate (1447 mg, 17.22 mmol) and water (2 mL) were added to the reaction. After stirring for 15 min, sodium thiosulfate (2723 mg, 17.22 mmol) was added, and the suspension was stirred for 15 additional min. The suspension was dried over sodium sulfate and filtered. The solid was washed with CH₂Cl₂. The organic filtrate was concentrated to a minimal volume. ¹H NMR (500 MHz, CDCl₃) showed an aldehyde singlet at δ 9.74 ppm. The crude product was used without further purification in subsequent reactions.

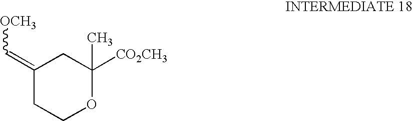

### 4-(Methoxymethylene)-2-methyl-tetrahydro-2H-pyran-2-carboxylic acid, methyl ester

### Step A: 2-Methyl-2,3-dihydro-4H-pyran-4-one-2-carboxylic acid, methyl ester

A 100 mL one-neck round bottom flask was charged with Danishefsky's diene (5 g, 29.0 mmol) along with methyl pyvurate (3.11 g, 30.5 mmol) and toluene (50 mL). The mixture was stirred while a solution of ZnCl₂ (1M solution in ether) (2.90 mL, 2.90 mmol) was added dropwise in 5 min. The resulting reaction mixture was then stirred at rt for 18 h. The reaction was quenched by adding 0.1 N HCl (50 mL) and stirred at rt for 1 h. The organic layer was separated and the aqueous layer was extracted three times with ethyl acetate. The combined organic phases were washed with water, brine, dried over sodium sulfate, filtered and concentrated. The residue was purified by MPLC (120 g silica gel, 5 to 50% ethyl acetate in hexanes as the mobile phase) to afford the product as a clear liquid. ¹H NMR (500 MHz, CDCl₃): δ 7.40 (d, 1H), 5.48 (d, 1H), 3.82 (s, 3H), 3.05 (d, 1H), 2.73 (d, 1H), 1.71, (s, 3H).

### Step B: 2-Methyl-tetrahydropyran-4-one-2-carboxylic acid, methyl ester

A suspension of 2-methyl-2,3-dihydro-4H-pyran-4-one-2-carboxylic acid, methyl este from Step A (3.54 g, 20.80 mmol) and Pd—C (2.214 g, 2.080 mmol) in methanol (50 mL) was attached to a H₂ balloon. The suspension was stirred at RT for 4 h. The reaction was filtered to remove the catalyst. The catalyst was washed was MeOH and filtrate concentrated to yield 2-methyl-tetrahydropyran-4-one-2-carboxylic acid, methyl ester. ¹H NMR (500 MHz, CDCl₃): δ 4.20 (m, 1H), 3.93 (m, 1H), 3.80 (s, 3H), 2.95 (d, 1H), 2.58 (m, 1H), 2.43 (m, 2H), 1.56 (s, 3H).

### Step C: 4-(Methoxymethylene)-2-methyl-tetrahydro-2H-pyran-2-carboxylic acid, methyl ester

A suspension of (methoxymethyl)triphenylphosphonium chloride (7.71 g, 22.51 mmol) in THF (25 mL) was cooled to −20° C. and potassium tert-butoxide (18.00 mL, 18.00 mmol) in THF was added dropwise. After 10 min, a solution of 2-methyl-tetrahydropyran-4-one-2-carboxylic acid, methyl ester from Step B (1.55 g, 9.00 mmol) in THY (15 mL) was added. The mixture was stirred for 30 min, then warmed to RT and stirred for an additional h. The mixture was cooled to −78° C. and quenched with saturated aqueous ammonium chloride. The mixture was extracted with EtOAc. The combined organic layers were washed with brine and dried over sodium sulfate. Silica gel column chromatography (hexane gradient to EtOAc) afforded 4-(methoxymethylene)-2-methyl-tetrahydro-2H-pyran-2-carboxylic acid, methyl ester as a 1:1 mixture of geometric isomers. Characteristic peaks in ¹H NMR (500 MHz, CDCl₃) are δ5.93 (s, 1H) for one isomer and 5.90 (s, 1H) for the other isomer.

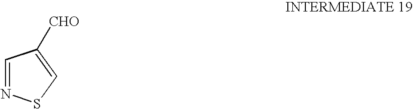

### Isothiazole-4-carboxaldehyde

### Step A: N-Methoxy-N-methyl-isothiazole-4-carboxamide

A solution of isothiazole-4-carboxylic acid (1 g, 7.74 mmol) in CH₂Cl₂ (15 mL) and DMF (0.060 mL, 0.774 mmol) was cooled to 0° C. and oxalyl chloride (0.813 mL, 9.29 mmol) was added dropwise over 10 min. The reaction mixture was warmed to RT and stirred for 1 h. The resulting acid chloride solution was added to a cooled solution of N-methoxy-N-methyl-amine hydrochloride and K₂CO₃ (4.82 g, 34.8 mmol) in water (10 mL). The mixture was stirred at RT overnight and then extracted twice with EtOAc. The combined organic layers were washed with brine, dried over anhydrous Na₂SO₄, filtered, and concentrated to yield N-methoxy-N-methyl-isothiazole-4-carboxamide. ¹H NMR (400 MHz, CDCl₃): δ 9.25 (s, 1H), 8.93 (s, 1H), 3.66 (s, 3H), 3.36 (s, 3H).

### Step B: Isothiazole-4-carboxaldehyde

Crude N-methoxy-N-methyl-isothiazole-4-carboxamide from Step A (0.91 g, 5.28 mmol) was dissolved in CH₂Cl₂ (15 mL) and cooled to −78° C. The solution was treated with DIBAL (15.85 mL, 15.85 mmol) and kept at −78° C. for 3 h. The reaction was quenched by dropwise addition of sat. aq. NH₄Cl (3 mL) at −78° C., warmed to RT and then kept cold overnight. The mixture was diluted with water and ether and treated with Rochelle's salt (6 g) and stirred at RT for 2 h. The organic layer was separated and the aqueous layer was extracted with ether. The combined organic layers were washed with brine, dried over anhydrous Na₂SO₄, and evaporated to afford isothiazole-4-carboxaldehyde which was used without further purification. ¹H NMR (500 MHz, CDCl₃): δ 10.16 (s, 1H), 9.38 (s, 1H), 9.01 (s, 1H).

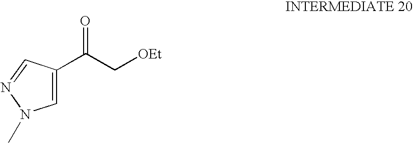

### 2-Ethoxy-1-(1-methyl-pyrazol-4-yl)-ethanone

### Step A: N-Methoxy-N-methyl-2-ethoxyacetamide

A solution of ethoxyacetic acid (4.54 mL, 48.0 mmol) in CH₂Cl₂ (80 mL) and DMF (0.372 mL, 4.80 mmol) was cooled to 0° C. and oxalyl chloride (5.05 mL, 57.6 mmol) was added dropwise over 10 min. The reaction mixture was warmed up to RT and stirred for 1 h. The resulting acid chloride solution was added to a cooled solution of N-methoxy-N-methyl-amine hydrochloride and K₂CO₃ (29.9 g, 216 mmol) in water (40 mL). The mixture was stirred at RT overnight and extracted twice with ethyl acetate. The combined organic layers were washed with brine, dried over anhydrous sodium sulfate, filtered and concentrated to afford crude N-methoxy-N-methyl-ethoxyacetamide which was purified by silica gel column chromatography eluting with a CH₂Cl₂-to-acetone gradient. NMR (500 MHz, CDCl₃): δ 4.29 (s, 2H), 3.72 (s, 3H), 3.65 (q, 2H), 3.22 (s, 3H), 1.29 (t, 3H).

### Step B: 2-Ethoxy-1-(1-methyl-pyrazol-4-yl)-ethanone

To a solution of 1-methyl-4-iodo-1H-pyrazole (3 g, 14.42 mmol) in THF (40 mL) was added isopropylmagnesium chloride (2.0M in THF) (8.00 mL, 16.01 mmol) at 0° C. The mixture was stirred at 0° C. for 1 h, cooled to −78° C., and N-methoxy-N-methyl-2-ethoxyacetamide from Step A (3.18 g, 21.63 mmol) was added. The mixture was slowly warmed to RT in 1.5 h. The reaction was cooled to −78° C. and quenched by dropwise addition of sat. aq. NH₄Cl, warmed to RT and stored in the cold overnight. The reaction was diluted with cold 1N HCl and extracted four times with EtOAc. The combined organic extracts were washed with brine, dried (Na₂SO₄) and concentrated. Silica gel chromatography eluting with a gradient of 50% EtOAc/hexanes to 100% EtOAc afforded 2-ethoxy-1-(1-methyl-pyrazol-4-yl)-ethanone. ¹H NMR (500 MHz, CDCl₃): δ 8.07 (s, 1H), 8.03 (s, 1H), 4.38 (s, 2H), 3.96 (s, 3H), 3.62 (q, 2H), 1.29 (t, 3H).

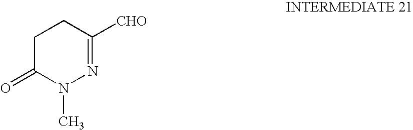

### Step A: 3-Hydroxymethyl-1-methyl-6-oxo-1,4,5,6-tetrahydropyridazine

1-Methyl-6-oxo-1,4,5,6-tetrahydropyridazine-3-carboxylic acid (200 mg, 1.281 mmol) was dissolved in THF (2.0 mL). Triethylamine (0.179 mL, 1.281 mmol) was added and the reaction was cooled in an ice bath. Ethyl chloroformate (0.168 mL, 1.281 mmol) was added all at once. A precipitate was formed and the mixture was stirred at the ice bath temp. for 15 min. NaBH₄ (121 mg, 3.2 mmol) in water (1.0 mL) was added, resulting in vigorous gas evolution. The ice bath was removed and the reaction was stirred at rt for 2 h. Some water was added and the mixture was extracted three times with CH₂Cl₂. The combined organic extracts were washed with brine. The aqueous layer was evaporated to dryness and triturated with CH₂Cl₂, with stirring for 15 mM. The mixture was filtered and the solids were re-treated with CH₂Cl₂ with stirring for 10 min. The mixture was filtered, all the CH₂Cl₂ extracts combined and evaporated to dryness. The residue was dried under high vacuum at rt to afford the crude product as a colorless oil. The product was purified by flash chromatography on silica gel (1¼″×3¾″) eluting with 12:8:2 hexane-EtOAc-MeOH to afford 3-hydroxymethyl-1-methyl-6-oxo-1,4,5,6-tetrahydropyridazine as a colorless oil. MS: [M+H]+=143. ¹H-NMR (500 MHz, CDCl₃): δ CH₂—O (4.31, s, N—CH₃ (3.4, s, 3H), CH₂'s of ring (2.54, m, 4H), OH+H₂O (2.2, broad baseline peak, ˜2H).

### Step B: 1-Methyl-6-oxo-1,4,5,6-tetrahydropyridazine-3-carboxaldehyde

Oxalyl chloride (382 μL, 4.36 mmol) was dissolved in CH₂Cl₂ (4.0 mL) and cooled to −70°. DMSO (619 μL, 8.73 mmol) was added over a few min, resulting in vigorous gas evolution. The reaction mixture was stirred at −70° for 20 min, and a solution of 3-hydroxymethyl-1-methyl-6-oxo-1,4,5,6-tetrahydropyridazine (564 mg, 3.97 mmol) in CH₂Cl₂ (6 mL) was then added over 5 min. A precipitate formed and the mixture was stirred at −70° for an additional 40 min. Triethylamine (2.76 mL, 19.84 mmol) was then added, the ice bath removed, and the reaction warmed to rt. The mixture was diluted with CH₂Cl₂ and a small amount of water was added along with some brine. The layers were separated and the aqueous layer extracted twice with CH₂Cl₂ containing a small amount of MeOH. The combined extracts were dried over anhydrous MgSO₄, filtered, and concentrated by rotoevaporation. The product was purified by flash chromatography on silica gel eluting with hexane-EtOAc-MeOH (12:8:2) to afford 1-methyl-6-oxo-1,4,5,6-tetrahydropyridazine-3-carboxaldehyde as a pale yellow solid. MS: [M+H]+=141.

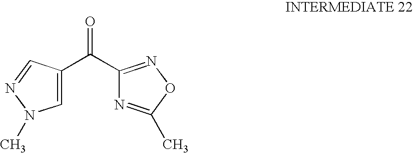

### 1-Methyl-pyrazol-4-yl-5-methyl-1,2,4-oxadiazol-3-yl ketone

To a solution of 1-methyl-4-iodo-1H-pyrazole (3 g, 14.42 mmol) in THF (40 mL) was added isopropylmagnesium chloride 2.0M in THF (8.00 mL, 16.01 mmol) at 0° C. The mixture was stirred at 0° C. for 1 h, cooled to −78° C., and N-methoxy-N-methyl-5-methyl-1,2,4-oxadiazole-3-carboxamide (prepared from the acid chloride of 5-methyl-1,2,4-oxadiazole-3-carboxylic acid and N-methoxy-N-methylamine hydrochloride according to the procedure described for the preparation of Intermediate 19, Step A) (3.21 g, 18.75 mmol) was added. The mixture was slowly warmed to RT in 1.5 h. The reaction was cooled to −78° C. and quenched by slow dropwise addition of a saturated solution of ammonium chloride and warmed to RT. The reaction was stored in the cold overnight. The reaction was diluted with cold 1N aqueous HCl, extracted four times with EtOAc. The combined organic layers were washed with brine and dried over anhydrous Na₂SO₄. The product was purified by silica gel chromatography eluting with a gradient of 10% EtOAc in hexanes to 100% EtOAc to afford 1-methyl-pyrazol-4-yl 5-methyl-1,2,4-triazol-3-yl ketone. ¹H NMR (500 MHz, CDCl₃): δ 8.41 (s, 1H), 8.29 (s, 1H), 3.99 (s, 3H), 2.71 (s, 3H).

### Example 1

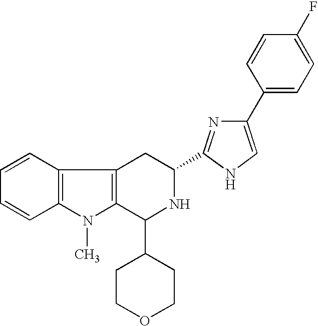

### (3R)-1-(Tetrahydro-2H-pyran-4-yl)-3-(4-(4-fluorophenyl)-1H-imidazol-2-yl)-9-methyl-2,3,4,9-tetrahydro-1H-β-carboline

A 25 mL one-neck round bottom flask was charged with tert-butyl 1(R)-2-(1-methyl-1H-indol-3-yl)-1-(4-(4-fluorophenyl)-1H-imidazol-2-yl)ethylcarbamate (Intermediate 3) (106 mg, 0.244 mmol), methylene chloride (1 mL) and TFA (0.5 mL). The mixture was stirred at rt for 30 min. Tetrahydro-2H-pyranyl-4-carboxaldehyde (55.7 mg, 0.488 mmol) was then added and the resulting reaction mixture was stirred at rt for 15 h. The reaction mixture was concentrated and the residue was partitioned between water and ethyl acetate. The aqueous layer was made basic with saturated aqueous NaHCO₃ and worked up by extraction. The product was then purified by PrepTLC (2000 nm, 3:2 ethyl acetate/hexanes) to afford (3R)-1-(tetrahydro-2H-pyran-4-yl)-3-(4-(4-fluorophenyl)-1H-imidazol-2-yl)-9-methyl-2,3,4,9-tetrahydro-1H-β-carboline. LC-MS: m/z 431 (M+H)⁺. ¹H NMR (CDCl₃, 500 MHz) δ (ppm): 7.70 (2H, br), 7.56 (1H, d, J=8 Hz), 7.30 (1H, d, J=8 Hz), 7.24 (1H, t, J=8 Hz), 7.14 (1H, t, 7.5 Hz), 7.07 (2H, t, 8.5 Hz), 4.59 (1H, m), 4.06 (2H, m), 3.93 (2H, dd), 3.44 (1H, m), 3.32 (2H, m), 3.05 (1H, dd), 2.14 (1H, m), 1.67 (3H, m).

### Example 2

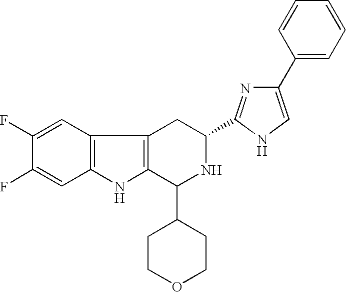

### (3R)-6,7-Difluoro-3-(4-phenyl-1H-imidazol-2-yl)-1-(tetrahydro-2H-pyran-4-yl)-2,3,4,9-tetrahydro-1H-β-carboline

A mixture of the faster-eluting enantiomer of 2-(5,6-difluoro-1H-indol-3-yl)-1-(4-phenyl-1H-imidazol-2-yl)-1-ethylcarbamate (Intermediate 5) (0.02 g, 0.046 mmol) and trifluoroacetic acid (0.039 mL, 0.502 mmol) in dichloromethane (1 mL) was stirred at rt for 30 min. The solvent was removed under reduced pressure. To the residue was added tetrahydro-2H-pyranyl-4-carboxaldehyde (0.01 g, 0.091 mmol) and dichloromethane (1 mL). The resulting mixture was stirred at rt for 2 h. The reaction mixture was filtered and concentrated to dryness. The residue was purified by HPLC to give the title compound. ¹H NMR (500 MHz, CD₃OD): δ 7.81˜7.74 (m, 3H), 7.51˜7.7.48 (m, 2H), 7.45˜7.41 (m, 1H), 7.31˜7.27 (m, 1H), 7.23˜7.20 (m, 1H), 4.71 (dd, 1H), 4.59 (s, 1H), 4.06 (dd, 1H), 3.97 (dd, 1H), 3.53 (t, 1H), 3.45 (t, 1H), 3.28 (d, 1H), 3.18 (qt, 1H), 2.44 (t, 1H), 1.92˜1.86 (m, 1H), 1.79˜1.72 (m, 2H), 1.23 (d, 1H). LC-MS: m/z 435 (M+H)⁺ (2 min).

### Example 3

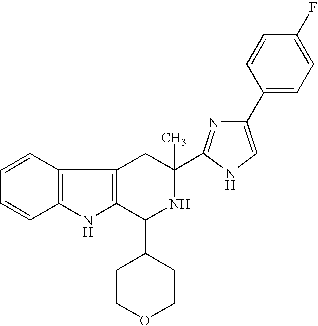

### 3-(4-(4-Fluoro-phenyl)-1H-imidazol-2-yl)-3-methyl-1-(tetrahydro-2H-pyran-4-yl)-2,3,4,9-tetrahydro-1H-β-carboline

To a suspension of the faster-eluting enantiomer from Intermediate 9 (100 mg, 0.230 mmol) in CH₂Cl₂ (3 mL) was added TFA (2 mL). The reaction was stirred at rt for 1 h and then concentrated. The resulting material was dissolved in CH₂Cl₂ (5 mL) and 4-tetrahydro-2H-pyranyl-4-carboxaldehyde (52.5 mg, 0.460 mmol) was added. The reaction was stirred overnight at rt. The material was concentrated to afford a residue, which was purified by preparative TLC eluting with the following solvent system as mobile phase: 5% (10% NH₄OH/90% CH₃OH)/95% CH₂Cl₂. Chiral HPLC resolution of the diastereoisomers was carried out with a ChiralCel® OD® column (4.6×250 mm), flow rate at 0.5 mL/min of 15% ethanol in heptane, and UV detection at 220 nm. The retention times of the faster-eluting diastereoisomer and the slower-eluting diastereoisomer were 11.7 min and 22.9 min, respectively. ¹H NMR of the faster-eluting isomer: (500 MHz, CD₃OD): δ 7.80 (m, 2H), 7.48 (m, 2H), 7.39 (m, 1H), 7.16 (m, 3H), 7.04 (t, 1H), 4.43, (s, 1H), 4.07, (dd, 1H), 3.98 (dd, 1H), 3.53 (t, 1H), 3.46 (t, 1H), 3.26, (m, 2H), 2.39 (m, 1H), 1.84 (m, 2H), 1.66 (s, 3H), 1.36 (m, 2H). LC-MS: m/z 431.06 (M+H)⁺ (2.72 min). LC-MS of the slower-eluting isomer: m/z 431.06 (M+H)⁺ (2.62 min).

### Example 4

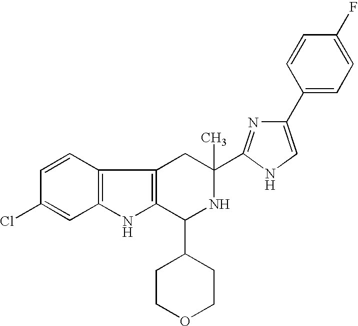

### 7-Chloro-3-(4-(4-fluoro-phenyl)-1H-imidazol-2-yl)-3-methyl-1-(tetrahydro-2H-pyran-4-yl)-2,3,4,9-tetrahydro-1H-β-carboline

A mixture of the faster-eluting enantiomer from Intermediate 8, Step H (32 mg, 0.068 mmol) and trifluoroacetic acid (86 mg, 0.751 mmol) in dichloromethane (1 ml) was stirred at rt for 30 min. The solvent was removed under reduced pressure. To the residue was added 4-tetrahydro-2H-pyranyl-4-carboxaldehyde (23.37 mg, 0.205 mmol) and dichloromethane (1 mL). The resulting mixture was stirred at rt for 2 h. The reaction mixture was partitioned between ethyl acetate and saturated NaHCO₃ solution (30 mL/10 mL). The aqueous layer was extracted twice with ethyl acetate (20 mL). The combined organic extracts were washed with brine, dried over anhydrous magnesium sulfate, filtered and concentrated to dryness. The residue was purified by preparative TLC on silica gel eluting with ethyl acetate to give each individual diastereoisomer.

¹H NMR of the faster-eluting diastereoisomer: (500 MHz, CDCl₃): δ 8.24 (s, 1H), 7.70 (s, br, 1H), 7.33 (d, 2H), 7.22 (s, 1H), 7.08˜7.05 (m, 3H), 4.15 (s, 1H), 4.05 (dd, 1H), 3.95 (dd, 1H), 3.44 (t, 1H), 3.34 (t, 1H), 3.12 (qt, 2H), 1.83 (qt, 1H), 1.61 (d, 2H), 1.52 (s, 3H), 1.30˜1.26 (m, 2H). LC-MS: m/z 442 (M+H)⁺ (2 min).

¹H NMR of the slower-eluting diastereoisomer: (500 MHz, CDCl₃): δ 8.04 (s, 1H), 7.56˜7.50 (m, 1H), 7.41 (d, 1H), 7.25 (s, 1H), 7.12˜7.09 (m, 1H), 7.03˜6.99 (m, 2H), 6.96 (s, 1H), 4.07 (d, 1H), 3.99 (d, 1H), 3.91 (s, 1H), 3.53˜3.35 (m, 3H), 2.91 (d, 1H), 1.99 (d, 1H), 1.80 (d, 1H), 1.72 (s, 3H), 1.61 (d, 1H), 1.34˜1.25 (m, 2H). LC-MS: m/z 465 (M+H)⁺ (2 min).

### Example 5

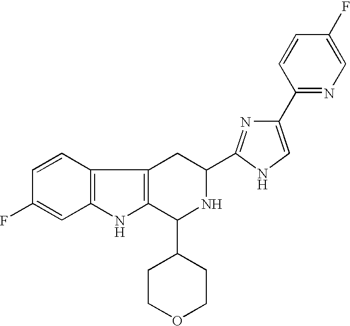

### 7-Fluoro-3-(4-(5-fluoro-pyridin-2-yl)-1H-imidazol-2-yl)-1-(tetrahydro-2H-pyran-4-yl)-2,3,4,9-tetrahydro-1H-β-carboline

To a stirred solution of the faster-eluting enantiomer of tert-butyl 2-(6-fluoro-1H-indol-3-yl)-1-(4-(4-fluoropyridin-2-yl)-1H-imidazol-2-yl)-1-ethylcarbamate from Intermediate 6, Step G (60 mg, 0.137 mmol) in anhydrous dichloromethane (2 mL) was added TFA (2 mL). The mixture was stirred at rt for 30 min and then evaporated. The residue was dissolved in anhydrous dichloromethane (2 mL) and tetrahydro-2H-pyran-4-carboxaldehyde (31.2 mg, 0.273 mmol) was added. The mixture was stirred at rt overnight. After work-up, the crude product was purified by reverse-phase HPLC to yield 7-fluoro-3-(4-(5-fluoro-pyridin-2-yl)-1H-imidazol-2-yl)-1-(tetrahydro-2H-pyran-4-yl)-2,3,4,9-tetrahydro-1H-β-carboline. ¹H NMR (500 MHz, CDCl₃): δ 8.60-8.45 (1H), 8.10-7.80 (2H), 7.78-7.68 (1H), 7.55-7.42 (1H), 7.15-7.05 (1H), 6.95-6.82 (1H), 4.98-4.80 (2H), 4.15-4.00 (2H), 3.60-3.30 (4H), 2.60-2.50 (1H), 1.95-1.78 (3H), 1.42-1.35 (1H). LC-MS found for C₂₄H₂₃F₂N₅₀: m/z 436 (M+H)⁺ (1.01 min).

### Example 6

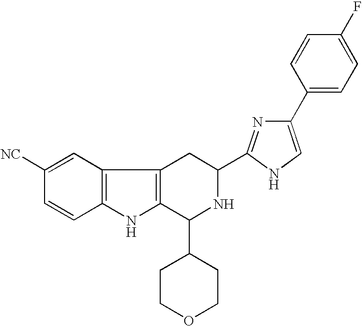

### 6-Cyano-3-(4-(4-fluorophenyl)-1H-imidazol-2-yl)-1-(tetrahydro-2H-pyran-4-yl)-2,3,4,9-tetrahydro-1H-β-carboline

### Step A: 6-Bromo-3-(4-(4-fluorophenyl)-1H-imidazol-2-yl)-1-(tetrahydro-2H-pyran-4-yl)-2,3,4,9-tetrahydro-1H-β-carboline

A mixture of the faster-eluting enantiomer of tert-butyl 2-(5-bromo-1H-indol-3-yl)-1-(4-(4-fluorophenyl)-1H-imidazol-2-yl)-1-ethylcarbamate from Intermediate 4, Step D (0.056 g, 0.112 mmol) and trifluoroacetic acid (0.095 mL, 1.234 mmol) in dichloromethane (1 mL) was stirred at rt for 30 min. The solvent was then removed under reduced pressure. To the residue was added tetrahydropyranyl-4-carboxaldehyde (0.026 g, 0.224 mmol) and dichloromethane (1 mL). The resulting mixture was stirred at rt for 2 h. The reaction mixture was filtered and concentrated to dryness, and the residue was purified by HPLC to give 6-bromo-3-(4-(4-fluorophenyl)-1H-imidazol-2-yl)-1-(tetrahydro-2H-pyran-4-yl)-2,3,4,9-tetrahydro-1H-β-carboline as a single diastereoisomer. ¹H NMR (500 MHz, CD₃OD): δ 7.82˜7.79 (m, 2H), 7.77 (s, 1H), 7.62 (s, 1H), 7.29 (t, 1H), 7.24˜7.21 (m, 3H), 4.81 (dd, 1H), 4.72 (s, 1H), 4.06 (dd, 1H), 3.97 (dd, 1H), 3.52 (t, 1H), 3.45 (t, 1H), 3.36˜3.24 (m, 2H), 2.51 (t, 1H), 1.86 (qt, 1H), 1.80˜1.72 (m, 2H), 1.27 (d, 1H). LC-MS: m/z 495 (M+H)⁺ (2 min).

### Step B: 6-Cyano-3-(4-(4-fluorophenyl)-1H-imidazol-2-yl)-1-(tetrahydro-2H-pyran-4-yl)-2,3,4,9-tetrahydro-1H-β-carboline

A mixture of 6-bromo-3-(4-(4-fluorophenyl)-1H-imidazol-2-yl)-1-(tetrahydro-2H-pyran-4-yl)-2,3,4,9-tetrahydro-1H-β-carboline (50 mg, 0.069 mmol), zinc dust (1.62 mg, 0.025 mmol), zinc cyanide (19.48 mg, 0.166 mmol), 1,1′-bis(diphenylphosphino)-ferrocene (6.13 mg, 0.011 mmol), tris(dibenzylideneacetone)dipalladium (5.06 mg, 5.53 μmol), and anhydrous N,N-dimethylacetamide (1 mL) in a heavy wall pyrex vial was exposed to microwave irradiation at 130° C. for 1 h. The reaction mixture was partitioned between ethyl acetate and saturated aq. NaHCO₃ solution (30 mL/20 mL). The aqueous layer was extracted twice with ethyl acetate (30 mL). The combined organic extracts were washed with brine, dried over anhydrous magnesium sulfate, filtered and concentrated to dryness. The residue was purified by HPLC to give 6-cyano-3-(4-(4-fluorophenyl)-1H-imidazol-2-yl)-1-(tetrahydro-2H-pyran-4-yl)-2,3,4,9-tetrahydro-1H-β-carboline. ¹H NMR (500 MHz, CD₃OD): δ 7.91 (s, 1H), 7.82˜7.78 (m, 3H), 7.51 (d, 1H), 7.41 (d, 1H), 7.25 (t, 2H), 4.66 (dd, 1H), 4.58 (s, 1H), 4.06 (dd, 1H), 3.96 (dd, 1H), 3.53 (t, 1H), 3.45 (t, 1H), 3.34 (d, 1H), 3.16 (t, 1H), 2.48 (t, 1H), 1.95˜1.86 (m, 1H), 1.81˜1.72 (m, 2H), 1.18 (d, 1H). LC-MS: m/z 442 (M+H)⁺ (2 min).

### Example 7

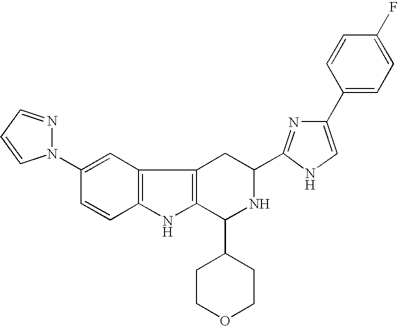

### 6-(Pyrazol-1-yl)-3-(4-(4-fluorophenyl)-1H-imidazol-2-yl)-1-(tetrahydro-2H-pyran-4-yl)-2,3,4,9-tetrahydro-1H-β-carboline

### Step A: 6-Bromo-3-(4-(4-fluorophenyl)-1-(tert-buyloxycarbonyl)-1H-imidazol-2-yl)-2,9-bis(tert-butyloxycaryl)-1-(tetrahydro-2H-pyran-4-yl)-2,3,4,9-tetrahydro-1H-β-carboline

A mixture of 6-bromo-3-(4-(4-fluorophenyl)-1H-imidazol-2-yl)-1-(tetrahydro-2H-pyran-4-yl)-2,3,4,9-tetrahydro-1H-β-carboline from Example 6, Step A (0.3 g, 0.606 mmol), triethylamine (0.508 mL, 3.63 mmol), di-tert-butyl dicarbonate (0.423 g, 1.938 mmol), and a catalytic amount of DMAP in anhydrous dichloromethane (2 mL) was stirred at rt for 16 h. The solvent was removed under reduced pressure and the residue was purified by preparative TLC eluting with 20% ethyl acetate in hexane to give 6-bromo-3-(4-(4-fluorophenyl)-1-(tert-buyloxycarbonyl)-1H-imidazol-2-yl)-2,9-bis(tert-butyloxycarbonyl)-1-(tetrahydro-2H-pyran-4-yl)-2,3,4,9-tetrahydro-1H-β-carboline. LC-MS: m/z 797 (M+H)⁺ (2 min).

### Step B: 6-(Pyrazol-1-yl)-3-(4-(4-fluorophenyl)-1H-imidazol-2-yl)-2-(tert-butyloxycarbonyl)-1-(tetrahydro-2H-pyran-4-yl)-2,3,4,9-tetrahydro-1H-β-carboline

A mixture of 6-bromo-3-(4-(4-fluorophenyl)-1-(tert-buyloxycarbonyl)-1H-imidazol-2-yl)-2,9-bis(tert-butyloxy carbonyl)-1-(tetrahydro-2H-pyran-4-yl)-2,3,4,9-tetrahydro-1H-β-carboline (40 mg, 0.05 mmol), pyrazole (11.71 mg, 0.25 mmol), copper iodide (47.9 mg, 0.25 mmol), (1R,2R′)—N,N′-dimethyl-1,2-cyclohexanediamine (35.8 mg, 0.25 mmol), potassium carbonate (34.7 mg, 0.25 mmol), and anhydrous acetonitrile (1 mL) in a heavy-wall pyrex vial was subjected to microwave irradiation at 150° C. for 2 h. The reaction mixture was filtered through celite and concentrated under reduced pressure. The residue was partitioned between ethyl acetate and saturated NaHCO₃ solution (30 mL/20 mL). The aqueous layer was extracted twice with ethyl acetate (30 mL). The combined organic extracts were washed with brine, dried over anhydrous magnesium sulfate, filtered and concentrated to dryness to yield 6-(pyrazol-1-yl)-3-(4-(4-fluorophenyl)-1H-imidazol-2-yl)-2-(tert-butyloxycarbonyl)-1-(tetrahydro-2H-pyran-4-yl)-2,3,4,9-tetrahydro-1H-β-carboline which was used in the next step without further purification. LC-MS: m/z 583 (M+Na)⁺ (2 min).

### Step C: 6-(Pyrazol-1-yl)-3-(4-(4-fluorophenyl)-1H-imidazol-2-yl)-1-(tetrahydro-2H-pyran-4-yl)-2,3,4,9-tetrahydro-1H-β-carboline

A mixture of 6-(pyrazol-1-yl)-3-(4-(4-fluorophenyl)-1H-imidazol-2-yl)-2-(tert-butyloxycarbonyl)-1-(tetrahydro-2H-pyran-4-yl)-2,3,4,9-tetrahydro-1H-β-carboline (40 mg, 0.069 mmol), concentrated HCl (1 mL), and methanol (1 mL) was heated at 40° C. for 2 h. The reaction mixture was concentrated under reduced pressure, and the residue purified by HPLC to give 6-(pyrazol-1-yl)-3-(4-(4-fluorophenyl)-1H-imidazol-2-yl)-1-(tetrahydro-2H-pyran-4-yl)-2,3,4,9-tetrahydro-1H-β-carboline. ¹H NMR (500 MHz, CD₃OD): δ 8.11 (s, 1H), 7.82˜7.79 (m, 3H), 7.74 (d, 1H), 7.69 (s, 1H), 7.47 (d, 2H), 7.23 (t, 2H), 6.50 (d, 1H), 4.73 (dd, 1H), 4.67 (s, 1H), 4.07 (dd, 1H), 3.98 (dd, 1H), 3.54 (t, 1H), 3.49 (t, 1H), 3.38 (dd, 1H), 2.50 (t, 1H), 1.89 (qt, 1H), 1.78-1.76 (m, 2H), 1.28 (d, 1H). LC-MS: m/z 483 (M+H)⁺ (2 min).

### Example 8

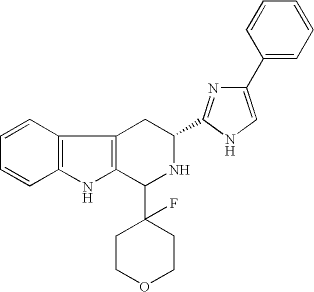

### (3R)-1-(4-Fluoro-tetrahydro-2H-pyran-4-yl)-3-(4-phenyl-1H-imidazol-2-yl)-2,3,4,9-tetrahydro-1H-β-carboline

### Step A: 4-Fluoro-tetrahydro-2H-pyran-4-carboxaldehyde

A solution of DIPEA (6.12 mL, 35.0 mmol) in dichloromethane (100 mL) was cooled in ice-water bath. To this solution was added trimethylsilyl trifluoromethanesulfonate (6.33 mL, 35.0 mmol) followed by a solution of tetrahydro-2H-pyranyl-4-carboxaldehyde (2 g, 17.52 mmol) in dichloromethane (100 mL). Upon completion of the addition, the ice-water bath was removed. The reaction was stirred at RT for 2 h. The reaction was concentrated, treated with hexane (200 mL) and kept at RT for 1 h. The mixture was filtered and filtrate concentrated to yield the crude TMS ether. To a solution of the crude TMS ether in dichloromethane (100 mL) was added N-fluorobenzenesulfonimide (9.95 g, 31.5 mmol) in dichloromethane (100 mL) at 0° C. via an addition funnel. After 3 h, the reaction mixture containing 4-fluoro-tetrahydro-2H-pyran-4-carboxaldehyde was used as is in the next step.

### Step B: (3R)-1-(4-Fluoro-tetrahydro-2H-pyran-4-yl)-3-(4-phenyl-1H-imidazol-2-yl)-2,3,4,9-tetrahydro-1H-β-carboline

tert-Butyl (1R)-2-(1H-indol-3-yl)-1-(4-phenyl-1H-imidazol-2-yl)-1-ethylcarbamate (Intermediate 1) (305 mg, 0.757 mmol) was treated with dichloromethane (3 mL) followed by trifluoroacetic acid (10 mL). The mixture was stirred at RT for 30 min and was then concentrated. Crude 4-fluoro-tetrahydro-2H-pyran-4-carboxaldehyde from Step A (approximately 200 mg, 1.514 mmol) in CH₂Cl₂ (25 mL) was added. After stirring at RT for 2 h, one-third of the reaction mixture (8 mL) was transferred to a large cartridge containing a half-inch of a thoroughly mixed solid mixture of silica gel and NaHCO₃. Flash column chromatography on silica gel eluting with a gradient of 100% dichloromethane to 100% acetone afforded (3R)-1-(4-fluoro-tetrahydro-2H-pyran-4-yl)-3-(4-phenyl-1H-imidazol-2-yl)-2,3,4,9-tetrahydro-1H-β-carboline. ¹H NMR (500 MHz, CDCl₃): δ 8.45 (s, 1H), 7.70 (d, 2H), 7.40 (d, 1H), 7.36 (m, 3H), 7.26 (t, 1H), 7.19 (t, 1H), 7.09 (t, 1H), 4.37 (dd, 1H), 4.32 (d, 1H), 3.82 (m, 1H), 3.70 (m, 2H), 3.62 (t, 1H), 3.19 (d, 1H), 2.97 (t, 1H), 2.06 (m, 1H), 1.83 (m, 1H), 1.57 (t, 1H), 1.21 (t, 1H). LC-MS: m/z 417.06 (M+H)⁺ (2.68 min).

### Example 9

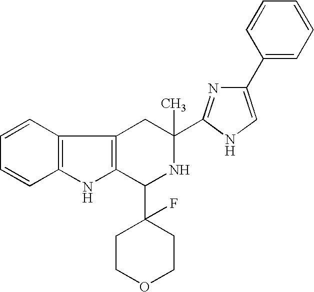

### 1-(4-Fluoro-tetrahydro-2H-pyran-4-yl)-3-methyl-3-(4-phenyl-1H-imidazol-2-yl)-2,3,4,9-tetrahydro-1H-β-carboline

The faster-eluting enantiomer of tert-butyl 2-(1H-indol-3-yl)-1-(4-(4-fluorophenyl)-1H-imidazol-2-yl)-1-methyl-1-ethylcarbamate (Intermediate 11) (315 mg, 0.757 mmol) was dissolved in CH₂Cl₂ (3 mL) followed by TFA (10 mL). The mixture was stirred at RT for 30 min and then concentrated. Crude 4-fluoro-tetrahydro-2H-pyran-4-carboxaldehyde from Example 8, Step A (about 10 mg/mL) (200 mg, 1.514 mmol) in CH₂Cl₂ (25 mL) was added. The reaction mixture (8 mL) was transferred to a large cartridge containing a half-inch of a thoroughly mixed solid mixture of silica gel and NaHCO₃. Flash column chromatography on silica gel eluting with a gradient of 100% dichloromethane to 100% acetone afforded 1-(4-fluoro-tetrahydro-2H-pyran-4-yl)-3-methyl-3-(4-phenyl-1H-imidazol-2-yl)-2,3,4,9-tetrahydro-1H-β-carboline. LC-MS: m/z 431.14 (M+H)⁺ (2.79 min).

### Example 10

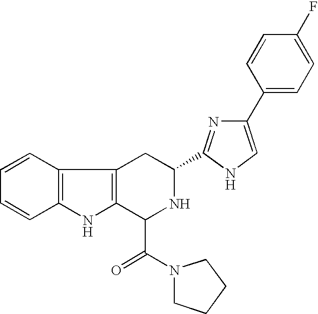

### (3R)-3-[4-(4-Fluorophenyl)-1H-imidazol-2-yl]-2,3,4,9-tetrahydro-1H-β-carboline-1-carboxylic acid, pyrrolidine amide

### Step A: (3R)-3-[4-(4-Fluorophenyl)-1H-imidazol-2-yl]-2,3,4,9-tetrahydro-1H-beta-carboline-1-carboxylic acid

tert-Butyl (1R)-2-(1H-indol-3-yl)-1-(4-(4-fluorophenyl)-1H-imidazol-2-yl)-1-ethylcarbamate (Intermediate 10) (1 g, 2.378 mmol) was treated with CH₂Cl₂ (10 mL) followed by trifluoroacetic acid (4 mL). The mixture was stirred at RT for 1 h and was then concentrated. The residue was treated with ethyl acetate (6 mL). To this mixture was added dropwise glyoxylic acid monohydrate (0.263 g, 2.85 mmol) in water (3 mL). The pH of the mixture was adjusted to 5 with 10% aq. K₂CO₃. The mixture was stirred at RT overnight. The mixture was purified by reverse-phase HPLC on a C-18 column eluting with a gradient of 10% to 100% acetonitrile (containing 0.1% trifluoroacetic acid) in water (containing 0.1% trifluoroacetic acid) to afford (3R)-3-[4-(4-fluorophenyl)-1H-imidazol-2-yl]-2,3,4,9-tetrahydro-1H-beta-carboline-1-carboxylic acid as an approximately 2:1 mixture of diastereoisomers. LC-MS: m/z 377.15 (M+H)⁺ (2.43 min).

### Step B: (3R)-3-[4-(4-Fluorophenyl)-1H-imidazol-2-yl]-2,3,4,9-tetrahydro-1H-β-carboline-1-carboxylic acid, pyrrolidine amide

A mixture of (3R)-3-[4-(4-fluorophenyl)-1H-imidazol-2-yl]-2,3,4,9-tetrahydro-1H-β-carboline-1-carboxylic acid (31 mg, 0.051 mmol), N,N,N′,N′-tetramethyl-O-(7-azabenzotriazol-1-yl)uronium hexafluorophosphate (98 mg, 0.256 mmol), 1-hydroxy-7-azabenzotriazole (34.9 mg, 0.256 mmol) and pyrrolidine (0.064 mL, 0.769 mmol) in CH₂Cl₂ (2 mL) was stirred at RT overnight. The mixture was then concentrated. The residue was subjected to preparative TLC on silica gel eluting twice with 200:10:1 CH₂Cl₂/MeOH/NH₄OH to afford (3R)-3-[4-(4-fluorophenyl)-1H-imidazol-2-yl]-2,3,4,9-tetrahydro-1H-β-carboline-1-carboxylic acid, pyrrolidine amide. LC-MS: m/z 430.19 (M+H)⁺ (2.75 min).

### Example 11

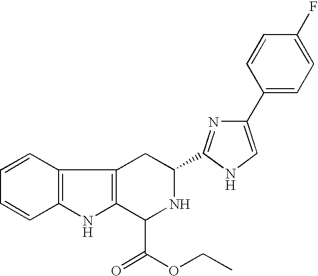

### (3R)-3-[4-(4-Fluorophenyl)-1H-imidazol-2-yl]-2,3,4,9-tetrahydro-1H-β-carboline-1-carboxylic acid, ethyl ester

To a solution of (1R)-1-[4-(4-fluorophenyl)-1H-imidazol-2-yl]-2-(1H-indol-3-yl)ethanamine hydrochloride (4 g, 11.2 mmol) [prepared by treatment of tert-butyl (1R)-2-(1H-indol-3-yl)-1-(4-(4-fluorophenyl)-1H-imidazol-2-yl)-1-ethylcarbamate (Intermediate 10) with hydrochloric acid] in EtOH (10 mL) was added glyoxylic acid, ethyl ester in toluene (2.74 mL, 13.45 mmol). The mixture was stirred at rt overnight, diluted with EtOAc, washed with 1 N NaOH, brine, dried and concentrated. The crude residue was purified by column chromatography on silica gel to give (3R)-3-[4-(4-fluorophenyl)-1H-imidazol-2-yl]-2,3,4,9-tetrahydro-1H-β-carboline-1-carboxylic acid, ethyl ester as a mixture of two diastereoisomers in a 2:1 ratio. This mixture was further purified by preparative TLC and a small amount of the more polar, slower eluting compound was isolated in pure form. ¹H NMR (500 MHz, CD₃OD): δ 7.73 (br t, 2H), 7.46 (d, 1H), 7.35 (m, 2H), 7.11 (m, 3H), 7.00 (m, 1H), 4.92 (s, 1H), 4.65 (dd, 1H), 4.28 (m, 2H), 3.16 (dd, 1H), 3.01 (dd, 1H), 1.32 (t, 3H). LC-MS: m/z 405 (M+1)⁺ at 2.71 min.

### Example 12

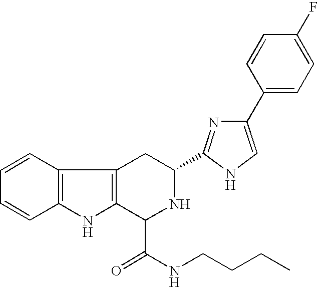

### (3R)-3-[4-(4-Fluorophenyl)-1H-imidazol-2-yl]-2,3,4,9-tetrahydro-1H-β-carboline-1-carboxylic acid, n-butyl amide

### Step A: (3R)-3-[4-(4-Fluorophenyl)-1H-imidazol-2-yl]-2,3,4,9-tetrahydro-1H-β-carboline-1-carboxylic acid, methyl ester

To a solution of (1R)-1-[4-(4-fluorophenyl)-1H-imidazol-2-yl]-2-(1H-indol-3-yl)ethanamine hydrochloride (1 g, 2.8 mmol) in MeOH (20 mL) was added glyoxylic acid monohydrate (0.31 g, 3.36 mmol). The mixture was stirred at rt overnight. It was then diluted with EtOAc, washed with 1 N NaOH, brine, dried and concentrated. The crude residue was purified by column chromatography on silica gel eluting with a gradient of 5-100% ethyl acetate in hexanes to give (3R)-3-[4-(4-fluorophenyl)-1H-imidazol-2-yl]-2,3,4,9-tetrahydro-1H-β-carboline-1-carboxylic acid, methyl ester. LC-MS: m/z 391 (M+H)⁺ at 2.6 min.

### Step B: (3R)-3-[4-(4-fluorophenyl)-1H-imidazol-2-yl]-2,3,4,9-tetrahydro-1H-β-carboline-1-carboxylic acid, n-butyl amide

(3R)-3-[4-(4-Fluorophenyl)-1H-imidazol-2-yl]-2,3,4,9-tetrahydro-1H-β-carboline-1-carboxylic acid, methyl ester (150 mg, 0.384 mmol) was mixed with n-butylamine (2 mL). The mixture was then stirred at 60° C. for 5 h. The reaction mixture was diluted with EtOAc, washed with water, brine, dried and concentrated. The residue was purified by preparative TLC eluting with 50% acetone in hexanes to afford the two diastereoisomers of (3R)-3-[4-(4-fluorophenyl)-1H-imidazol-2-yl]-2,3,4,9-tetrahydro-1H-β-carboline-1-carboxylic acid, n-butyl amide. ¹H NMR of the less polar product: (500 MHz, CD₃OD): δ 7.75 (br 2H), 7.50 (d, 1H), 7.37 (d, 2H), 7.10 (m, 3H), 7.00 (1H), 4.73 (s, 1H), 4.23 (dd, 1H), 3.28 (m, 1H), 3.15 (t, 1H), 3.00 (dd, 1H), 1.57-1.31 (m, 6H), 0.93 (t, 3H). LC-MS m/z 421 (M+1)⁺ at 2.66 min. ¹H NMR of the more polar product: (500 MHz, CD₃OD): δ 7.75 (br 2H), 7.42 (d, 1H, 7.38 (br, 1H), 7.36 (d, 1H), 7.10 (m, 3H), 6.99 (t, 1H), 4.89 (s, 1H), 4.40 (dd, 1H), 3.27 (m, 2H), 1.52 (m, 2H), 1.33 (m, 1H), 0.89 (t, 3H). LC-MS: m/z 421 (M+1)⁺ at 2.66 min.

### Example 13

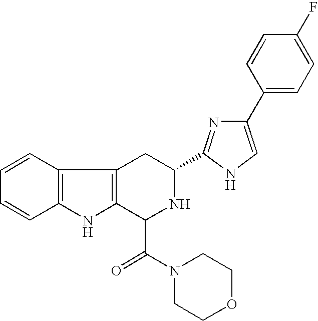

### (3R)-3-[4-(4-Fluorophenyl)-1H-imidazol-2-yl]-2,3,4,9-tetrahydro-1H-β-carboline-1-carboxylic acid, 4-morpholinyl amide

To a solution of (3R)-3-[4-(4-fluorophenyl)-1H-imidazol-2-yl]-2,3,4,9-tetrahydro-1H-β-carboline-1-carboxylic acid, ethyl ester (180 mg, 0.445 mmol) in ethanol were added 4-morpholine HCl (122 mg, 0.89 mmol) and triethylamine (0.248 mL). The mixture was stirred under microwave irradiation at 130° C. for 4.5 h, diluted with EtOAc, washed with water, brine, dried and concentrated. The residue was purified by preparative TLC eluting with 50% acetone in hexanes to give each individual diastereoisomer.

¹H NMR of the less polar product: (500 MHz, CD₃OD): δ 7.75 (br t, 2H), 7.49 (d, 1H), 7.36 (d, 1H), 7.35 (s, 1H), 7.10 (m, 3H), 7.00 (t, 1H), 4.73 (s, 1), 4.25 (dd, 1H), 3.93 (m, 3H), 3.46 (m, 2H), 334 (m, 1H), 2.95 (m, 1H), 1.89 (m, 1H), 1.77 (m, 1H), 1.64 (m, 2H). LC-MS: m/z 460 (M+1)⁺ at 2.57 min. ¹H NMR of the more polar product: (500 MHz, CD₃OD): δ 7.74 (br, 2H), 7.48, 7.41 (d, 1H), 7.36 (d, 2H), 7.10 (m, 3H), 7.00 (m, 1H), 4.72 (s, 1H), 4.36, 4.25 (dd, 1H), 3.93 (m, 3H), 3.43 (m, 2H), 3.11-2.95 (m, 2H), 1.87 (m, 1H), 1.68 (m, 3H). LC-MS: m/z 460 (M+1)⁺ at 2.63 min.

### Example 14

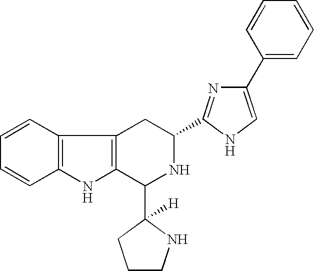

### (3R)-3-[4-Phenyl-1H-imidazol-2-yl]-1-((2S)-pyrrolidin-2-yl)-2,3,4,9-tetrahydro-1H-β-carboline

### Step A: (3R)-3-[4-Phenyl-1H-imidazol-2-yl]-1-((2S)-1-(tert-butyloxycarbonyl)-pyrrolidin-2-yl)-2,3,4,9-tetrahydro-1H-β-carboline

To a suspension of tert-butyl (1R)-2-(1H-indol-3-yl)-1-(4-phenyl-1H-imidazol-2-yl)-1-ethylcarbamate (Intermediate 1) (75 mg, 0.186 mmol) in CH₂Cl₂ (4 mL) was added TFA (2 mL). The reaction was stirred at rt for 1 h and then concentrated. The resulting material was dissolved in CH₂Cl₂ (4 mL) and (2S)-1-(tert-butyloxycarbonyl)-pyrrolidine-2-carboxaldehyde (74.3 mg, 0.373 mmol) was added. The reaction was stirred overnight at rt. Half of the material was concentrated to afford a residue which was purified by HPLC on a C-18 reverse-phase column eluting with a gradient of water (0.1% TFA) and acetonitrile (0.1% TFA). The fractions containing the product were lyophilized to afford (3R)-3-[4-phenyl-1H-imidazol-2-yl]-1-((2S)-1-(tert-butyloxycarbonyl)-pyrrolidin-2-yl)-2,3,4,9-tetrahydro-1H-β-carboline as a solid. ¹H NMR (500 MHz, CD₃OD): δ 7.80 (m, 3H), 7.52 (m, 3H), 7.45 (m, 2H), 7.16 (t, 1H), 7.07 (t, 1H), 5.04 (m, 1H), 4.66, (dd, 1H), 4.24 (m, 1H), 3.56 (m, 2H), 3.40 (m, 1H), 3.23, (m, 1H), 2.15 (m, 2H), 1.94 (m, 1H), 1.75 (m, 1H), 1.54 (s, 9H). LC-MS: m/z 484.29 (M+H)⁺ (3.09 min).

### Step B: (3R)-3-[4-phenyl-1H-imidazol-2-yl]-1-((2S)-pyrrolidin-2-yl)-2,3,4,9-tetrahydro-1H-β-carboline

A suspension of (3R)-3-[4-phenyl-1H-imidazol-2-yl]-1-((2S)-1-(tert-butyloxycarbonyl)-pyrrolidin-2-yl)-2,3,4,9-tetrahydro-1H-β-carboline (0.093 mmol) from Step A was dissolved in CH₂Cl₂ (2 mL) and treated with TFA (2 mL). The reaction mixture was stirred at rt for 1 h and then concentrated to afford a residue which was purified by HPLC on a C-18 reverse-phase column eluting with a gradient of water (0.1% TFA) and acetonitrile (0.1% TFA). The fractions containing the product were lyophilized to afford (3R)-3-[4-phenyl-1H-imidazol-2-yl]-1-((2S)-pyrrolidin-2-yl)-2,3,4,9-tetrahydro-1H-β-carboline as a solid. ¹H NMR (600 MHz, CD₃OD): δ 7.76 (m, 2H), 7.74 (m, 1H), 7.49 (m, 2H), 7.46 (m, 1H), 7.40 (m, 1H), 7.37 (m, 1H), 7.15 (t, 1H), 7.04 (t, 1H), 4.79, (d, 1H), 4.59 (dd, 1H), 4.17 (m, 1H), 3.46 (m, 1H), 3.24 (m, 1H), 3.20, (m, 1H), 3.07, (m, 1H), 2.32, (m, 1H), 2.15 (m, 1H), 2.11 (m, 1H), 2.09 (m, 1H). LC-MS: m/z 384.29 (M+H)^{˜} (2.29 min).

### Example 15

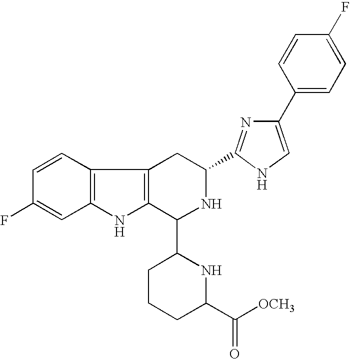

### (3R)-7-Fluoro-3-[4-(4-fluorophenyl)-1H-imidazol-2-yl]-1-(6-methoxycarbonyl-piperidin-2-yl)-2,3,4,9-tetrahydro-1H-β-carboline

### Step A: Piperidine-2,6-dicarboxylic acid, tert-butyl methyl diester

Piperidine-2,6-dicarboxylic acid, tert-butyl methyl diester was prepared according to the procedures described in J. Org. Chem. 46: 4914 (1981).

### Step B: 1-tert-Butyloxycarbonyl-6-hydroxymethyl-piperidine-2-carboxylic acid, methyl ester

To piperidine-2,6-dicarboxylic acid, tert-butyl methyl diester (2.3 g, 9.45 mmol) was added triethylsilane (3.77 mL, 23.63 mmol) followed by trifluoroacetic acid (14.57 mL, 189 mmol) at RT. The mixture was stirred at RT for 4 h. The reaction was concentrated, treated with MeOH (20 mL), triethylamine (3.95 mL, 28.4 mmol) followed by di-tert-butyl dicarbonate (2.68 g, 12.29 mmol). The reaction was stirred at RT for 48 h. Aqueous workup followed by concentration gave a residue which was treated with ice and 1N aqueous HCl and extracted with CH₂Cl₂. The combined organic layers were dried and concentrated to give a residue which was treated with tetrahydrofuran (10 mL) followed by BH₃ (1M solution in tetrahydrofuran) (18.91 mL, 18.91 mmol) at −78° C. The mixture was stirred overnight while warming to RT. The reaction was cooled to −78° C., treated with 20 mL water and warmed to RT. Aqueous workup followed by concentration gave a residue which was subjected to flash column chromatography on silica gel eluting with a gradient of 5% ethyl acetate in hexanes to 100% ethyl acetate affording 1-tert-butyloxycarbonyl-6-hydroxymethyl-piperidine-2-carboxylic acid, methyl ester. ¹H NMR (500 MHz, CDCl₃): δ 4.98 to 4.59 (broad, 1H), 4.36 (m, 1H), 3.78 (s, 3H), 3.56 (s, 2H), 2.41 (broad, 1H), 2.16 (s, 1H), 1.79 (m, 2H), 1.68 (m, 2H), 1.48 (m, 9H).

### Step C: 1-tert-Butyloxycarbonyl-piperidine-6-carboxaldehyde-1-carboxylic acid, methyl ester

To a solution of oxalyl chloride (2 M in CH₂Cl₂) (790 pt, 1.579 mmol) in CH₂Cl₂ (3 mL) was added dimethylsulfoxide (146 μL, 2.053 mmol) at −78° C. The mixture was stirred at −78° C. for 5 min and a solution of 1-tert-butyloxycarbonyl-6-hydroxymethyl-piperidine-2-carboxylic acid, methyl ester (332 mg, 1.215 mmol) in CH₂Cl₂ (2 mL) was added. The solution was stirred at −78° C. for 30 min, then triethylamine (1016 μL, 7.29 mmol) was added. The mixture was warmed to RT and diluted with ethyl acetate (20 mL) and water (20 mL). Extraction followed by concentration afforded 1-tert-butyloxycarbonyl-piperidine-6-carboxaldehyde-1-carboxylic acid, methyl ester, which was used in the next step without purification.

### Step D: (3R)-7-Fluoro-3-[4-(4-fluorophenyl)-1H-imidazol-2-yl]-1-(6-methoxycarbonyl-piperidin-2-yl)-2,3,4,9-tetrahydro-1H-β-carboline

To the faster-eluting enantiomer of tert-butyl 2-(6-fluoro-1H-indol-3-yl)-1-(4-(4-fluoropyridin-2-yl)-1H-imidazol-2-yl)-1-ethylcarbamate from Intermediate 6, Step G (300 mg, 0.684 mmol) was added CH₂Cl₂ (3 mL) followed by TFA (3 mL). The mixture was stirred at RT for 1 h. The reaction was concentrated and the residue was diluted with CH₂Cl₂ and concentrated again. The residue was treated with CH₂Cl₂ (3 mL) followed by tert-butyl 2-(6-fluoro-1H-indol-3-yl)-1-(4-(4-fluoropyridin-2-yl)-1H-imidazol-2-yl)-1-ethylcarbamate (330 mg, 1.215 mmol) in CH₂Cl₂ (3 mL). The mixture was stirred at RT overnight. The reaction was concentrated and then treated with MeOH (2 mL) followed by triethylamine (286 μL, 2.053 mmol) and di-tert-butyl dicarbonate (149 mg, 0.684 mmol) and stirred at RT for 2 h. The crude reaction product was recovered by preparative TLC and treated with TFA-CH₂Cl₂ to remove all the Boc groups. Aqueous work-up afforded a residue which was purified by preparative TLC eluting with 200:10:1 CH₂Cl₂/MeOH/NH₄OH to afford (3R)-7-fluoro-3-[4-(4-fluorophenyl)-1H-imidazol-2-yl]-1-(6-methoxycarbonyl-piperidin-2-yl)-2,3,4,9-tetrahydro-1H-β-carboline. LC-MS: m/z 492.27 (M+H)⁺ (2.46 min).

### Example 16

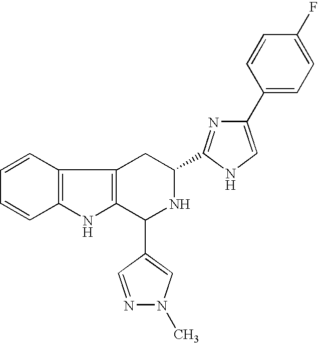

### (3R)-3-[4-(4-Fluorophenyl)-1H-imidazol-2-yl]-1-(1-methyl-1H-pyrazol-4-yl)-2,3,4,9-tetrahydro-1H-β-carboline

To a solution of (1R)-1-[4-(4-fluorophenyl)-1H-imidazol-2-yl]-2-(1H-indol-3-yl)ethanamine hydrochloride (200 mg, 0.56 mmol) [prepared by treatment of tert-butyl (1R)-2-(1H-indol-3-yl)-1-(4-(4-fluorophenyl)-1H-imidazol-2-yl)-1-ethylcarbamate (Intermediate 10) with hydrochloric acid] in MeOH (5 mL) was added 1-methyl-1H-pyrazole-4-carboxaldehyde (74 mg, 0.67 mmol) followed by a few drops of TFA. The mixture was stirred at rt overnight and then neutralized with 7 N ammonia in methanol (3 mL). The solvent was then removed under reduced pressure. The residue was purified by TLC chromatography to afford each individual diastereoisomer.

¹H NMR of the less polar product: (500 MHz, CD₃OD): δ 7.70 (m, 2H), 7.58 (s, 1H), 7.52 (s, 1H), 7.45 (d, 1H), 7.33 (s, 1H), 7.27 (d, 1H), 7.07 (m, 3H), 7.00 (m, 1H), 5.38 (s, 1H), 4.40 (dd, 1H), 3.85 (s, 3H), 3.17 (m, 2H). LC-MS: m/z 413 (M+1)⁺ at 2.48 min.

¹H NMR of the more polar product: (500 MHz, CD₃OD): δ 7.68 (m, 2H), 7.48 (d, 1H), 7.41 (s, 1H), 7.39 (s, 1H), 7.29 (s, 1H), 7.29 (d, 1H), 7.07 (m, 3H), 7.01 (m, 1H), 5.36 (s, 1H), 4.38 (dd, 1H), 3.80 (s, 3H), 3.18 (m, 2H). LC-MS: m/z 413 (M+1)⁺ at 2.56 min.

### Example 17

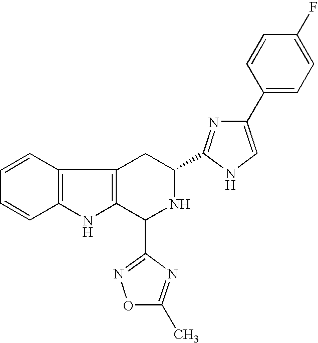

### (3R)-3-[4-(4-Fluorophenyl)-1H-imidazol-2-yl]-1-(5-methyl-1,2,4-oxadiazol-3-yl)-2,3,4,9-tetrahydro-1H-β-carboline

To a solution of (1R)-1-[4-(4-fluorophenyl)-1H-imidazol-2-yl]-2-(1H-indol-3-yl)ethanamine hydrochloride (200 mg, 0.56 mmol) [prepared by treatment of tert-butyl (1R)-2-(1H-indol-3-yl)-1-(4-(4-fluorophenyl)-1H-imidazol-2-yl)-1-ethylcarbamate (Intermediate 10) with hydrochloric acid] in MeOH (5 mL) was added 5-methyl-1,2,4-oxadiazole-3-carboxaldehyde (75 mg, 0.67 mmol) followed by a few drops of TFA. The mixture was stirred at rt overnight and then neutralized with 7 N ammonia in methanol (3 mL) before the solvent was removed under reduced pressure. The residue was purified by preparative TLC to give each diastereoisomer of (3R)-3-[4-(4-fluorophenyl)-1H-imidazol-2-yl]-1-(5-methyl-1,2,4-oxadiazol-3-yl)-2,3,4,9-tetrahydro-1H-β-carboline.

¹H NMR of the less polar product: (500 MHz, CD₃OD): δ 7.72 (m, 2H), 7.48 (d, 1H), 7.36 (s, 1H), 7.31 (d, 1H), 7.10 (m, 3H), 7.01 (t, 1H), 5.69 (s, 1H), 4.48 (dd, 1H), 3.23 (ddd, 1H), 3.13 (m, 1H), 2.61 (s, 3H). LC-MS: m/z 415 (M+1)⁺ at 2.65 min.

¹H NMR of the more polar product: (500 MHz, CD₃OD): δ 7.72 (m, 2H), 7.50 (d, 1H), 7.33 (s, 1H), 7.31 (d, 1H), 7.10 (m, 3H), 7.01 (t, 1H), 5.53 (s, 1H), 4.68 (dd, 1H), 3.25 (dd, 1H), 3.11 (ddd, 1H), 2.58 (s, 3H). LC-MS: m/z 415 (M+1)⁺ at 2.61 min.

The relative stereochemistry of the two products was determined by nuclear Overhauser effect (nOe) NMR spectroscopy. The less polar diastereoisomer afforded an nOe signal between the C-1 and C-3 hydrogens and the more polar product did not. Therefore, the less polar product was assigned as the cis-isomer and the more polar isomer as the trans-isomer.

### Example 18

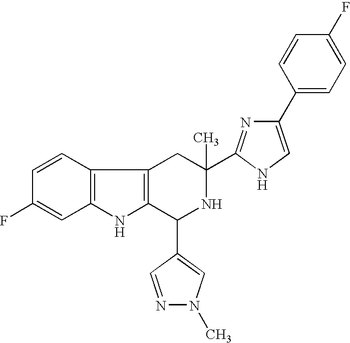

### 7-Fluoro-3-[4-(4-fluorophenyl)-1H-imidazol-2-yl]-3-methyl-1-(1-methyl-1H-pyrazol-4-yl)-2,3,4,9-tetrahydro-1H-β-carboline

To a solution of the faster-eluting enantiomer of tert-butyl 2-(6-fluoro-1H-indol-3-yl)-1-(4-(4-fluoropyridin-2-yl)-1H-imidazol-2-yl)-1-methyl-1-ethylcarbamate from Intermediate 7, Step H (100 mg, 0.22 mmol) in CH₂Cl₂ (5 mL) was added TFA (0.17 mL, 2.2 mmol) followed by N-methyl-4-formylpyrazole (24 mg, 0.67 mmol). The mixture was stirred at rt for 2 d, neutralized with 7 N ammonia in methanol, and the solvent removed under reduced pressure. The residue was purified by preparative TLC to give 7-fluoro-3-[4-(4-fluorophenyl)-1H-imidazol-2-yl]-3-methyl-1-(1-methyl-1H-pyrazol-4-yl)-2,3,4,9-tetrahydro-1H-β-carboline as a mixture of diastereoisomers in a 5:1 ratio. ¹H NMR of the major isomer: (500 MHz, CD₃OD): δ 7.67 (m, 2H), 7.52 (s, 1H), 7.48 (m, 1H), 7.43 (dd, 1H), 7.27 (s, 1H), 7.10 (m, 3H), 6.97 (dd, 1H), 6.79 (m, 1H), 5.36 (s, 1H), 3.81 (s, 3H), 3.36 (d, 1H), 3.10 (d, 1H), 1.68 (s, 3H). LC-MS: ink 445 (M+1)⁺ at 2.64 min.

### Example 19

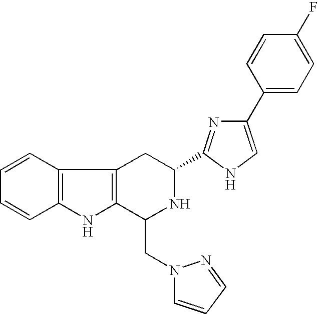

### (3R)-3-[4-(4-Fluorophenyl)-1H-imidazol-2-yl]-1-(1H-pyrazol-1-yl-methyl)-2,3,4,9-tetrahydro-1H-β-carboline

### Step A: 1-(2,2-Dimethoxyethyl)-1H-pyrazole

Pyrazole (749 mg, 11 mmol) was dissolved in DMF (5 mL) and was cooled to 0° C. To this solution was slowly added NaH (60% in mineral oil, 440 mg, 10 mmol). After the mixture was stirred at 0° C. for 10 min and at rt for 2 h, 1,1-dimethoxy-2-bromo-ethane (1.69 g, 10 mmol) was added. The mixture was stirred for 1 day, diluted with EtOAc, washed with water, and brine. The organic layer was dried and evaporated to give 1-(2,2-dimethoxyethyl)-1H-pyrazole as a colorless oil. ¹H NMR (500 MHz, CDCl₃): δ 7.51 (d, 1H), 7.44 (d, 1H), 6.25 (t, 1), 4.65 (t, 1H), 4.22 (d, 2H), 3.36 (s; 6H).

### Step B: (3R)-3-[4-(4-Fluorophenyl)-1H-imidazol-2-yl]-1-(1H-pyrazol-1-yl-methyl)-2,3,4,9-tetrahydro-1H-β-carboline

To a solution of (1R)-1-[4-(4-fluorophenyl)-1H-imidazol-2-yl]-2-(1H-indol-3-yl)ethanamine hydrochloride (50 mg, 0.14 mmol) [prepared by treatment of tert-butyl (1R)-2-(1H-indol-3-yl)-1-(4-(4-fluorophenyl)-1H-imidazol-2-yl)-1-ethylcarbamate (Intermediate 10) with hydrochloric acid] in CH₂Cl₂ (1 mL) was added TFA (50 μL) followed by 1-(2,2-dimethoxyethyl)-1H-pyrazole (33 mg, 0.21 mmol). The mixture was stirred at rt overnight, diluted with EtOAc and washed with saturated NaHCO₃ and brine. The organic layer was separated, dried and evaporated to give a crude residue that was purified by preparative TLC to give (3R)-3-[4-(4-fluorophenyl)-1H-imidazol-2-yl]-1-(1H-pyrazol-1-yl-methyl)-2,3,4,9-tetrahydro-1H-β-carboline. ¹H NMR (500 MHz, DMSO-d₆): δ 11.2 (s, 1H), 7.82 (m, 2H), 7.77 (d, 1H), 7.64 (s, 1H), 7.54 (s, 1H), 7.46 (d, 1H), 7.40 (d, 1H), 7.22 (m, 2H), 7.12 (t, 1H), 7.02 (t, 1H), 6.27 (s, 1H), 4.95 (d, 2H), 4.58 (m, 1H), 4.42 (br, 1H), 3.19 (m, 1H), 3.06 (m, 1H). LC-MS: m/z 413 (M+1)⁺ at 2.71 min.

### Example 20

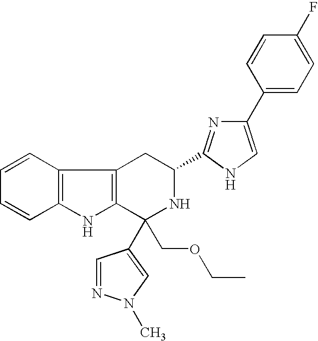

### (3R)-[4-(4-Fluorophenyl)-1H-imidazol-2-yl]-1-(ethoxymethyl)-1-(1-methyl-1H-pyrazol-4-yl)-2,3,4,9-tetrahydro-1H-β-carboline

(1R)-1-[4-(4-fluorophenyl)-1H-imidazol-2-yl]-2-(1H-indol-3-yl)ethanamine hydrochloride (450 mg, 1.261 mmol) [prepared by treatment of tert-butyl (1R)-2-(1H-indol-3-yl)-1-(4-(4-fluorophenyl)-1H-imidazol-2-yl)-1-ethylcarbamate (Intermediate 10) with hydrochloric acid] was treated with pyridine (5 mL) followed by 2-ethoxy-1-(1-methyl-pyrazol-4-yl)-ethanone (Intermediate 20) (297 mg, 1.766 mmol). The mixture was heated under N₂ (oil bath 70° C.) for 2.5 d followed by additional heating (oil bath 80° C.) for 24 h. The reaction mixture was concentrated and the residue was purified by preparative TLC eluting with 20:1 CH₂Cl₂: MeOH to give (3R)-[4-(4-fluorophenyl)-1H-imidazol-2-yl]-1-(ethoxy-methyl)-1-(1-methyl-1H-pyrazol-4-yl)-2,3,4,9-tetrahydro-1H-β-carboline as a mixture of diastereoisomers. These isomers were separated by preparative chiral HPLC to afford the individual diastereoisomers. The isomers were characterized by an analytical chiral AD column eluting with 20% IPA in heptane.

(3R)-[4-(4-Fluorophenyl)-1H-imidazol-2-yl]-1-(ethoxymethyl)-(1R)-(1-methyl-1H-pyrazol-4-yl)-2,3,4,9-tetrahydro-1H-β-carboline (faster eluting isomer: retention time 19.78 min): ¹H NMR (500 MHz, MeOH-d₄): δ 7.72 (m, 2H), 7.57 (s, 1H), 7.53 (s, 1H), 7.49 (d, 1H), 7.33 (m, 2H), 7.11 (m, 3H), 7.03 (t, 1H), 4.73 (dd, 1H), 4.06 (s, 2H), 3.84 (s, 3H), 3.58 (m, 2H), 3.20 (dd, 1H), 3.05 (dd, 1H), 1.21 (t, 3H). LC-MS: m/z 471.1 (M+H)⁺ (2.62 min).

(3R)-[4-(4-Fluorophenyl)-1H-imidazol-2-yl]-1-(ethoxymethyl)-(1S)-(1-methyl-1H-pyrazol-4-yl)-2,3,4,9-tetrahydro-1H-β-carboline (slower eluting isomer: retention time 25.79 min): ¹H NMR (500 MHz, MeOH-d₄): δ 7.72 (m, 2H), 7.48 (d, 1H), 7.39 (m, 3H), 7.34 (s, 1H), 7.12 (m, 3H), 7.04 (t, 1H), 4.29 (dd, 1H), 4.04 (d, 1H), 3.93 (d, 1H), 3.81 (s, 3H), 3.57 (m, 2H), 3.13 (m, 2H), 1.17 (t, 3H). LC-MS: m/z 471.1 (M+H)⁺ (2.67 min).

The relative stereochemistry of the two diastereoisomers was determined by nuclear Overhauser effect (nOe) NMR spectroscopy. The slower eluting diastereoisomer afforded a nOe signal between the C-3 and C-5 hydrogens on the C-1 pyrazole and the C-3 hydrogen on the β-carboline and the faster eluting product did not. Therefore, the diastereoisomer that eluted first from the preparative chiral HPLC purification was assigned as the cis-isomer (imidazole and pyrazole are cis) and the slower eluting isomer as the trans-isomer.

### Example 21

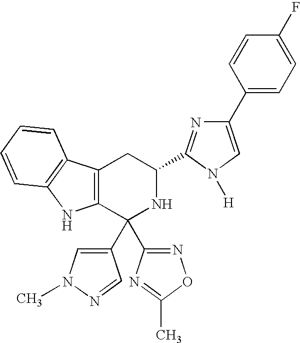

### (3R)-[4-(4-Fluorophenyl)-1H-imidazol-2-yl]-1-(5-methyl-1,2,4-oxadiazol-3-yl)-1-(1-methyl-1H-pyrazol-4-yl)-2,3,4,9-tetrahydro-1H-β-carboline

(1R)-1-[4-(4-Fluorophenyl)-1H-imidazol-2-yl]-2-(1H-indol-3-yl)ethanamine hydrochloride (370 mg, 1.037 mmol) [prepared by treatment of tert-butyl (1R)-2-(1H-indol-3-yl)-1-(4-(4-fluorophenyl)-1H-imidazol-2-yl)-1-ethylcarbamate with hydrochloric acid] was treated with pyridine (4 mL) followed by reaction with 1-methyl-pyrazol-4-yl 5-methyl-1,2,4-triazol-3-yl ketone (Intermediate 22) (219 mg, 1.141 mmol). The reaction was heated under N₂ (oil bath 70° C.) for 48 h followed by additional heating (oil bath 85° C.) for 3 d. The reaction mixture was concentrated and azeotroped with toluene. The residue was purified with preparative TLC eluting with 10% MeOH in CH₂Cl₂ to give (3R)-[4-(4-fluorophenyl)-1H-imidazol-2-yl]-1-(5-methyl-1,2,4-oxadiazol-3-yl)-1-(1-methyl-pyrazol-4-yl)-2,3,4,9-tetrahydro-1H-β-carboline as a mixture of diastereoisomers which were separated by chiral HPLC. The isomers were characterized by an analytical chiral AD column eluting with 20% IPA in heptane. (3R)-[4-(4-Fluorophenyl)-1H-imidazol-2-yl]-1-(5-methyl-1,2,4-oxadiazol-3-yl)-(1R)-(1-methyl-pyrazol-4-yl)-2,3,4,9-tetrahydro-1H-β-carboline (faster eluting isomer: retention time 18.13 min): ¹H NMR (500 MHz, MeOH-d₄): δ 7.74 (m, 2H), 7.65 (s, 1H), 7.52 (m, 2H), 7.37 (m, 2H), 7.13 (m, 3H), 7.04 (s, 1H), 4.47 (dd, 1H), 3.87 (s, 3H), 3.24 (dd, 1H), 3.16 (dd, 1H), 2.63 (s, 3H). LC-MS: m/z 495.3 (M+H)⁺ (2.56 min).

(3R)-[4-(4-Fluorophenyl)-1H-imidazol-2-yl]-1-(5-methyl-1,2,4-oxadiazol-3-yl)-(1S)-(1-methyl-pyrazol-4-yl)-2,3,4,9-tetrahydro-1H-β-carboline (slower eluting isomer: retention time 24.62 min): ¹H NMR (500 MHz, MeOH-d₄): δ 7.73 (m, 2H), 7.54 (d, 1H), 7.48 (s, 1H), 7.43 (s, 1H), 7.40 (d, 1H), 7.36 (brs, 1H), 7.13 (m, 3H), 7.06 (t, 1H), 4.40 (dd, 1H), 3.84 (s, 3H), 3.26 (dd, 1H), 3.16 (dd, 1H), 2.63 (s, 3H). LC-MS: m/z 495.3 (M+H)⁺ (2.61 min).

The relative stereochemistry of the two diastereoisomers was determined by nuclear Overhauser effect (nOe) NMR spectroscopy. The slower eluting diastereisoomer afforded an nOe signal between the C-3 and C-5 hydrogens on the C-1 pyrazole and the C-3 hydrogen on the β-carboline and the faster eluting product did not. Therefore, the diastereoisomer that eluted first from the preparative chiral HPLC purification was assigned as the cis-isomer (imidazole and pyrazole are cis) and the slower eluting isomer as the trans-isomer.

The Examples shown in Table 2 were prepared from the appropriately substituted tert-butyl 2-(1H-indol-3-yl)-1-(4-aryl-1H-imidazol-2-yl)-1-ethylcarbamate derivative and a substituted heterocyclic or heteroaryl carboxaldehyde according to the methods described in Examples 1-21.

TABLE 2

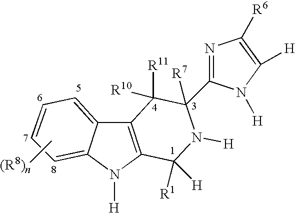

| | | | | | | LC-MS |
|---|---|---|---|---|---|---|
| | | | | R¹⁰, | | m/z |
| Ex. Number | R¹ | R⁶ | R⁷ | R¹¹ | R⁸ | (M + H)⁺ |
| 22 | oxazol-4-yl | phenyl | H | H, H | H | 382.2 |
| 23 | pyridazin-3-yl | phenyl | H | H, H | H | 393.1 |
| 24 | pyrazin-2-yl | phenyl | H | H, H | H | 393.2 |
| 25 | thiazol-4-yl | phenyl | H | H, H | H | 398.2 |
| 26 | thiazol-5-yl | phenyl | H | H, H | H | 398.1 |
| 27 | pyrazol-4-yl | 4-F-phenyl | CH₃ | H, H | H | 413.0 |
| 28 | piperidin-4-yl | 4-F-phenyl | H | H, H | H | 416.1 |
| 29 | 1-ethyl-pyrazol- | phenyl | CH₃ | H, H | H | 423.0 |
| | 4-yl | | | | | |
| 30 | 1-methyl-pyrazol- | 4-F-phenyl | CH₃ | H, H | H | 427.0 |
| | 4-yl | | | | | |
| 31 | 5-chloro-pyridazin- | phenyl | H | H, H | H | 427.1 |
| | 2-yl | | | | | |
| 32 | pyrimidin-2-yl | 4-F-phenyl | H | H, H | 7-F | 429.0 |
| 33 | 5-methyl-1,2,4- | 4-F-phenyl | CH₃ | H, H | H | 429.0 |
| | oxadiazol-3-yl | | | | | |
| 34 | 2-methyl-thiazol- | 4-F-phenyl | H | H, H | H | 430.0 |
| | 5-yl | | | | | |
| 35 | 1-methyl-pyrazol- | 4-F-phenyl | H | H, H | 7-F | 431.0 |
| | 4-yl | | | | | |
| 36 | benzimidazol-2-yl | phenyl | H | H, H | H | 431.2 |
| 37 | 1-isopropyl-pyrazol- | 4-F-phenyl | H | H, H | H | 441.0 |
| | 4-yl | | | | | |
| 38 | 1-ethyl-pyrazol- | 4-F-phenyl | CH₃ | H, H | H | 441.0 |
| | 4-yl | | | | | |
| 39 | 1,5-dimethyl- | 4-F-phenyl | CH₃ | H, H | H | 441.1 |
| | pyrazol-3-yl | | | | | |
| 40 | 1,2-dimethyl- | 4-F-phenyl | CH₃ | H, H | H | 441.1 |
| | imidazol-5-yl | | | | | |
| 41 | 3-amino-1-methyl- | 4-F-phenyl | CH₃ | H, H | H | 441.1 |
| | pyrazol-4-yl | | | | | |
| 42 | 2,4-dimethyl- | 4-F-phenyl | H | H, H | H | 444.0 |
| | thiazol-5-yl | | | | | |
| 43 | 2-methyl-thiazol- | 4-F-phenyl | H | H, H | 7-F | 448.1 |
| | 5-yl | | | | | |
| 44 | 1-acetyl-piperidin- | 4-F-phenyl | H | H, H | H | 458.0 |
| | 4-yl | | | | | |
| 45 | 2-methoxy-pyrimidin- | 4-F-phenyl | H | H, H | 7-F | 459.0 |
| | 5-yl | | | | | |
| 46 | 1-isopropyl-pyrazol- | 4-F-phenyl | H | H, H | 7-F | 459.0 |
| | 4-yl (isomer A) | | | | | |
| 47 | 1-isopropyl-pyrazol- | 4-F-phenyl | H | H, H | 7-F | 459.0 |
| | 4-yl (isomer B) | | | | | |
| 48 | 2-methyl-thiazol- | 4-F-phenyl | CH₃ | H, H | 7-F | 462.0 |
| | 5-yl | | | | | |
| 49 | 3-cyclopropyl-1-methyl- | 4-F-phenyl | H | H, H | 7-F | 471.0 |
| | pyrazol-4-yl | | | | | |
| 50 | 1-isopropyl-pyrazol- | 4-F-phenyl | CH₃ | H, H | 7-F | 473.0 |
| | 4-yl | | | | | |
| 51 | 2-diethylamino-thiazol- | 4-F-phenyl | H | H, H | 7-F | 505.0 |
| | 5-yl | | | | | |
| 52 | 1-methyl-3-phenyl- | 4-F-phenyl | H | H, H | 7-F | 507.0 |
| | pyrazol-4-yl | | | | | |
| 53 | 4-phenyl-thiazol-2-yl | 4-F-phenyl | H | H, H | 7-F | 509.9 |
| 54 | 2-phenyl-thiazol-5-yl | 4-F-phenyl | H | H, H | 7-F | 509.9 |
| 55 | 1-(4-fluoro-phenyl)- | 4-F-phenyl | H | H, H | 7-F | 511.0 |
| | pyrazol-4-yl | | | | | |
| 56 | 1-methyl-sulfonyl- | 4-F-phenyl | H | H, H | 7-F | 512.1 |
| | piperidin-3 -yl | | | | | |
| | (isomer A) | | | | | |
| 57 | 1-methyl-sulfonyl- | 4-F-phenyl | H | H, H | 7-F | 512.1 |
| | piperidin-3 -yl | | | | | |
| | (isomer B) | | | | | |
| 58 | 1-tert-butyloxycarbonyl- | 4-F-phenyl | H | H, H | H | 516.0 |
| | piperidin-4-yl | | | | | |
| 59 | 1-(benzyloxy-carbonyl)- | 4-F-phenyl | H | H, H | H | 550.1 |
| | piperidin-4-yl | | | | | |
| 60 | pyrimidin-5-yl | 4-F-phenyl | H | H, H | H | 411.2 |
| 61 | 1-methyl-imidazol-4-yl | 4-F-phenyl | H | H, H | H | 413.1 |
| 62 | 2-methyl-imidazol-4-yl | 4-F-phenyl | H | H, H | H | 413.1 |
| 63 | 5-methyl-isoxazol-3-yl | 4-F-phenyl | H | H, H | H | 414.3 |
| 64 | 3-methyl-1,2,4- | 4-F-phenyl | H | H, H | H | 415.1 |
| | oxadiazol-5-yl | | | | | |
| 65 | piperidin-4-yl | Phenyl | H | H, H | H | 415.1 |
| 66 | 1-acetyl-piperidin-4-yl | Phenyl | H | H, H | H | 440.2 |
| 67 | 1-(N-methyl- | Phenyl | H | H, H | H | 455.1 |
| | carbamoyl)-piperidin- | | | | | |
| | 4-yl | | | | | |
| 68 | 1-(methoxy-carbonyl)- | Phenyl | H | H, H | H | 456.2 |
| | piperidin-4-yl | | | | | |
| 69 | 1-(methyl-sulfonyl)- | Phenyl | H | H, H | H | 467.2 |
| | piperidin-4-yl | | | | | |
| 70 | tetrahydropyran-4-yl | Phenyl | H | H, H | H | 399.3 |
| | (1R isomer) | | | | | |
| 71 | 1-succinyl-piperidin- | Phenyl | H | H, H | H | 498.1 |
| | 4-yl | | | | | |
| 72 | 1-(tert-butyloxy- | Phenyl | H | H, H | H | 498.2 |
| | carbonyl)-piperidin-4-yl | | | | | |
| 73 | 1-(2-carboxy-benzoyl)- | Phenyl | H | H, H | H | 546.1 |
| | piperidin-4-yl | | | | | |
| 74 | tetrahydropyran-4-yl | Pyridin-2-yl | H | H, H | H | 400.0 |
| 75 | tetrahydropyran-4-yl | Pyridin-2-yl | H | H, H | 7-F | 418.0 |
| 76 | tetrahydropyran-4-yl | 5-F-pyridin- | H | H, H | H | 418.0 |
| | | 2-yl | | | | |
| 77 | 1-methyl-pyrazol-4-yl | 5-F-pyridin- | H | H, H | 7-F | 432.0 |
| | (1R,3R isomer) | 2-yl | | | | |
| 78 | 1-methyl-pyrazol-4-yl | 5-F-pyridin- | H | H, H | 7-F | 432.0 |
| | (1S,3R isomer) | 2-yl | | | | |
| 79 | tetrahydropyran-4-yl | 5-F-pyridin- | CH₃ | H, H | H | 432.0 |
| | | 2-yl | | | | |
| 80 | Tetrahydropyran-4-yl | 5-Cl-pyridin- | H | H, H | H | 434.0 |
| | | 2-yl | | | | |
| 81 | Tetrahydropyran-4-yl | 5-F-pyridin- | H | H, H | 7-F | 436.0 |
| | | 2-yl | | | | |
| 82 | 1-methyl-pyrazol-4-yl | 2,1,3- | H | H, H | H | 437.0 |
| | | benzoxadiazol- | | | | |
| | | 5-yl | | | | |
| 83 | 1-methyl-pyrazol-3-yl | 2,1,3- | H | H, H | H | 437.0 |
| | | benzoxadiazol- | | | | |
| | | 5-yl | | | | |
| 84 | 5-methyl-1,2,4- | 2,1,3- | H | H, H | H | 439.0 |
| | oxadiazol-3-yl | benzoxadizaol- | | | | |
| | | 5-yl | | | | |
| 85 | Tetrahydropyran-4-yl | 2,1,3- | H | H, H | H | 441.0 |
| | | benzoxadiazol- | | | | |
| | | 5-yl | | | | |
| 86 | 5-methyl-1,2,4- | 5-F-pyridin- | CH₃ | H, H | 7-F | 478.3 |
| | oxadiazol-3-yl | 2-yl | | | | |
| 87 | 1,2,3-thiadiazol-4-yl | 4-F-phenyl | H | H, H | H | 416.9 |
| 88 | 1-methyl-pyrazol-4-yl | 4-F-phenyl | H | CH₃, H | H | 427.1 |
| 89 | 5-methyl-1,2,4- | 5-F-phenyl | CH₃ | H, H | 7-F | 433.1 |
| | oxadiazol-3-yl | | | | | |
| 90 | 1-isopropyl-pyrazol-4-yl | 4-F-phenyl | CH₃ | H, H | H | 455.2 |
| 91 | 6-carboxy-piperidin-2-yl | 4-F-phenyl | H | H, H | 7-F | 478.2 |
| 92 | 1-(2-methoxyethyl)- | 4-F-phenyl | H | H, H | 7-F | 492.0 |
| | piperidin-4-yl | | | | | |
| 93 | 1-methyl-pyrazol-4-yl | 4-F-phenyl | H | CH₃, H | H | 427.1 |
| 94 | 1-methyl-pyrazol-4-yl | Phenyl | H | H, H | 7-CN | 420.1 |
| 95 | 5-methyl-1,2,4- | Pyridin-2-yl | H | H, H | 7-Cl | 432.0 |
| | oxadiazol-3-yl | | | | | |
| 96 | 1-methyl-pyrazol-4-yl | Phenyl | H | H, H | 7-CN, | 438.0 |
| | | | | | 6-F | |
| 97 | 1-methyl-pyrazol-4-yl | 4-F-phenyl | CH₃ | H, H | 7-CN | 452.4 |
| | (1R isomer) | | | | | |
| 98 | 1-methyl-pyrazol-4-yl | 4-F-phenyl | CH₃ | H, H | 7-CN | 452.4 |
| | (1S isomer) | | | | | |
| 99 | 1-methyl-pyrazol-4-yl | 4-F-phenyl | CH₃ | H, H | 7-Cl | 461.0 |
| | (1S isomer) | | | | | |
| 100 | 1-methyl-pyrazol-4-yl | 4-F-phenyl | CH₃ | H, H | 7-Cl | 461.0 |
| | (1R isomer) | | | | | |
| 101 | 1-methyl-pyrazol-3-yl | 4-F-phenyl | CH₃ | H, H | 7-Cl | 461.1 |
| | (1R isomer) | | | | | |
| 102 | 1-methyl-pyrazol-3-yl | 4-F-phenyl | CH₃ | H, H | 7-Cl | 461.2 |
| | (1S isomer) | | | | | |
| 103 | 1-methyl-1,2,4- | 4-F-phenyl | CH₃ | H, H | 7-Cl | 463.2 |
| | oxadiazol-3-yl | | | | | |
| | (1R isomer) | | | | | |
| 104 | 1-methyl-1,2,4- | 4-F-phenyl | CH₃ | H, H | 7-Cl | 463.2 |
| | oxadiazol-3-yl | | | | | |
| | (1S isomer) | | | | | |
| 105 | 1-methyl-pyrazol-3-yl | phenyl | H | H, H | 7-Br | 474.9 |
| | (1S isomer) | | | | | |
| 106 | 1-methyl-pyrazol-3-yl | phenyl | H | H, H | 7-Br | 474.9 |
| | (1R isomer) | | | | | |
| 107 | 1-ethyl-pyrazol-3-yl | 4-F-phenyl | CH₃ | H, H | 7-Cl | 475.2 |
| | (1S isomer) | | | | | |
| 108 | 1-methyl-1,2,4- | phenyl | H | H, H | 7-Br | 476.9 |
| | oxadiazol-3-yl | | | | | |
| | (1S isomer) | | | | | |
| 109 | 1-methyl-1,2,4- | phenyl | H | H, H | 7-Br | 477.1 |
| | oxadiazol-3-yl | | | | | |
| | (1R isomer) | | | | | |
| 110 | 1-methyl-pyrazol-3- | 4-F-phenyl | CH₃ | H, H | 7-Br | 507.0 |
| | yl (1S isomer) | | | | | |
| 111 | 1-methyl-pyrazol-3-yl | 4-F-phenyl | CH₃ | H, H | 7-Br | 507.0 |
| | (1R isomer) | | | | | |
| 112 | 1-methyl-1,2,4- | 4-F-phenyl | H | H, H | 7-Br | 509.0 |
| | oxadiazol-3-yl | | | | | |
| | (1R isomer) | | | | | |
| 113 | 1-methyl-1,2,4- | 4-F-phenyl | H | H, H | 7-Br | 508.9 |
| | oxadiazol-3-yl | | | | | |
| | (1S isomer) | | | | | |
| 114 | Tetrahydropyran-4-yl | 4-F-phenyl | H | H, H | 6-(6-F- | 512.1 |
| | | | | | pyrid- | |
| | | | | | 3-yl | |
| 115 | 4-methyl-imidazol-2-yl | 4-F-phenyl | H | H, H | H | 413.0 |
| 116 | 1-methyl-pyrazol-4-yl | 4-F-phenyl | CH₃ | H, H | 8-F | 445.0 |
| 117 | 1-methyl-1,2,4- | Pyridin-2-yl | CH₃ | H, H | 7-Cl | 446.0 |
| | oxadiazol-3-yl | | | | | |
| | (1S isomer) | | | | | |
| 118 | 1-methyl-1,2,4- | 4-F-phenyl | CH₃ | H, H | 8-F | 447.0 |
| | oxadiazol-3-yl | | | | | |
| | (1S isomer) | | | | | |
| 119 | Oxazol-4-yl | 4-F-phenyl | H | H, H | H | 400.2 |
| 120 | 2-methyl-oxazol-4-yl | 4-F-phenyl | H | H, H | H | 414.2 |
| | (isomer A) | | | | | |
| 121 | 2-methyl-oxazol-4-yl | 4-F-phenyl | H | H, H | H | 414.3 |
| | (isomer B) | | | | | |
| 122 | 2,5-dimethyl-oxazol-4- | 4-F-phenyl | H | H, H | H | 428.3 |
| | yl (isomer A) | | | | | |
| 123 | 2,5-dimethyl-oxazol-4- | 4-F-phenyl | H | H, H | H | 428.3 |
| | yl (isomer B) | | | | | |
| 124 | Indazol-6-yl | 4-F-phenyl | H | H, H | H | 449.0 |
| 125 | Oxazol-2-yl | 4-F-phenyl | H | H, H | H | 400.0 |
| 126 | 1,2,4-triazol-3-yl | 4-F-phenyl | H | H, H | H | 400.0 |
| 127 | 1-methyl-1,2,4-triazol- | 4-F-phenyl | H | H, H | H | 414.1 |
| | 3-yl | | | | | |
| 128 | 1,5-dimethyl-pyrazol-3- | 4-F-phenyl | H | H, H | H | 427.1 |
| | yl (isomer_A) | | | | | |
| 129 | 1,5-dimethyl-pyrazol-4- | 4-F-phenyl | H | H, H | H | 427.1 |
| | yl (isomer B) | | | | | |
| 130 | 1-methyl-pyrazol-3-yl | phenyl | H | H, H | H | 395.2 |
| | (isomer A) | | | | | |
| 131 | 1-methyl-pyrazol-3-yl | phenyl | H | H, H | H | 395.2 |
| | (isomer A) | | | | | |
| 132 | 5-methyl-1,2,4- | phenyl | H | H, H | H | 397.2 |
| | oxadiazol-3-yl | | | | | |
| 133 | pyrazol-3-yl | 4-F-phenyl | H | H, H | H | 399.4 |
| | (isomer A) | | | | | |
| 134 | pyrazol-3-yl | 4-F-phenyl | H | H, H | H | 399.4 |
| | (isomer B) | | | | | |
| 135 | pyrazol-4-yl | 4-F-phenyl | H | H, H | H | 399.4 |
| 136 | 1,2,3-triazol-4-yl | 4-F-phenyl | H | H, H | H | 400.1 |
| 137 | 1,2,4-oxadiazol-3-yl | 4-F-phenyl | H | H, H | H | 401.1 |
| 138 | 2-methyl-pyrazol-3-yl | 4-F-phenyl | H | H, H | H | 413.5 |
| 139 | 1-methyl-pyrazol-3-yl | 4-F-phenyl | H | H, H | H | 413.2 |
| 140 | 5-methyl-pyrazol-3-yl | 4-F-phenyl | H | H, H | H | 413.1 |
| 141 | 1-ethyl-pyrazol-4-yl | 4-F-phenyl | H | H, H | H | 427.1 |
| | (isomer A) | | | | | |
| 142 | 1-ethyl-pyrazol-4-yl | 4-F-phenyl | H | H, H | H | 427.1 |
| | (isomer B) | | | | | |
| 143 | 1,5-dimethyl-pyrazol-4-yl | 4-F-phenyl | H | H, H | H | 427.1 |
| 144 | 2,5-dimethyl-pyrazol-3-yl | 4-F-phenyl | H | H, H | H | 427.2 |
| | (isomer A) | | | | | |
| 145 | 2,5-dimethyl-pyrazol-3- | 4-F-phenyl | H | H, H | H | 427.2 |
| | (isomer B) | | | | | |
| 146 | 1-methyl-pyrazol-4-yl | 4-F-phenyl | CH₃ | H, H | 7-F | 445.1 |
| | (isomer A) | | | | | |
| 147 | 1-methyl-pyrazol-4-yl | 4-F-phenyl | CH₃ | H, H | 7-F | 445.1 |
| 148 | 5-methyl-1,2,4- | 4-F-phenyl | CH₃ | H, H | 7-F | 447.1 |
| | oxadiazol-3-yl | | | | | |
| 149 | 4-chloro-1-methyl- | 4-F-phenyl | H | H, H | H | 447.1 |
| | pyrazol-3-yl | | | | | |
| 150 | Pyrazolo[2,3-a]pyrid- | 4-F-phenyl | H | H, H | H | 449.1 |
| | 3-yl | | | | | |
| 151 | Pyrazolo[2,3-a]pyrid- | 4-F-phenyl | H | H, H | H | 449.1 |
| | 7-yl | | | | | |
| 152 | [1H]-2-guinolon-3-yl | 4-F-phenyl | H | H, H | H | 476.1 |
| 153 | 1-(tert-butyl 2-methyl-2- | 4-F-phenyl | H | H, H | H | 541.2 |
| | propanoate)-pyrazol-4-yl | | | | | |
| 154 | 2,3,4,5-tetrahydro-2- | 4-F-phenyl | H | H, H | H | 443 |
| | methyl-3-pyridazinon-6-yl | | | | | |
| 155 | 1,4,4-trimethyl-4,5- | 4-F-phenyl | H | H, H | H | 457 |
| | dihydro-5-pyrazolon-3-yl | | | | | |
| 156 | 2-methyl-thiazol-5-yl | 4-F-phenyl | CH₃ | H, H | H | 444 |
| 157 | 2-amino-thiazol-5-yl | 4-F-phenyl | H | H, H | H | 431 |
| 158 | Tetrahydropyran-4-yl | 4-F-phenyl | H | CH₃, H | 7-F | 449.1 |
| 159 | 2-isopropyl-thiazol-4-yl | 4-F-phenyl | H | H, H | H | 458 |
| 160 | 5-methyl-1,2,4- | 4-F-phenyl | H | CH₃, H | 7-F | 447.1 |
| | oxadiazol-3-yl | | | | | |
| 161 | 4-methyl-thiazol-2-yl | 4-F-phenyl | H | H, H | H | 430.1 |
| 162 | 2,1,3-benzoxadiazol-5-yl | 4-F-phenyl | H | H, H | H | 451.1 |
| 163 | 2-oxo-tetrahydrofuran- | 4-F-phenyl | H | H, H | H | 417 |
| | 4-yl | | | | | |
| 164 | 5-cyclopropyl-1,2,4- | 4-F-phenyl | H | H, H | H | 441 |
| | oxadiazol-3-yl | | | | | |
| 165 | 5-ethyl-1,2,4-oxadiazol- | 4-F-phenyl | H | H, H | H | 429 |
| | 3-yl | | | | | |
| 166 | 5-(1-hydroxy-1-methyl- | 4-F-phenyl | H | H, H | H | 459 |
| | ethyl)-1,2,4-oxadiazol-3-yl | | | | | |
| 167 | 5-uracilyl | 4-F-phenyl | H | H, H | H | 443.3 |
| 168 | 1-methyl-pyrazol-4-yl | 5-F-pyridin- | CH₃ | H, H | 7-Cl | 462 |
| | | 2-yl | | | | |
| 169 | 5-methyl-1,2,4- | 5-F-pyridin- | CH₃ | H, H | 7-Cl | 464 |
| | oxadiazol-3-yl | 2-yl | | | | |
| 170 | 2-methoxy-carbonyl-2- | 4-F-phenyl | H | H, H | H | 489 |
| | methyl-tetrahydropyran- | | | | | |
| | 4-yl | | | | | |
| 171 | 1,2,3-thiadiazol-4-yl | 4-F-phenyl | H | H, H | H | 417 |
| 172 | Isothiazol-4-yl | 4-F-phenyl | H | H, H | H | 416 |
| 173 | 2-carboxy-2-methyl- | 4-F-phenyl | H | H, H | H | 475.1 |
| | tetrahydropyran-4-yl | | | | | |
| 174 | 1-isopropyl-pyrazol-4-yl | 4-F-phenyl | CH₃ | H, H | 7-Cl | 489 |
| | (isomer A) | | | | | |
| 175 | 1-isopropyl-pyrazol-4-yl | 4-F-phenyl | CH₃ | H, H | 7-Cl | 489 |
| | (isomer B) | | | | | |
| 176 | 5-methyl-1,2,4- | 4-F-phenyl | CH₃ | H, H | 5-CN | 453.9 |
| | oxadiazol-3-yl | | | | | |
| 177 | 5-dimethyl-amino-1,2,4- | 4-F-phenyl | H | H, H | H | 444.35 |
| | oxadiazol-3-yl | | | | | |
| 178 | 5-(4-morpholinyl)- | 4-F-phenyl | H | H, H | H | 486.25 |
| | 1,2,4-oxadiazol-3-yl | | | | | |
| 179 | 5-(1-pyrrolidinyl)-1,2,4- | 4-F-phenyl | H | H, H | H | 470.2 |
| | oxadiazol-3-yl | | | | | |
| 180 | 5-methyl-1,2,4- | 4-F-phenyl | H | H, H | 6-I | 541 |
| | oxadiazol-3-yl | | | | | |
| 181 | 1,2,4-triazol-5-on-3-yl | 4-F-phenyl | H | H, H | H | 416 |
| 182 | 2-methyl-1,2,3-triazol- | 4-F-phenyl | H | H, H | H | 414 |
| | 4-yl | | | | | |
| 183 | 1-methyl-1,2,3-triazol- | 4-F-phenyl | H | H, H | H | 414 |
| | 4-yl (isomer A) | | | | | |
| 184 | 1-methyl-1,2,3-triazol- | 4-F-phenyl | H | H, H | H | 414 |
| | 4-yl (isomer B) | | | | | |
| 185 | Tetrahydropyran-4-yl | 4-methyl- | H | H, H | H | 419 |
| | | thien-2-yl | | | | |
| 186 | Tetrahydropyran-4-yl | Tetrazolo- | H | H, H | H | 441 |
| | | [1,5-a] | | | | |
| | | pyrid-5-yl | | | | |
| 187 | Tetrahydropyran-4-yl | 2-phenyl-5- | H | H, H | H | 480 |
| | | methyl- | | | | |
| | | oxazol-4-yl | | | | |
| 188 | Tetrahydropyran-4-yl | phenyl | H | H, H | H | 399 |
| | (1R isomer) | | | | | |
| 189 | Tetrahydropyran-4-yl | phenyl | H | H, H | H | 399 |
| | (1S isomer) | | | | | |
| 190 | 1-methyl-pyrazol-4-yl | 2,3-dihydro- | H | H, H | H | 453 |
| | | benzodioxan- | | | | |
| | | 5-yl | | | | |
| 191 | Tetrahydropyran-4-yl | 4-fluoro-3- | H | H, H | H | 447 |
| | | methoxy- | | | | |
| | | phenyl | | | | |
| 192 | 1-methyl-pyrazol-4-yl | 4-fluoro-3- | H | H, H | H | 443 |
| | | methoxy- | | | | |
| | | phenyl | | | | |
| 193 | 5-methyl-1,2,4- | 4-fluoro-3- | H | H, H | H | 445 |
| | oxadiazol-3-yl | methoxy- | | | | |
| | | phenyl | | | | |

The Examples shown in Table 3 were prepared from the appropriately substituted tert-butyl 2-(1H-indol-3-yl)-1-(4-aryl-1H-imidazol-2-yl)-1-ethylcarbamate derivative and a substituted heterocyclic or heteroaryl ketone according to the methods described in Examples 1-21.

TABLE 3

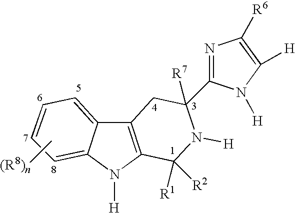

| | | | | | | LC-MS |
|---|---|---|---|---|---|---|
| | | | | | | m/z |
| Ex. No. | R¹ | R² | R⁶ | R⁷ | R⁸ | (M + H)+ |
| 194 | 3-methyl-1,2,4- | CH₃ | 4-fluoro- | H | H | 429.1 |
| | oxadiazol-5-yl | | phenyl | | | |
| 195 | 2-methyl-oxazol-4-yl | CH₃ | 4-fluoro- | H | H | 428.3 |
| | | | phenyl | | | |
| 196 | 2,5-dimethyl-oxazol-4-yl | CH₃ | 4-fluoro- | H | H | 442.4 |
| | | | phenyl | | | |
| 197 | 2,4-dimethyl-oxazol-5-yl | CH₃ | 4-fluoro- | H | H | 442.0 |
| | | | phenyl | | | |
| 198 | 5-methyl-1,2,4- | CH₃ | phenyl | H | H | 411.2 |
| | oxadiazol-3-yl | | | | | |
| 199 | 1-methyl-pyrazol-3-yl | CH₃ | 4-fluoro- | H | H | 427.1 |
| | | | phenyl | | | |
| 200 | 5-methyl-1,2,4- | CH₃ | 4-fluoro- | H | H | 429.2 |
| | oxadiazol-3-yl | | phenyl | | | |
| 201 | 5-methyl-1,3,4- | CH₃ | 4-fluoro- | H | H | 429.1 |
| | oxadiazol-2-yl | | phenyl | | | |
| 202 | 5-methyl-1,2,4- | CH₃ | phenyl | CH₃ | 7-F | 443.3 |
| | oxadiazol-3-yl | | | | | |
| 203 | 5-methyl-1,2,4- | 3-(methoxy- | 4-fluoro- | H | H | 515.4 |
| | oxadiazol-3-yl | carbonyl)- | phenyl | | | |
| | (isomer A) | 1-propyl | | | | |
| 204 | 5-methyl-1,2,4- | 3-(methoxy- | 4-fluoro- | H | H | 515.4 |
| | oxadiazol-3-yl | carbonyl)-1- | phenyl | | | |
| | (isomer B) | propyl | | | | |
| 205 | 5-methyl-1,2,4- | 3-carboxy- | 4-fluoro- | H | H | 501.4 |
| | oxadiazol-3-yl | 1-propyl | phenyl | | | |
| | (isomer A) | | | | | |
| 206 | 5-methyl-1,2,4- | 3-carboxy- | 4-fluoro- | H | H | 501.4 |
| | oxadiazol-3-yl | 1-propyl | phenyl | | | |
| | (isomer B) | | | | | |
| 207 | 5-methyl-1,2,4- | n-butyl | 4-fluoro- | H | H | 471.31 |
| | oxadiazol-3-yl | | phenyl | | | |
| | (isomer A) | | | | | |
| 208 | 5-methyl-1,2,4- | n-butyl | 4-fluoro- | H | H | 471.29 |
| | oxadiazol-3-yl | | phenyl | | | |
| | (isomer B) | | | | | |
| 209 | 5-methyl-1,2,4- | n-butyl | phenyl | H | H | 453.24 |
| | oxadiazol-3-yl | | | | | |
| | (isomer A) | | | | | |
| 210 | 5-methyl-1,2,4- | n-butyl | phenyl | H | H | 453.24 |
| | oxadiazol-3-yl | | | | | |
| | (isomer B) | | | | | |
| 211 | 5-methyl-1,2,4- | n-propyl | 4-fluoro- | H | H | 457.28 |
| | oxadiazol-3-yl | | phenyl | | | |
| | (isomer A) | | | | | |
| 212 | 5-methyl-1;2,4- | n-propyl | 4-fluoro- | H | H | 457.29 |
| | oxadiazol-3-yl | | phenyl | | | |
| | (isomer B) | | | | | |

The Examples shown in Table 4 were prepared from the appropriately substituted tert-butyl 2-(1H-indol-3-yl)-1-(4-aryl-1H-imidazol-2-yl)-1-ethylcarbamate derivative and a substituted heterocyclic or heteroaryl ketone according to the methods described in Examples 1-21.

TABLE 4

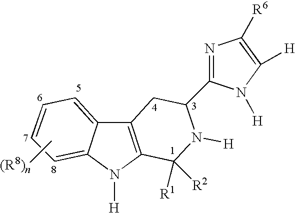

| | | | | | LC-MS |
|---|---|---|---|---|---|
| | | | | | m/z |
| Ex. No. | R¹ | R² | R⁶ | R⁸ | (M + H)+ |
| 213 | 3-methyl-1,2,4- | 3-methyl-1,2,4- | 4-fluoro- | H | 497.3 |
| | oxadiazol-5-yl | oxadiazol-5-yl | phenyl | | |
| 214 | 1-methyl-pyrazol-4-yl | 1-methyl-pyrazol-4-yl | 4-fluoro- | H | 493.3 |
| | | | phenyl | | |
| 215 | 1-methyl-pyrazol-4-yl | tetrahydropyran-4-yl | 4-fluoro- | H | 497.0 |
| | | | phenyl | | |
| 216 | 1-methyl-pyrazol-4-yl | ethoxycarbonyl | 4-fluoro- | H | 485.3 |
| | | | phenyl | | |
| 217 | 3-methyl-1,2,4- | 1-methyl-pyrazol-4-yl | phenyl | H | 477.3 |
| | oxadiazol-5-yl | | | | |
| 218 | 2-methyl-1,3,4- | 1-methyl-pyrazol-4-yl | 4-fluoro- | H | 495.3 |
| | oxadiazol-5-yl | | phenyl | | |
| 219 | 3-methyl-1,2,4- | tetrahydropyran-4-yl | 4-fluoro- | H | 499.4 |
| | oxadiazol-5-yl | | phenyl | | |
| 220 | 1-methyl-pyrazol-4- | pyrazin-2-yl | 4-fluoro- | H | 491.0 |
| | yl (Isomer A) | | phenyl | | |
| 221 | 1-methyl-pyrazol-4- | pyrazin-2-yl | 4-fluoro- | H | 491.0 |
| | yl (Isomer B) | | phenyl | | |
| 222 | 1-methyl-pyrazol-4-yl | 5-methyl-1,2,4- | 4-fluoro- | H | 511.0 |
| | | thiadiazol-3-yl | phenyl | | |
| 223 | 2-methyl-tetrazol-5-yl | tetrahydropyran-4-yl | 4-fluoro- | H | 499.3 |
| | | | phenyl | | |
| 224 | 1-methyl-pyrazol-4-yl | isopropoxycarbonyl | 4-fluoro- | H | 499.1 |
| | | | phenyl | | |
| 225 | 1-methyl-pyrazol-4-yl | pyrimidin-4-yl | 4-fluoro- | H | 491.2 |
| | | | phenyl | | |
| 226 | 1-methyl-pyrazol-4-yl | 2-methyl-tetrazol-5-yl | 4-fluoro- | H | 495.2 |
| | (Isomer A) | | phenyl | | |
| 227 | 1-methyl-pyrazol-4-yl | 2-methyl-tetrazol-5-yl | 4-fluoro- | H | 495.2 |
| | (Isomer B) | | phenyl | | |
| 228 | 2-methyl-tetrazol-5-yl | ethoxycarbonyl | 4-fluoro- | H | 487.2 |
| | (Isomer A) | | phenyl | | |
| 229 | 2-methyl-tetrazol-5-yl | ethoxycarbonyl | 4-fluoro- | H | 487.2 |
| | (Isomer B) | | phenyl | | |
| 230 | 1-methyl-pyrazol-4-yl | 2-hydroxy-1,3,4- | 4-fluoro- | H | 497.0 |
| | | oxadiazol-5-yl | phenyl | | |
| 231 | 1-methyl-pyrazol-4-yl | 5-methyl-1,2,4- | 4-fluoro- | 5-CH₃ | 509.20 |
| | | oxadiazol-3-yl | phenyl | | |
| 232 | 1-methyl-pyrazol-4- | 2-methyl-1,3,4- | 4-fluoro- | H | 495.4 |
| | yl (3S-isomer) | oxadiazol-5-yl | phenyl | | |
| 233 | 1-methyl-pyrazol-4-yl | ethoxycarbonyl- | 4-fluoro- | H | 499.3 |
| | | methyl | phenyl | | |
| 234 | 5-(1-hydroxy-1- | ethoxycarbonyl | 4-fluoro- | H | 517.4 |
| | methyl-ethyl)-1,2,4- | | phenyl | | |
| | oxadiazol-3-yl | | | | |
| 235 | 1-methyl-pyrazol-4-yl | carboxy-methyl | 4-fluoro- | H | 471.1 |
| | | | phenyl | | |
| 236 | 1-methyl-pyrazol-4-yl | pyrazin-2-yl | 4-fluoro- | 5-CH₃ | 505.1 |
| | | | phenyl | | |
| 237 | 1-methyl-pyrazol-4-yl | 6-ethoxycarbonyl- | 4-fluoro- | H | 562.2 |
| | | pyridin-2-yl | phenyl | | |
| 238 | 1-methyl-pyrazol-4-yl | 6-carboxy-pyridin- | 4-fluoro- | H | 534.2 |
| | | 2-yl | phenyl | | |

### Example 239

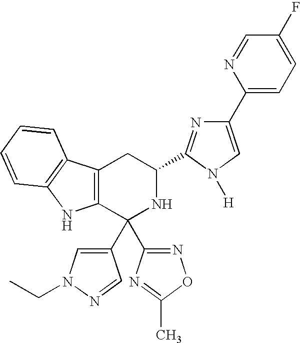

### (3R)-[4-(4-Fluorophenyl)-1H-imidazol-2-yl]-1-(5-methyl-1,2,4-oxadiazol-3-yl)-1-(1-ethyl-1H-pyrazol-4-yl)-2,3,4,9-tetrahydro-1H-β-carboline

### Step A: 2-Chloroacetyl-5-fluoropyridine

2-Bromo-5-fluoropyridine (50.0 g, 284 mmol) in 200 mL of THF was added drop-wise over 25 min to isopropylmagnesium chloride (2 M in THF, 284 mL, 568 mmol) at RT and the mixture was stirred for 2 h at RT. A solution of 2-chloro-N-methoxy-N-methylacetamide (119 g, 695 mmol) in 150 mL of THF was added dropwise over 30 min, to the reaction mixture at RT. The mixture was stirred at RT overnight. The mixture was poured into 2000 g of ice with 500 mL of 2 N HCl. The mixture was extracted into ether, washed with brine, dried over anhydrous sodium sulfate and concentrated to a residue, which was dissolved in 1 L of warm hexane and treated with several grams of silica gel to remove colored impurities. The mixture was then filtered. The filtrate was concentrated and chilled at ice temperature for 0.5 h. The solid was isolated by filtration to give 2-chloroacetyl-5-fluoropyridine. ¹H NMR (500 MHz, CDCl₃): 8.53 (d, 1H), 8.19 (dd, 1H), 7.60 (td, 1H), 5.09 (s, 2H).

### Step B: tert-Butyl 2-(1H-indol-3-yl)-1-(4-(5-fluoro-pyridin-2-yl)-1H-imidazol-2-yl)-1-ethylcarbamate

2-Chloroacetyl-5-fluoropyridine was converted into tert-butyl 2-(1H-indol-3-yl)-1-(4-(5-fluoro-pyridin-2-yl)-1H-imidazol-2-yl)-1-ethylcarbamate using procedures described in Gordon, T. et al., Bioorg. Med. Chem. Lett. 1993, 3, 915; Gordon, T. et al., Tetrahedron Lett. 1993, 34, 1901; and Poitout, L. et al., J. Med. Chem. 2001, 44, 2990. LC-MS: m/e 422.4 (M+H)⁺ (2.49 min).

### Step C: 2-(1H-Indol-3-yl)-1-(4-(5-fluoro-pyridin-2-yl)-1H-imidazol-2-yl)-ethylamine

tert-Butyl 2-(1-indol-3-yl)-1-(4-(5-fluoro-pyridin-2-yl)-1H-imidazol-2-yl)-1-ethylcarbamate (100 g, 237 mmol) was added to CH₃CN and stirred for 5 min. Additional CH₃CN was added gradually until total volume was 1.6 L. p-Toluenesulfonic acid monohydrate (149 g, 783 mmol) was added. The mixture was heated to 60° C. for 1 hr, then cooled to RT. The solid was separated by filtration, washed with CH₃CN, and air-dried to give 2-(1H-indol-3-yl)-1-(4-(5-fluoro-pyridin-2-yl)-1H-imidazol-2-yl)-ethylamine. LC-MS: m/e 322.4 (M+H)⁺ (1.92 min). ¹H NMR (500 MHz, CD₃OD): δ 8.54 (s, 1H), 8.05-7.97 (m, 2H), 7.89 (td, 1H), 7.69 (d, 4H), 7.43 (d, 1H), 7.31 (d, 1H), 7.18 (d, 4H), 7.10-7.03 (m, 2H), 6.95 (t, 1H), 5.03 (dd, 1H), 3.70-3.59 (m, 2H), 2.32 (s, 6H).

### Step D: 1-Ethyl-4-iodo-pyrazole

To a suspension of sodium hydride (2.68 g, 67.0 mmol) in DMF (100 mL) was added 4-iodo-pyrazole (10 g, 51.6 mmol) in portions while cooling in an ice-water bath. The mixture was heated to 60° C. for 30 min. The mixture was then cooled to 40° C. and ethyl iodide (8.33 mL, 103 mmol) was added. The reaction was heated to 40° C. for five h and then stirred overnight at RT. The reaction was quenched at 0° C. with dropwise addition of water. The mixture was extracted 4 times with EtOAc/hexanes. The combined organic layers were washed with water (3×) and brine, dried over anhydrous sodium sulfate, and evaporated under diminished pressure. Silica gel column chromatography eluted with 0% to 25% EtOAc/Hexanes afforded 1-ethyl-4-iodo-pyrazole. ¹H NMR (500 MHz, CDCl₃): δ 7.49 (s, 1H), 7.42 (s, 1H), 4.17 (q, 2H), 1.46 (t, 3H).

### Step E: N-Methoxy-N-methyl-5-methyl-1,2,4-oxadiazole-3-carboxamide

The title compound was prepared from 5-methyl-1,2,4-oxadiazole-3-carboxylic acid according to the procedures described for Intermediate 19, Step A.

### Step F: 1-Ethyl-pyrazol-4-yl 5-methyl-1,2,4-oxadiazol-3-yl ketone

The title compound was prepared from N-methoxy-N-methyl-5-methyl-1,2,4-oxadiazole-3-carboxamide according to the procedure described for Intermediate 22. To a solution of 1-ethyl-4-iodo-pyrazole (199 g, 807 mmol) in THF (2 L) at −10° C. was added isopropylmagnesium chloride (2M THF, 0.382 L, 765 mmol), dropwise over 20 min. The thick white mixture was stirred for 45 min at 0° C. The mixture was cooled to −70° C. and N-methoxy-N-methyl-5-methyl-1,2,4-oxadiazole-3-carboxamide in 130 ml THF was added dropwise over 10 min. The reaction was allowed to warm slowly to 0° C. over 3 h. The reaction was poured into 2.5 L of 1N HCl/ice and stirred for 30 min. The mixture was extracted two times with ethyl acetate. The combined organic layers were dried over anhydrous sodium sulfate and concentrated to a thick oil, which was diluted with about 1 L of hexane. The flask was placed on the rotary evaporator and slowly spun at about 30° C. for 30 min. The solids were broken up, filtered, washed with hexane, and air-dried to give 1-ethyl-pyrazol-4-yl 5-methyl-1,2,4-oxadiazol-3-yl ketone. ¹H NMR (500 MHz, CDCl₃): δ 8.41 (s, 1H), 8.29 (s, 1H), 4.23 (q, 2H), 2.69 (s, 3H), 1.53 (t, 3H).

### Step G: 3-[4-(5-Fluoro-pyridin-2-yl)-1H-imidazol-2-yl]-1-(5-methyl-1,2,4-oxadiazol-3-yl)-1-(1-ethyl-pyrazol-4-yl)-2,3,4,9-tetrahydro-1H-β-carboline

A mixture of 2-(1H-indol-3-yl)-1-(4-(5-fluoro-pyridin-2-yl)-1H-imidazol-2-yl)-ethylamine (95 g, 143 mmol), sodium acetate (11.71 g, 143 mmol), tetraethyl orthosilicate (29.7 g, 143 mmol) and 1-ethyl-pyrazol-4-yl 5-methyl-1,2,4-oxadiazol-3-yl ketone in DMSO (200 mL) was heated in an oil bath (75° C.) for 72 h. The reaction was cooled to RT and poured into 2N NaOH. The mixture was stirred for several min and then filtered. The filter cake was thoroughly washed with water and air dried to give a tan powder as a mixture of two diastereoisomers which was separated by SFC (analytical conditions: Chiral AD-H column, 4.6×250 mm, 40% (EtOH+0.2% isobutylamine)/CO₂, 2.1 mL/min, 100 bar, 40° C.; retention times were 5.53 min and 7.20 min for the two diastereoisomers, respectively). The fractions containing the fast eluting diastereoisomer were concentrated to give a solid. A portion of this material was recrystallized from acetonitrile/toluene followed by trituration with CH₂Cl₂ to give 3-[4-(5-fluoro-pyridin-2-yl)-1H-imidazol-2-yl]-1-(5-methyl-1,2,4-oxadiazol-3-yl)-1-(1-methyl-pyrazol-4-yl)-2,3,4,9-tetrahydro-1H-β-carboline. The rest of the material was recrystallized from CH₂Cl₂ to give additional 3-[4-(5-fluoro-pyridin-2-yl)-1H-imidazol-2-yl]-1-(5-methyl-1,2,4-oxadiazol-3-yl)-1-(1-ethyl-pyrazol-4-yl)-2,3,4,9-tetrahydro-1H-β-carboline. LC-MS: m/e 510.3 (M+H)⁺ (2.49 min). ¹H NMR (500 MHz, CD₃OD): δ 8.38 (s, 1H), 7.92-7.85 (m, 1H), 7.67 (s, 1H), 7.59 (td, 2H), 7.52-7.45 (m, 2H), 7.34 (d, 1H), 7.11 (t, 1H), 7.01 (t, 1H), 4.47 (dd, 1H), 4.12 (q, 2H), 3.21 (dd, 1H), 3.13 (dd, 1H), 2.59 (s, 3H), 1.40 (t, 3H).

From a separate reaction, the other diastereoisomer was also isolated. LC-MS: ink 510.4 (M+H)⁺ (2.57 min). ¹H NMR (500 MHz, CD₃OD): δ 8.36 (d, 1H), 7.85 (d, 1H), 7.59-7.50 (m, 2H), 7.50-7.41 (m, 3H), 7.36 (d, 1H), 7.11 (t, 1H), 7.02 (t, 1H), 4.39 (dd, 1H), 4.06 (q, 2H), 3.23 (dd, 1H), 3.12 (dd, 1H), 2.56 (s, 3H), 1.35 (t, 3H).

### Example 240

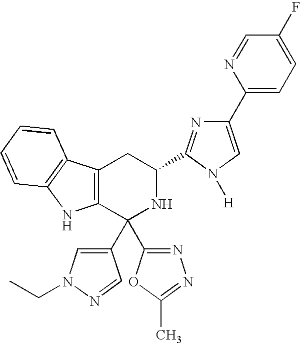

### (3R)-[4-(4-Fluorophenyl)-1H-imidazol-2-yl]-1-(5-methyl-1,3,4-oxadiazol-3-yl)-1-(1-ethyl-1H-pyrazol-4-yl)-2,3,4,9-tetrahydro-1H-β-carboline

### Step A: N-Methoxy-N-methyl-5-methyl-1,3,4-oxadiazole-2-carboxamide

The title compound was prepared according to the procedure described for the preparation of Intermediate 19, Step A. A mixture of 1,3,4-oxadiazole-2-carboxylic acid, potassium salt (29.3 g, 176 mmol) in CH₂Cl₂ (500 ml) and DMF (1.365 ml, 17.63 mmol) was cooled to 0° C. and oxalyl chloride (18.52 ml, 212 mmol) was added dropwise over 20 min. The reaction mixture was warmed to RT and stirred for 1 h. This acid chloride solution was added to a cooled solution of N,O-dimethylhydroxylamine HCl (27.5 g, 282 mmol) and K₂CO₃ (110 g, 793 mmol) in water (300 mL). The mixture was stirred at RT for 3 h. The organic layer was washed with brine, dried, filtered and concentrated to give the crude N-methoxy-N-methyl-5-methyl-1,3,4-oxadiazole-2-carboxamide which was purified by MPLC (10% EtOAc in hexane to 100% EtOAc) to afford N-methoxy-N-methyl-5-methyl-1,3,4-oxadiazole-2-carboxamide.

¹H NMR (500 MHz, CDCl₃): δ 3.82 (s, 3H), 3.30 (s, 3H), 2.54 (s, 3H).

### Step B: 1-Ethyl-pyrazol-4-yl 5-methyl-1,3,4-oxadiazol-2-yl ketone

To a solution of 1-ethyl-4-iodo-pyrazole from Example 239, Step D (4.2 g, 18.92 mmol) in THF (50 mL) was added isopropylmagnesium chloride 2.0M in THF (10.40 mL, 20.81 mmol) at 0° C. The mixture was stirred at 0° C. for 1 h, cooled to −78° C., and N-methoxy-N-methyl-5-methyl-1,3,4-oxadiazole-2-carboxamide (2.266 g, 13.24 mmol) was added. The mixture was slowly warmed to RT in 4.5 h. The reaction was cooled to −78° C. and quenched by dropwise addition of saturated aqueous ammonium chloride and warmed to RT. The mixture was diluted with cold 1N HCl, extracted with EtOAc 4 times, and the combined organic layers were washed with brine and dried over anhydrous sodium sulfate. The residue was purified by MPLC on silica gel eluted with a gradient of 10% EtOAc in hexanes to 100% EtOAc to afford 1-ethyl-pyrazol-4-yl 5-methyl-1,3,4-oxadiazol-2-yl ketone. ¹H NMR (500 MHz, CDCl₃): δ 8.43 (s, 1H), 8.10 (s, 1H), 4.08 (q, 2H), 2.47 (s, 3H), 1.36 (t, 3H).

### Step C: 3-[4-(5-Fluoro-pyridin-2-yl)-1H-imidazol-2-yl]-1-(5-methyl-1,3,4-oxadiazol-2-yl)-1-(1-ethyl-pyrazol-4-yl)-2,3,4,9-tetrahydro-1H-β-carboline

2-(1H-Indol-3-yl)-1-(4-(5-fluoro-pyridin-2-yl)-1H-imidazol-2-yl)-ethylamine from Example 239, Step C (1.54 g, 2.313 mmol) was treated with tetraethoxysilane (1.295 ml, 5.78 mmol), 1-ethyl-pyrazol-4-yl 5-methyl-1,3,4-oxadiazol-2-yl ketone (0.620 g, 3.01 mmol) and pyridine (7 mL). The mixture was heated at 65° C. for 2.5 days. The mixture was treated with EtOAc and ice, followed by 5 N NaOH. The mixture was extracted with EtOAc, dried over anhydrous sodium sulfate and concentrated. The residue was purified by MPLC on silica gel eluted with a gradient of 20% acetone in CH₂Cl₂ to 100% acetone to give a mixture of two diastereoisomers. These diastereoisomers were subsequently separated on a Gilson HPLC using ChiralPak® AD column (analytical conditions: ChiralPak® AD 4.6×250 mm, 10μ, 30% IPA/heptane, 0.5 mL/min; retention times were 15.44 min and 23.87 min for the two diastereoisomers, respectively). The fractions containing the fast eluting diastereoisomer were combined to afford 3-[4-(5-fluoro-pyridin-2-yl)-1H-imidazol-2-yl]-1-(5-methyl-1,3,4-oxadiazol-2-yl)-1-(1-ethyl-pyrazol-4-yl)-2,3,4,9-tetrahydro-1H-β-carboline. LC-MS: m/e 510.3 (M+H)⁺ (1.01 min with 2 min gradient method). ¹H NMR (500 MHz, CD₃OD): δ 8.40 (s, 1H), 7.95-7.89 (m, 1H), 7.71 (s, 1H), 7.61 (td, 2H), 7.52 (d, 2H), 7.36 (d, 1H), 7.14 (t, 1H), 7.04 (t, 1H), 4.52 (dd, 1H), 4.15 (q, 2H), 3.28-3.14 (m, 2H), 2.54 (s, 3H), 1.42 (t, 3H).

From a separate reaction, the slow eluting diastereoisomer was also isolated. LC-MS: m/e 510.4 (M+H)⁺ (1.02 min with 2 min gradient method). ¹H NMR (500 MHz, CD₃OD): δ 8.35 (s, 1H), 7.85 (s, 1H), 7.59-7.46 (m, 5H), 7.36 (d, 1H), 7.13 (t, 1H), 7.03 (t, 1H), 4.38 (dd, 1H), 4.07 (q, 2H), 3.23 (dd, 1H), 3.12 (dd, 1H), 2.49 (s, 3H), 1.36 (q, 3H).

### Example 241

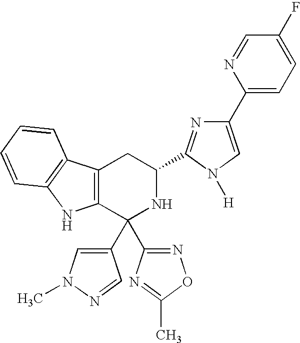

### 3-[4-(5-Fluoro-pyridin-2-yl)-1H-imidazol-2-yl]-1-(5-methyl-1,2,4-oxadiazol-3-yl)-1-(1-methyl-pyrazol-4-yl)-2,3,4,9-tetrahydro-1H-β-carboline

The bis-tosylate salt of 2-(1H-indol-3-yl)-1-(4-(5-fluoro-pyridin-2-yl)-1H-imidazol-2-yl)-ethylamine from Example 239, Step C (5.07 g, 7.62 mmol) was treated with sodium acetate (0.937 g, 11.42 mmol), tetraethoxysilane (2.56 ml, 11.42 mmol), 1-methyl-pyrazol-4-yl 5-methyl-1,2,4-oxadiazol-3-yl ketone (Intermediate 22) (1.756 g, 9.14 mmol) and DMSO (20 mL). The mixture was heated at 95° C. for 48 h. The mixture was cooled to RT. Water was added and the mixture extracted three times with ethyl acetate. The combined organic extracts were washed with water and dried over anhydrous sodium sulfate. The solvent was removed by rotoevaporation and the crude product purified by silica gel chromatography using MPLC (eluted with a gradient of EtOAc (100%) to 10% MeOH in EtOAc) to afford fractions enriched in the desired product. This material was further purified with preparative thin layer chromatography eluted with 12.5:1=CH₂Cl₂:(9:1 MeOH/NH₄OH) to afford 3-[4-(5-fluoro-pyridin-2-yl)-1H-imidazol-2-yl]-1-(5-methyl-1,2,4-oxadiazol-3-yl)-1-(1-methyl-pyrazol-4-yl)-2,3,4,9-tetrahydro-1H-β-carboline. Furthermore, mixed fractions of the desired product and its diastereoisomer from the MPLC chromatography was separated on Chiral OD SFC (40% IPA) to give the slower eluting desired diastereoisomer which was further purified by silica gel MPLC (eluted with CH₂Cl₂ gradient to acetone) to afford additional 3-[4-(5-fluoro-pyridin-2-yl)-1H-imidazol-2-yl]-1-(5-methyl-1,2,4-oxadiazol-3-yl)-1-(1-methyl-pyrazol-4-yl)-2,3,4,9-tetrahydro-1H-β-carboline.

LC-MS: m/e 496.3 (M+H)⁺ (1.00 min, 2 min method). ¹H NMR (500 MHz, MeOH-d₄): δ 8.40 (s, 1H), 7.93 (brs, 1H), 7.64-7.57 (m, 3H), 7.53-7.47 (m, 2H), 7.35 (d, 1H), 7.12 (t, 1H), 7.02 (t, 1H), 4.47 (dd, 1H), 3.85 (s, 3H), 3.22 (dd, 1H), 3.14 (dd, 1H), 2.60 (s, 3H).

The Examples shown in Table 5 were prepared from the appropriately substituted 2-(1H-indol-3-yl)-1-(4-(5-fluoro-pyridin-2-yl)-1H-imidazol-2-yl)-1-ethylamine derivative and a substituted heterocyclic or heteroaryl ketone according to the methods described in Examples 239-241.

TABLE 5

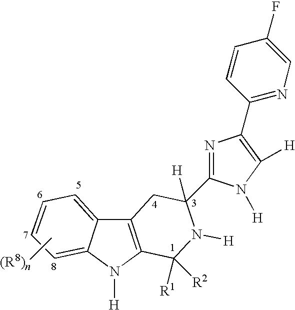

| | | | | LC-MS |
|---|---|---|---|---|
| | | | | m/z |
| Ex. No. | R¹ | R² | R⁸ | (M + H)+ |
| 242 | 1-methyl-pyrazol-4-yl | ethoxymethyl | H | 472.0 |
| 243 | 5-methyl-1,2,4-oxadiazol-3-yl | ethoxymethyl | H | 474.5 |
| 244 | 5-methyl-1,3,4-oxadiazol-2-yl | n-butyl | H | 472.3 |
| 245 | 1-methyl-pyrazol-4-yl | 5-methyl-1,3,4-oxadiazol-2-yl | H | 496.3 |
| 246 | 1-ethyl-pyrazol-4-yl | ethoxymethyl | H | 486.3 |
| 247 | 4,5-dihydro- 1-methyl-1H- | n-butyl | H | 500.0 |
| | pyridazin-6-on-3-yl | | | |
| 248 | 1-methyl-pyrazol-4-yl | 3-methyl-1,2,4-oxadiazol-5-yl | H | 496.1 |
| 249 | 5-methyl-1,3,4-oxadiazol-2- | tetrahydropyran-4-yl | 4-CN | 525.3 |
| | yl (Isomer A) | | | |
| 250 | 5-methyl-1,3,4-oxadiazol-2- | tetrahydropyran-4-yl | 4-CN | 525.3 |
| | yl (Isomer B) | | | |
| 251 | 1-methyl-pyrazol-4-yl | 2-pyridazinyl | H | 492.4 |
| 252 | 1-methyl-pyrazol-4-yl | 5-methyl-1,2,4-oxadiazol-3-yl | 5-F | 514.1 |
| | (Isomer A) | | | |
| 253 | 1-methyl-pyrazol-4-yl | 5-methyl-1,2,4-oxadiazol-3-yl | 5-F | 514.1 |
| | (Isomer A) | | | |
| 254 | 1-ethyl-pyrazol-4-yl | 2-methoxy-pyridin-5 -yl | H | 535.0 |

### Example 255

### Effects of a Combination of SSTR3 Antagonists and Dipeptidyl Peptidase-IV (DPP-4) Inhibitors on Oral Glucose Tolerance in Mice

Compounds of the present invention were combined with dipeptidyl peptidase-IV (DPP-4) inhibitors in oral glucose tolerance test (oGTT) described above. Male C57BL/6N mice (7-12 weeks of age) were housed 10 per cage and given access to normal rodent chow and water ad libitum. Mice were randomly assigned to treatment groups and fasted 4 to 6 h. Baseline blood glucose concentrations were determined by glucometer from tail nick blood. Animals were then treated orally with vehicle (0.25% methylcellulose) or test compound alone or in combination with a dipeptidyl peptidase-IV inhibitor. Blood glucose concentration was measured at a set time point after treatment (t=0 min) and mice were then challenged with dextrose intraperitoneally (2-3 g/kg) or orally (3-5 g/kg). One group of vehicle-treated mice was challenged with saline as a negative control. Blood glucose levels were determined from tail bleeds taken at 20, 40, 60 min after dextrose challenge. The blood glucose excursion profile from t=0 to t=90 min was used to integrate an area under the curve (AUC) for each treatment. Percent inhibition values for each treatment were generated from the AUC data normalized to the saline-challenged controls. Suboptimal doses of Examples 20 and 21 in the range of 0.001 to 0.1 mg/kg po were found to be more active in combination with low doses of a DPP-4 inhibitor, such as sitagliptin and des-fluoro-sitagliptin, that is, (2R)-1-(2,5-difluorophenyl)-4-oxo-4-[3-(trifluoromethyl)-5,6-dihydro[1,2,4]triazolo[4,3-a]pyrazin-7(8H)-2-amine, than they were alone.

### Examples of Pharmaceutical Formulations

As a specific embodiment of an oral composition of a compound of the present invention, 50 mg of the compound of any of the Examples is formulated with sufficient finely divided lactose to provide a total amount of 580 to 590 mg to fill a size 0 hard gelatin capsule.

As a second specific embodiment of an oral composition of a compound of the present invention, 100 mg of the compound of any of the Examples, microcrystalline cellulose (124 mg), croscarmellose sodium (8 mg), and anhydrous unmilled dibasic calcium phosphate (124 mg) are thoroughly mixed in a blender; magnesium stearate (4 mg) and sodium stearyl fumarate (12 mg) are then added to the blender, mixed, and the mix transferred to a rotary tablet press for direct compression. The resulting tablets are optionally film-coated with Opadry® II for taste masking.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable

1. A compound of structural formula I: 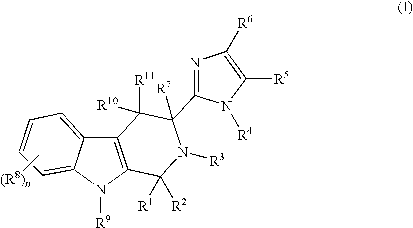
or a pharmaceutically acceptable salt thereof, wherein:
n is an integer from 1 to 4;
R¹ is selected from the group consisting of:
(1) —C(O)OR^{e},
(2) —C(O)NR^{c}R^{d},
(3) cycloheteroalkyl,
(4) cycloheteroalkyl-C₁₋₁₀ alkyl-,
(5) heteroaryl, and
(6) heteroaryl-C₁₋₁₀ alkyl-;
wherein alkyl and cycloheteroalkyl are optionally substituted with one to three substituents independently selected from R^{a}; and heteroaryl is optionally substituted with one to three substituents independently selected from R^{b};
with the proviso that heteroaryl is not pyridinyl, pyrrolyl, thienyl, 1,3-benzodioxolyl, or furanyl;
R² is selected from the group consisting of
(1) hydrogen,
(2) C₁₋₁₀ alkyl,
(3) C₂₋₁₀ alkenyl,
(4) C₂₋₁₀ alkynyl,
(5) C₃₋₁₀ cycloalkyl,
(6) C₃₋₁₀ cycloalkyl-C₁₋₁₀ alkyl-,
(7) C₁₋₆ alkyl-X—C₁₋₆ alkyl-,
(8) aryl-C₁₋₄ alkyl-X—C₁₋₄ alkyl-,
(9) heteroaryl-C₁₋₄ alkyl-X—C₁₋₄ alkyl-,
(10) C₃₋₁₀ cycloalkyl-X—C₁₋₆ alkyl-,
(11) aryl,
(12) cycloheteroalkyl, and
(13) heteroaryl;
wherein X is selected from the group consisting of O, S, S(O), S(O)₂, and NR⁴ and wherein alkyl, alkenyl, alkynyl, cycloalkyl, and cycloheteroalkyl are optionally substituted with one to three substituents independently selected from R^{a}; and aryl and heteroaryl are optionally substituted with one to three substituents independently selected from R^{b};
R³ is selected from the group consisting of
(1) hydrogen,
(2), C₁₋₁₀ alkyl,
(3) C₃₋₁₀ cycloalkyl,
(4) cycloheteroalkyl,
(5) cycloheteroalkyl-C₁₋₆ alkyl-, and
(6) heteroaryl-C₁₋₆ alkyl-;
wherein alkyl, cycloalkyl, and cycloheteroalkyl are optionally substituted with one to three substituents independently selected from R^{a}; and heteroaryl is optionally substituted with one to three substituents independently selected from R^{b};
R⁴ is hydrogen or C₁₋₈ alkyl, optionally substituted with one to five fluorines;
R⁵ and R⁶ are each independently selected from the group consisting of
(1) hydrogen,
(2) C₁₋₁₀ alkyl,
(3) C₂₋₁₀ alkenyl,
(4) C₂₋₁₀ alkynyl,
(5) C₃₋₁₀ cycloalkyl,
(6) cycloheteroalkyl,
(7) aryl, and
(8) heteroaryl;
wherein alkyl, cycloalkyl, and cycloheteroalkyl are optionally substituted with one to three substituents independently selected from R^{a}, and aryl and heteroaryl are optionally substituted with one to three substituents independently selected from R^{b};
R⁷ is selected from the group consisting of
(1) hydrogen,
(2) C₁₋₁₀ alkyl, optionally substituted with one to five fluorines,
(3) C₂₋₁₀ alkenyl,
(4) C₃₋₁₀ cycloalkyl, and
(5) C₁₋₄ alkyl-O—C₁₋₄ alkyl-;
each R⁸ is independently selected from the group consisting of
(1) hydrogen,
(2) —OR^{e},
(3) —NR^{c}S(O)ₘR^{e},
(4) halogen,
(5) —S(O)ₘR^{e},
(6) —S(O)ₘNR^{c}R^{d},
(7) —NR^{c}R^{d},
(8) —C(O)R^{e},
(9) —OC(O)R^{e},
(10) —CO₂R^{e},
(11) —CN,
(12) —C(O)NR^{c}R^{d},
(13) —NR^{c}C(O)R^{e},
(14) —NR^{c}C(O)OR^{e},
(15) —NR^{c}C(O)NR^{c}R^{d},
(16) —OCF₃,
(17) —OCHF₂,
(18) cycloheteroalkyl,
(19) C₁₋₁₀ alkyl, optionally substituted with one to five fluorines,
(20) C₃₋₆ cycloalkyl,
(21) aryl, and
(22) heteroaryl;
wherein aryl and heteroaryl are optionally substituted with one to three substituents independently selected from R^{b};
R⁹ is selected from the group consisting of
(1) hydrogen,
(2) C₁₋₁₀ alkyl,
(3) C₂₋₁₀ alkenyl, and
(4) C₃₋₁₀ cycloalkyl;
wherein alkyl, alkenyl, and cycloalkyl are optionally substituted with one to three substituents independently selected from R^{a};
R¹⁰ and R¹¹ are each independently hydrogen or C₁₋₄ alkyl, optionally substituted with one to five fluorines;
each R^{a} is independently selected from the group consisting of:
(1) —OR^{e},
(2) —NR^{c}S(O)ₘR^{e},
(3) halogen,
(4) —S(O)ₘR^{e},
(5) —S(O)ₘNR^{c}R^{d},
(6) —NR^{c}R^{d},
(7) —C(O)R^{e},
(8) —OC(O)R^{e},
(9) oxo,
(10) —CO₂R^{e},
(11) —CN,
(12) —C(O)NR^{c}R^{d},
(13) —NR^{c}C(O)R^{e},
(14) —NR^{c}C(O)OR^{e},
(15) —NR^{c}C(O)NR^{c}R^{d},
(16) —CF₃,
(17) —OCF₃,
(18) —OCHF₂,
(19) cycloheteroalkyl;
(20) C₃₋₆ cycloalkyl-C₁₋₆ alkyl; and
(21) C₁₋₆ alkyl-X—C₁₋₆ alkyl-;
wherein X is selected from the group consisting of O, S, S(O), S(O)₂, and NR⁴;
each R^{b} is independently selected from the group consisting of:
(1) R^{a},
(2) C₁₋₁₀ alkyl, and
(3) C₃₋₆ cycloalkyl;
wherein alkyl and cycloalkyl are optionally substituted with one to three hydroxyls and one to six fluorines;
R^{c} and R^{d} are each independently selected from the group consisting of
(1) hydrogen,
(2) C₁₋₁₀ alkyl,
(3) C₂₋₁₀ alkenyl,
(4) C₃₋₆ cycloalkyl,
(5) C₃₋₆ cycloalkyl-C₁₋₁₀ alkyl-,
(6) cycloheteroalkyl,
(7) cycloheteroalkyl-C₁₋₁₀ alkyl-,
(8) aryl,
(9) heteroaryl,
(10) aryl-C₁₋₁₀ alkyl-, and
(11) heteroaryl-C₁₋₁₀ alkyl-; or
R^{c} and R^{d} together with the atom(s) to which they are attached form a heterocyclic ring of 4 to 7 members containing 0-2 additional heteroatoms independently selected from oxygen, sulfur and N—R^{g}; and, when R^{c} and R^{d} are other than hydrogen, each R^{c} and R^{d} is optionally substituted with one to three substituents independently selected from R^{h};
each R^{e} is independently selected from the group consisting of:
(1) hydrogen,
(2) C₁₋₁₀ alkyl,
(3) C₂₋₁₀ alkenyl,
(4) C₃₋₆ cycloalkyl,
(5) C₃₋₆ cycloalkyl-C₁₋₁₀ alkyl-,
(6) cycloheteroalkyl,
(7) cycloheteroalkyl-C₁₋₁₀ alkyl-,
(8) aryl,
(9) heteroaryl,
(10) aryl-C₁₋₁₀ alkyl-, and
(11) heteroaryl-C₁₋₁₀ alkyl-;
wherein, when R^{e} is not hydrogen, each R^{e} is optionally substituted with one to three substituents selected from R^{h};
each R^{g} is independently —C(O)R^{e} or C₁₋₁₀ alkyl, optionally substituted with one to five fluorines;
each R^{h} is independently selected from the group consisting of:
(1) halogen,
(2) C₁₋₁₀ alkyl,
(3) —O—C₁₋₄ alkyl,
(4) —S(O)ₘ—C₁₋₄ alkyl,
(5) —CN,
(6) —CF₃,
(7) —OCHF₂, and
(8) —OCF₃; and
each m is independently 0, 1 or 2.

2. The compound of claim 1 wherein R³, R⁴, R⁵, R⁹, R¹⁰, and R¹¹ are each hydrogen, or a pharmaceutically acceptable salt thereof.

3. The compound of claim 2 wherein R⁷ is hydrogen or methyl, or a pharmaceutically acceptable salt thereof.

4. The compound of claim 1 wherein R⁴ and R⁵ are hydrogen, and R⁶ is phenyl or heteroaryl each of which is optionally substituted with one to three substituents independently selected from R^{b}, or a pharmaceutically acceptable salt thereof.

5. The compound of claim 4 wherein heteroaryl is pyridinyl optionally substituted with one to two substituents independently selected from R^{b}, or a pharmaceutically acceptable salt thereof.

6. The compound of claim 4 wherein R⁶ is phenyl or pyridin-2-yl optionally substituted with one to two substituents independently selected from the group consisting of halogen, methyl, and methoxy, or a pharmaceutically acceptable salt thereof.

7. The compound of claim 6 wherein R⁶ is phenyl, 4-fluorophenyl, pyridin-2-yl, or 5-fluoro-pyridin-2-yl, or a pharmaceutically acceptable salt thereof.

8. The compound of claim 1 wherein n is 1, or a pharmaceutically acceptable salt thereof.

9. The compound of claim 8 wherein R⁸ is hydrogen, halogen, or cyano, or a pharmaceutically acceptable salt thereof.

10. The compound of claim 1 wherein R² is selected from the group consisting of:
(1) hydrogen,
(2) heteroaryl, optionally substituted with one to three substituents independently selected from Rb,
(3) C₁₋₃ alkyl-O—C₁₋₃ alkyl-, and
(4) C₁₋₆ alkyl, wherein alkyl is optionally substituted with one to two substituents independently selected from R^{a}, or a pharmaceutically acceptable salt thereof.

11. The compound of claim 1 wherein R¹ is cycloheteroalkyl or heteroaryl wherein cycloheteroalkyl is optionally substituted with one to three substituents independently selected from R^{a}, and heteroaryl is optionally substituted with one to three substituents independently selected from R^{b}, or a pharmaceutically acceptable salt thereof.

12. The compound of claim 11 wherein R¹ is heteroaryl optionally substituted with one to two substituents independently selected from R^{b}, or a pharmaceutically acceptable salt thereof.

13. The compound of claim 12 wherein R¹ is heteroaryl selected from the group consisting of 1,2,4-oxadiazol-3-yl, 1,3,4-oxadiazol-2-yl, 1,2,4-thiadiazol-3-yl, pyrazol-3-yl, pyrazol-4-yl, 1,2,3-triazol-4-yl, 1,2,4-triazol-3-yl, 1,3-thiazol-4-yl, 1,3-thiazol-5-yl, and 1,3-oxazol-4-yl, each of which is optionally substituted with C₁₋₄ alkyl wherein alkyl is optionally substituted with one to three fluorines, or a pharmaceutically acceptable salt thereof.

14. The compound of claim 1 wherein R¹ is heteroaryl optionally substituted with one to three substituents independently selected from R^{b}; and R² is selected from the group consisting of:
(1) hydrogen,
(2) heteroaryl, optionally substituted with one to three substituents independently selected from R^{b},
(3) C₁₋₃ alkyl-O—C₁₋₃ alkyl-, and
(4) C₁₋₆ alkyl, wherein alkyl is optionally substituted with one to two substituents independently selected from R^{a}, or a pharmaceutically acceptable salt thereof.

15. The compound of claim 14 wherein R¹ or R² is hydrogen, or a pharmaceutically acceptable salt thereof.

16. The compound of claim 15 wherein R² is heteroaryl optionally substituted with one to three substituents independently selected from R^{b}, or a pharmaceutically acceptable salt thereof.

17. The compound of claim 1 of structural formula II having the indicated R stereochemical configuration at the stereogenic carbon atom marked with an *: 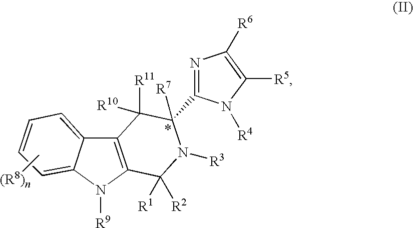
or a pharmaceutically acceptable salt thereof.

18. The compound of claim 17 wherein R³, R⁴, R⁵, R⁹, R¹⁰, and R¹¹ are each hydrogen; R⁷ is hydrogen or methyl; and n is 1, or a pharmaceutically acceptable salt thereof.

19. The compound of claim 18 wherein R⁸ is hydrogen, halogen, or cyano, or a pharmaceutically acceptable salt thereof.

20. The compound of claim 17 wherein
R¹ is heteroaryl optionally substituted with one to three substituents independently selected from R^{b}, and
R² is selected from the group consisting of:
(1) hydrogen,
(2) heteroaryl, optionally substituted with one to three substituents independently selected from R^{b},
(3) C₁₋₃ alkyl-O—C₁₋₃ alkyl-, and
(4) C₁₋₆ alkyl, wherein alkyl is optionally substituted with one to two substituents independently selected from R^{a}, or a pharmaceutically acceptable salt thereof.

21. The compound of claim 20 wherein R¹ or R² is hydrogen, or a pharmaceutically acceptable salt thereof.

22. The compound of claim 20 wherein R² is heteroaryl optionally substituted with one to two substituents independently selected from R^{b}, or a pharmaceutically acceptable salt thereof.

23. The compound of claim 22 wherein R¹ and R² are each independently heteroaryl selected from the group consisting of 1,2,4-oxadiazol-3-yl, 1,3,4-oxadiazol-2-yl, 1,2,4-thiadiazol-3-yl, pyrazol-3-yl, pyrazol-4-yl, 1,2,3-triazol-4-yl, 1,2,4-triazol-3-yl, 1,3-thiazol-4-yl, 1,3-thiazol-5-yl, and 1,3-oxazol-4-yl, each of which is optionally substituted with C₁₋₄ alkyl wherein alkyl is optionally substituted with one to five fluorines, or a pharmaceutically acceptable salt thereof.

24. The compound of claim 1 selected from the group consisting of: 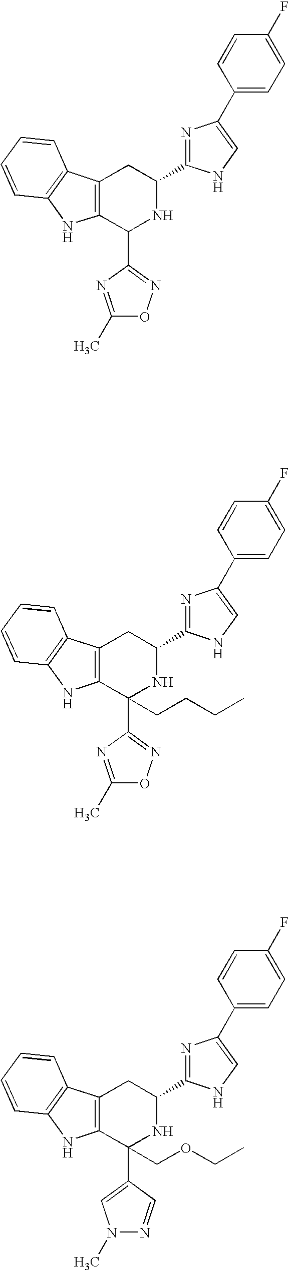 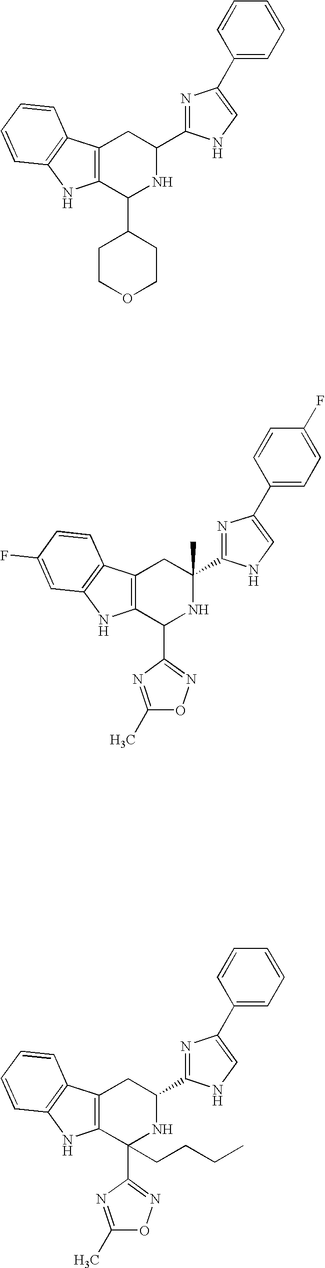 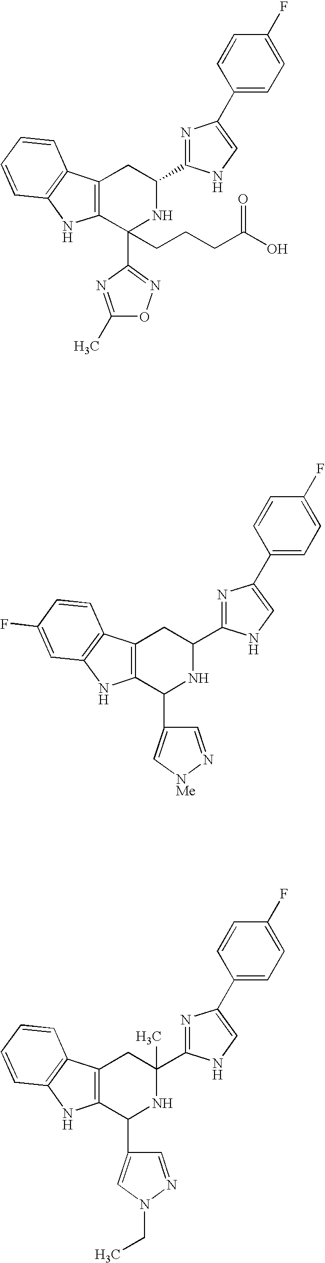 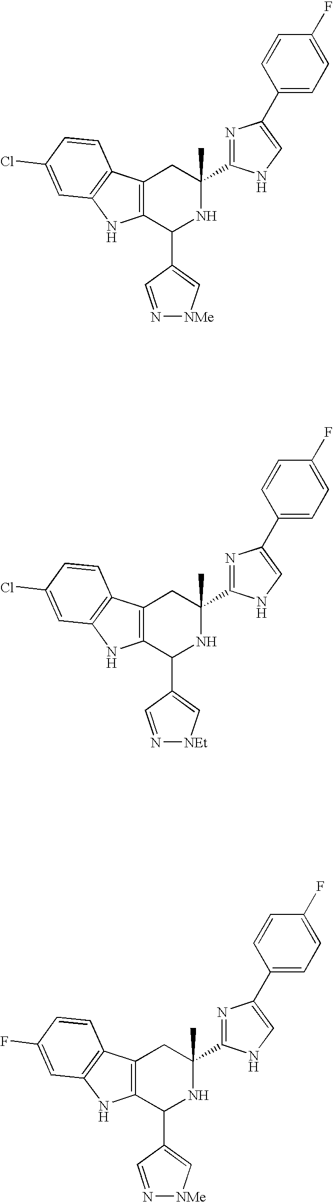 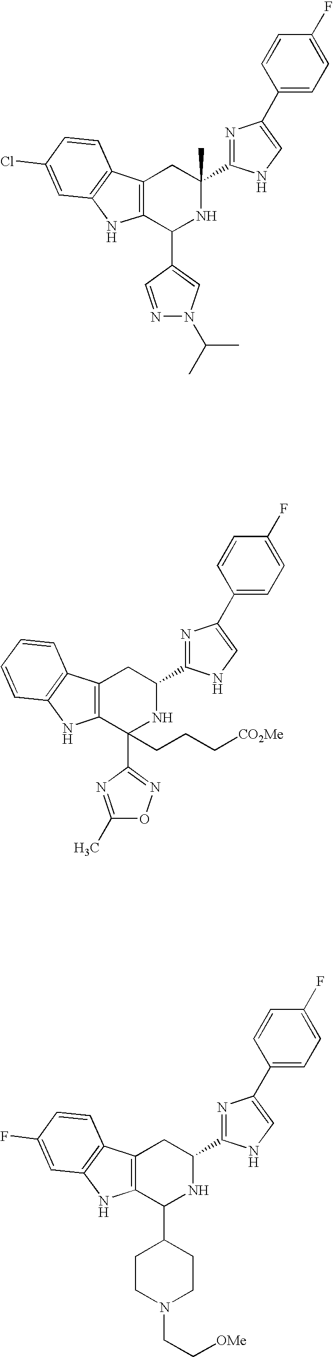 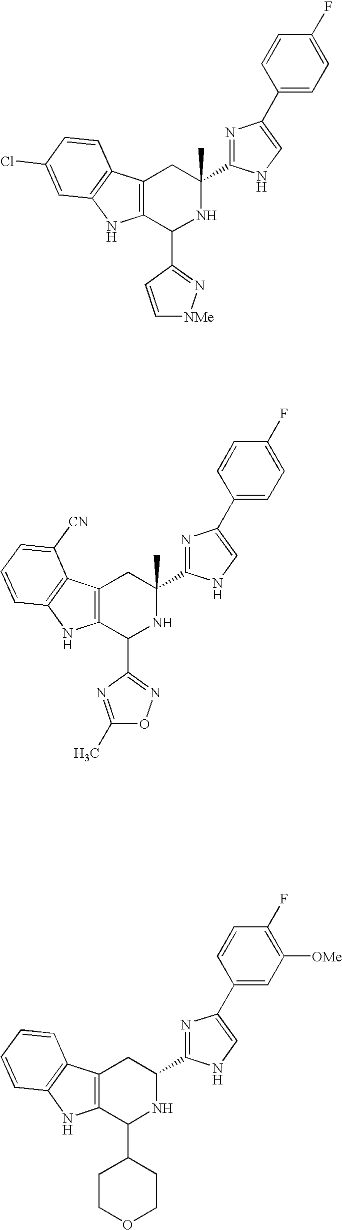 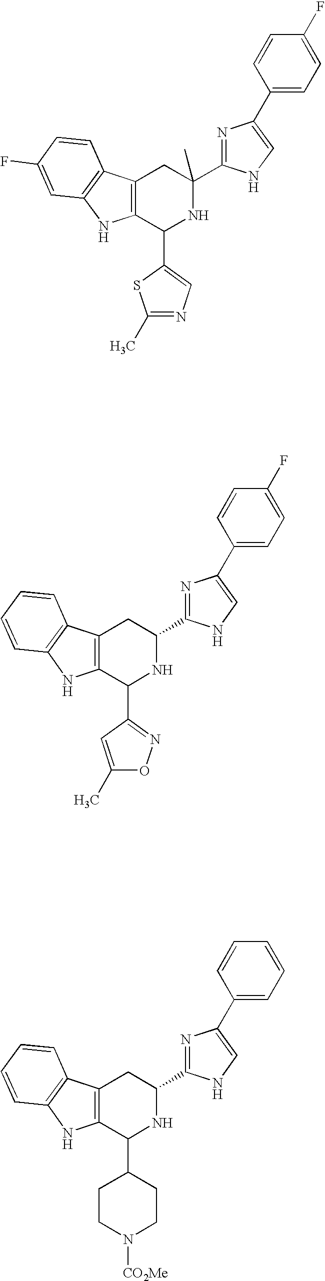 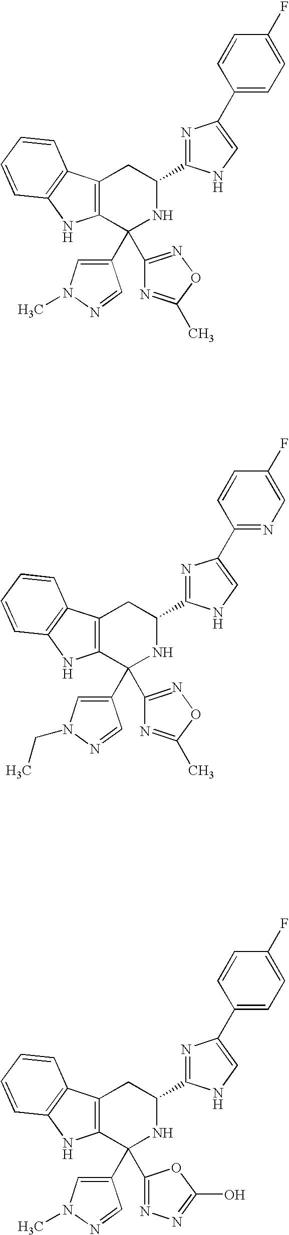 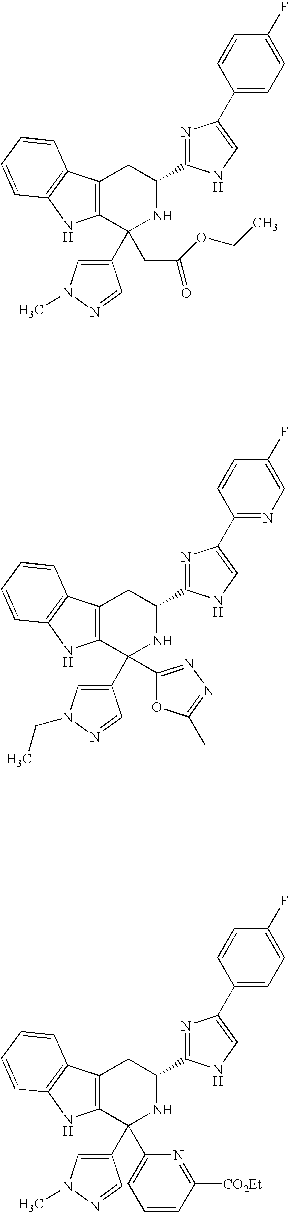 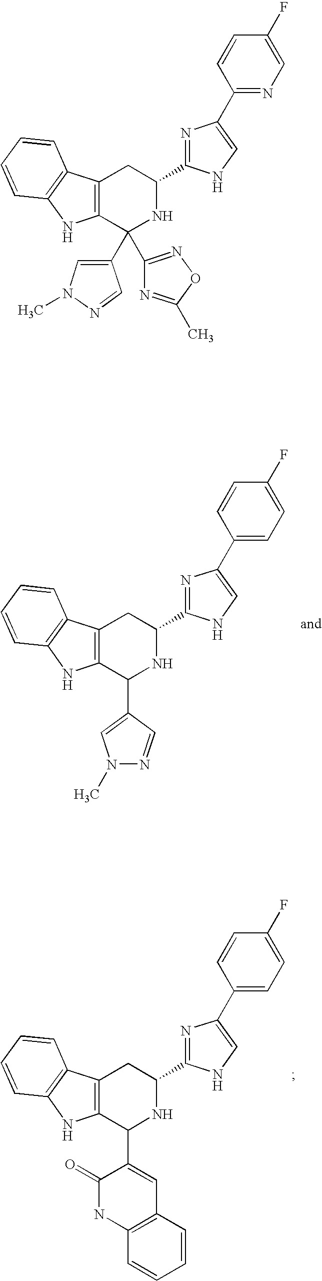
or a pharmaceutically acceptable salt thereof.

25. A pharmaceutical composition comprising a compound in accordance with claim 1, or a pharmaceutically acceptable salt thereof, in combination with a pharmaceutically acceptable carrier.

26-28. (canceled)

29. A method of treating a disorder, condition, or disease responsive to antagonism of the somatostatin subtype receptor 3 (SSTR3) in a subject in need thereof comprising administration of a therapeutically effective amount of a compound according to claim 1, or a pharmaceutically acceptable salt thereof.

30. The method according to claim 29 wherein the disorder, condition, or disease is selected from the group consisting of Type 2 diabetes, hyperglycemia, insulin resistance, obesity, a lipid disorders, Metabolic Syndrome, and hypertension.
